(19) United States
(12) Patent Application Publication
(10) Pub. No.: US 2026/0092252 A1
(43) Pub. Date: Apr. 2, 2026

(54) NETRIN-1 MIMETIC PEPTIDE AMPHIPHILES

(71) Applicant: Northwestern University, Evanston, IL (US)

(72) Inventors: Samuel Isaac Stupp, Evanston, IL (US); Cara Stephanie Smith, Evanston, IL (US); Zaida Alvarez Pinto, Evanston, IL (US)

(21) Appl. No.: 19/345,142

(22) Filed: Sep. 30, 2025

Related U.S. Application Data
(60) Provisional application No. 63/701,076, filed on Sep. 30, 2024.

(51) Int. Cl. C12N 5/00; C07K 7/64; C12M 1/12; C12M 3/00; C12N 5/0793

(52) U.S. Cl. CPC C12N 5/0062; C07K 7/64; C12M 21/08; C12M 25/14; C12N 5/0619; C12N 2533/50

(57) ABSTRACT

Provided herein are netrin-1 mimetic peptide amphiphiles (PAs) comprising a netrin-1 mimetic sequence, nanofibers displaying the bioactive PAs, and methods of use thereof. The disclosed PAs may be used in cell culture methods and in methods of treating central nervous system injury.

### PRIORITY STATEMENT

This application claims priority to and the benefit of U.S. Provisional Patent Application No. 63/701,076, filed Sep. 30, 2024, the entire contents of which are incorporated herein by reference for all purposes.

### STATEMENT REGARDING FEDERALLY SPONSORED RESEARCH OR DEVELOPMENT

This invention was made with government support under grants NS104219 and EB003806 awarded by The National Institutes of Health. The government has certain rights in the invention.

### SEQUENCE LISTING

The text of the computer readable sequence listing filed herewith, titled “NWEST-43696-202_SQL.xml”, created Sep. 25, 2025, having a file size of 16,511 bytes, is hereby incorporated by reference in its entirety.

### FIELD

Provided herein are peptide amphiphiles (PAs) comprising a netrin-1 mimetic sequence, nanofibers comprising the same, and methods of use thereof. In some embodiments, provided herein are netrin-1 mimetic peptide amphiphiles for use in cell culture methods. In other embodiments, provided herein are netrin-1 mimetic peptide amphiphiles for use in methods of treating nervous system injury.

### BACKGROUND

The central nervous system (CNS) requires a complex combination of proteins, including brain-derived neurotrophic factor (BDNF), glial cell-derived neurotrophic factor (GDNF), and neurotrophin-3 (NT-3), among others, to support neuronal maturation, sprouting, guidance, and health throughout development and adulthood. Following CNS injury, delivery of these factors can also support the survival and growth of neurons. The development of the glial scar, an anatomic barrier formed around the lesion site composed of reactive astrocytes, extracellular matrix proteins, and chemorepellent factors, effectively contains the injury and protects adjacent uninjured tissue. However, the glial scar also prevents neurons from extending their processes back through the lesion site over time, preventing the restoration of important neural pathways and resulting in chronic nervous system dysfunction. Recent studies have shown that the application of neurotrophic factors following CNS injury can support neuron growth, despite the presence of the glial scar. Unfortunately, the short half-life of proteins in vivo is a major limitation for their use as CNS therapies. As such, what is needed are improved methods for supporting neuronal growth, including following CNS injury.

### SUMMARY

In some aspects, provided herein are netrin-1 mimetic peptide amphiphiles. In some embodiments, provided herein is a netrin-1 mimetic peptide amphiphile comprising a hydrophobic tail, a structural peptide segment, a charged peptide segment, and a netrin-1 mimetic sequence.

In some embodiments, the hydrophobic tail is a non-peptide hydrophobic segment. In some embodiments, the hydrophobic tail comprises an 8-24 carbon alkyl chain (C₈₋₂₄). For example, in some embodiments the hydrophobic tail comprises a 16 carbon alkyl chain (C₁₆).

In some embodiments, the structural peptide segment has a propensity for forming β-sheet conformations. In some embodiments, the structural peptide segment comprises 2 to 8 non-polar residues. For example, in some embodiments, the structural peptide segment comprises V₂A₂(SEQ ID NO: 2) or A₂V₂(SEQ ID NO: 5).

In some embodiments, the charged peptide segment comprises an acidic, basic, or zwitterionic peptide segment. For example, in some embodiments, charged peptide segment comprises EE, EEE, or EEEE (SEQ ID NO: 4) or KK, KKK, or KKKK (SEQ ID NO: 12).

In some embodiments, the netrin-1 mimetic sequence comprises IDP. In some embodiments, the netrin-mimetic sequence is a cyclic peptide. For example, in some embodiments, the netrin-1 mimetic sequence is a cyclic peptide comprising the sequence IDP flanked by suitable residues to promote cyclization. In some embodiments, the flanking residues comprise cysteine residues. In some embodiments, the netrin-1 mimetic peptide sequence comprises CIDPC (SEQ ID NO: 1). In some embodiments, the sequence CIDPC (SEQ ID NO: 1) is cyclized via disulfide bonding (e.g. forming a disulfide bridge) between the two cysteine residues (e.g. the flanking cysteine residues). In some embodiments, the flanking residues comprise glutamic acid or aspartic acid residues on one end of the IDP sequence, and lysine residues on the other end of the IDP sequence. For example, in some embodiments the netrin-1 mimetic peptide sequence comprises EIDPK (SEQ ID NO: 14) or DIDPK (SEQ ID NO: 15). Such an exemplary netrin-1 mimetic peptide is cyclized by formation of a lactam bridge. In some embodiments, the netrin-1 mimetic peptide is cyclized by formation of lactone and thiolactone bridges (e.g. between flanking amino acids containing carboxyl, hydroxyl, or mercapto functional groups). In some embodiments, the netrin-1 mimetic peptide is cyclized by formation of thioether or ether bridges (e.g. between flanking amino acids containing hydroxyl or mercapto functional groups).

In some embodiments, the netrin-1 mimetic sequence is attached to the charged peptide segment by a linker. In some embodiments, the linker is a peptide linker. For example, in some embodiments the linker comprises GG.

In some embodiments, the peptide amphiphile comprises C₈₋₂₄-V₂A₂E₂G₂CIDPC (SEQ ID NO: 11).

In some aspects, provided herein are nanofibers comprising the netrin-1 mimetic peptide amphiphiles described herein.

In some embodiments, the nanofiber further comprises one or more filler peptide amphiphiles, wherein the filler peptide amphiphiles comprise a hydrophobic tail, a structural peptide segment, and a charged peptide segment, and wherein the filler peptide amphiphiles do not comprise the netrin-1 mimetic sequence.

In some embodiments, the nanofiber comprises 10-40% netrin-1 mimetic peptide amphiphiles and 60-90% filler peptide amphiphiles In some embodiments, the nanofiber comprises about 30% netrin-1 mimetic peptide amphiphiles and about 70% filler peptide amphiphiles.

The peptide amphiphiles and nanofibers provided herein may be formulated into a pharmaceutical composition. In some aspects, provided herein is a pharmaceutical composition comprising a peptide amphiphile or a nanofiber described herein.

The peptide amphiphiles, nanofibers, and compositions (e.g. pharmaceutical compositions) provided herein find use in methods of treating nervous system injury or disease in a subject. In some aspects, provided herein is a method of treating a nervous system injury or disease in a subject in need thereof comprising administering a therapeutically effective amount of a pharmaceutical composition provided herein to the subject. The nervous system injury or disease may be a central nervous system injury or disease. In some embodiments, the disease is characterized by a lack of netrin-1 upregulation. For example, in some embodiments the disease is Alzheimer's disease, Parkinson's disease, or stroke.

In some aspects, provided herein are scaffolds (e.g. hydrogels) comprising a nanofiber provided herein. Such scaffolds find use in cell/organoid culture methods.

In some embodiments, provided herein are methods of culturing cells or organoids, comprising contacting the cells or the organoid with a scaffold provided herein.

In some aspects, provided herein are systems comprising a scaffold and a cell or an organoid cultured on the scaffold. Such systems may be provided to a subject for treatment of a nervous system injury or disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1I show experimental and computational analysis of netrin-1 mimetic peptide amphiphile (N1-PA) assemblies. (FIG. 1A) Chemical structure of E₂-PA showing glutamic acid residues in dark gray. (FIG. 1B) Chemical structure of N1-PA showing glutamic acid residues in dark gray, glycine linker residues in black, and the cyclic CIDPC (SEQ ID NO: 1) sequence in green. (FIG. 1C) Molecular graphics rendering of a co-assembly containing 15% N1-PA and 85% E₂-PA molecules. (FIG. 1D) Prediction from CG-MD simulations of the relative percent of N1-PA molecules incorporated in fibers vs free micellar aggregates and nonincorporated after 10 μs. (FIG. 1E) Snapshots of the CG-MD simulations for assemblies of different N1-PA compositions after 10 μs. Molecules are shown as colored in panels A and B, the simulation box is also shown in green, and water and ions have been removed for clarity; 60% and 90% compositions show only the C₁₆ segment of N1-PA molecules that were not incorporated into fibrils. (FIG. 1F) Transmission electron microscopy images of N1-PA co-assembled with E₂-PA. Scale bars are 200 nm. (FIG. 1G) Small-angle X-ray scattering of N1-PA assemblies at varying molar ratios. The slopes were obtained in the Guinier region between q values of 0.005 and 0.01 in the log-log plot for fibers and between q values of 0.015 and 0.03 for systems that formed irregular aggregates (intensities were offset at y-axis for clarity). (FIG. 1H) Wide-angle X-ray scattering of N1-PA assemblies. (FIG. 1I) Fourier transform infrared spectra of N1-PA assemblies (the absorbances were offset at the y-axis for clarity). A significant peak is indicated at 1613 cm-1 by a dotted line.

FIGS. 2A-2F show N1-PA activates DCC receptor-related pathways. (FIG. 2A) Representative Western blot of PI3K, p-PLCγ, pERK1/2, ERK1/2, and Rac1/2 in primary neurons treated with 1 μM peptide or PA in solution for 24 h; assemblies contained 15% N1-PA or L-N1-PA monomer. Positive and negative controls are also shown, corresponding to recombinant mouse netrin-1 protein (rN1) at a concentration of 250 ng/mL and untreated media, respectively. (FIG. 2B) Densitometry analysis from Western blot data corresponding to PI3K, Rac1/2, p-PLCγ, and pERK1/2. (FIG. 2C) Representative confocal images of primary neurons treated with 1 μM peptide or PA with and without the addition of the anti-DCC receptor antibody; neurons were stained with MAP2 (red), SMI312 (green), and DAPI (blue). Positive and negative controls are also shown, corresponding to recombinant mouse netrin-1 protein (rN1) at a concentration of 250 ng/mL and untreated media, respectively. Assemblies contained 15% of the N1-PA or L-N1-PA monomer. (FIG. 2D) Quantification of the average length of SMI312-positive primary axons of primary neurons treated with 1 μM PAs in solution for 24 h. (FIG. 2E) Representative Western blot of PI3K, p-PLCγ, pERK1/2, ERK1/2, and Rac1/2 in primary neurons treated with 1 μM peptide or PA with and without the addition of the anti-DCC receptor antibody. Positive and negative controls are also shown, corresponding to recombinant mouse netrin-1 protein (rN1) at a concentration of 250 ng/mL and untreated media, respectively. Assemblies contained 15% N1-PA or L-N1-PA monomer. (FIG. 2F) Densitometry analysis from Western blot data corresponding to PI3K, pPLCγ, pERK1/2, and Rac1/2 with and without the addition of the anti-DCC receptor antibody. For panel C, one-way ANOVA with multiple comparisons was performed with α=0.05 (* vs control, # vs rN1, \hat{ } vs peptide): (*) P<0.05, (**) P<0.01, (***) P<0.001, (****) P<0.0001. For panel D, two-way ANOVA with multiple comparisons was performed with α=0.05: (*) P<0.05, (**) P<0.01, (***) P<0.001, (****) P<0.0001. For panel F, one-way ANOVA with multiple comparisons was performed with α=0.05: (*) P<0.05, (**) P<0.01, (***) P<0.001, (****) P<0.0001.

FIGS. 3A-3G show measuring neurite outgrowth with microfluidics devices. (FIG. 3A) Rendering of a XonaChip microfluidics two-compartment device; the blue compartment contains the soma of neurons, and the green one contains the treatment. (FIG. 3B) Depiction of microchannels that separate the neuron compartment from the treatment compartment. (FIG. 3C) Image of neurites of primary neurons exiting microchannels into the treatment compartment after 1 week; neurons are stained for Tuj1 (red), SMI312 (green), and DAPI (blue) (PA is also nonspecifically stained by DAPI). (FIG. 3D) Representative confocal images of Tuj1 (red) stained neurites exiting the microchannels into the treatment compartment after 1 week (a positive control of 250 ng/mL recombinant netrin-1 protein (rN1) was used). 1 μM treatments of peptide, 15% N1-PA, and E₂-PA were used. (FIG. 3E) Schematic of how confocal images from microfluidics devices were analyzed; starting at the edge of the microchannels in the treatment chamber (0 μm), the normalized integrated density of fluorescent pixels of Tuj1 (red) was measured in rectangles 20 μm in width spanning the entire length of the treatment chamber. (FIG. 3F) Plot of all normalized integrated density of fluorescent pixel values obtained from microfluidics device analysis (a one-way ANOVA was performed between the average of each condition to determine statistical significance). (FIG. 3G) Quantification of normalized Tuj1 (red) integrated density of fluorescent pixel values at regular distances from the exit of the microchannels. For panels E and G, one-way ANOVA with multiple comparisons was performed on the average of all data points within each condition with α=0.05: (*) P<0.05, (**) P<0.01, (***) P<0.001, (****) P<0.0001.

FIGS. 4A-4H show N1-PA influences neuron synaptogenesis and functional electrical activity. (FIG. 4A) Representative confocal images of primary neurons cultured for 1 week (a positive control of 250 ng/mL recombinant netrin-1 (rN1) and 1 μM peptide or PA treatments of E₂-PA and 15% N1-PA, respectively, were used). (FIG. 4B) Representative Western blot of PSD95, Tuj1, and Syp in primary neurons after 2 weeks of treatment (a positive control of 250 ng/mL recombinant netrin-1 (rN1) and 1 μM peptide or PA treatments of E₂-PA, 15% N1-PA, and 15% L-N1-PA, respectively, were used). (FIG. 4C) Densitometry analysis of PSD95, Tuj1, and Syp. (FIG. 4D) Representative confocal images of neurites labeled with Tuj1 (white), PSD95 (green), and synaptophysin (red) after 12 days (a positive control of 250 ng/mL recombinant netrin-1 (rN1) and 1 μM PA treatments of E₂-PA and 15% N1-PA were used). (FIG. 4E) Representative brightfield image of a multielectrode array plate well with primary cortical neurons growing on its surface. (FIG. 4F) Representative snapshots of the distribution of spike amplitudes across treated primary neurons plated in an MEA plate well at 21 days. (FIG. 4G) Number of spikes per burst and (FIG. 4H) synchrony index of primary neurons treated for 21 days (measurements were collected with an MEA plate and averaged over measurements collected for 5 min at 21 days). A 250 ng/mL recombinant netrin-1 positive control (rN1) and 1 μM peptide or PA treatments of E₂-PA and 15% N1-PA, respectively, were used for all electrophysiology measurements. For panel C, one-way ANOVA with multiple comparisons was performed with α=0.05 (* vs control, # vs rN1, A vs peptide): (*) P<0.05, (**) P<0.01, (***) P<0.001, (****) P<0.0001. For panel G, one-way ANOVA with a Kruskal-Wallis multiple comparisons test was performed with respect to rN1 with α=0.05 (* vs control, # vs rN1, A vs peptide): (*) P<0.05, (**) P<0.01, (***) P<0.001, (****) P<0.0001. For panel H, one-way ANOVA with multiple comparisons was performed in respect to rN1 with α=0.05 (* vs control, # vs rN1, A vs peptide): (*) P<0.05, (**) P<0.01, (***) P<0.001, (****) P<0.0001.

FIG. 5 shows the chemical structure of CIDPC (SEQ ID NO: 1) cyclic peptide.

FIG. 6A-6B show linear netrin-1 PA (L-N1-PA) structure. (FIG. 6A) Chemical structure of L-N1-PA. (FIG. 6B) Molecular graphics rendering of a co-assembly containing 15% L-N1-PA and 85% E2-PA molecules. Glutamic acid residues are in dark grey, glycine linker residues are in black, and the linear SIDPS is in blue.

FIGS. 7A-7D show CG-MD Simulation Snapshots of Different N1-PA Compositions with E2-PA Semitransparent. Snapshots of the CG-MD simulations with different N1-PA compositions after 10 s. PAs are colored as in schemes FIGS. 1A and 1B, simulation box is shown in green, and water and ions are removed for clarity. E2-PA is semitransparent to facilitate visualization of the N1-PA, and 60% and 90% show only the C16 of non-incorporated N1-PAs. (FIG. 7A) 15% N1-PA. (FIG. 7B) 30% N1-PA. (FIG. 7C) 60% N1-PA. (FIG. 7D) 90% N1-PA.

FIGS. 8A-8E show summary of systems showing non-proper incorporation of N1-PA into the E2-PA fiber. Snapshots of CG-MD simulations with (FIG. 8A) 30% and (FIG. 8B) 60% N1-PA after 10 s. PAs are colored as in FIG. 1A-B, simulation box is shown in green, and water and ions are removed for clarity. Bottom images show E2-PA semitransparent to facilitate visualization of the N1-PA, and (B) shows only the C16 of non-incorporated N1-PAs. Aggregation Propensity (AP) of the (FIG. 8C) E2-PA and (FIG. 8D) N1-PA for the two mentioned systems, showing that despite the non-proper incorporation, aggregation level has reached an equilibrium. (E) Radial distribution analysis of the 30% system (corresponding to the snapshots in A) with the detailed distribution of the N1-PA components showing that C16 tails can be found through the whole cross-section, and thus they are not properly incorporated into the fiber aliphatic core.

FIGS. 9A-9I show aggregation propensity (AP), water contact maps, radial distribution analysis, average cluster analysis, and hydration analysis of E2-PA and N1-PA assemblies. Aggregation Propensity (AP) of (FIG. 9A) E2-PA and (FIG. 9B) N1-PA, showing the systems reach equilibration within the simulations time. Water contact maps showing hydration of backbone and aliphatic tail beads of (FIG. 9C) E2-PA and (FIG. 9D) N1-PA, showing how additional hydration of each PA changes upon the addition of the co-assembler. Radial distribution analysis of (FIG. 9E) E2-PA, (FIG. 9F) N1-PA, in simulations with different compositions, and (FIG. 9G) detail on the distribution of each part of the N1-PA in the 15% system, revealing the distribution of each component through the fiber cross-section. (FIG. 9H) Average cluster analysis of incorporated and non-incorporated N1-PA, showing that incorporated clusters size drops when non-incorporated clusters reach a critical size of ˜60. (FIG. 9I) Hydration analysis at different N1-PA percentages to reveal the initial loss of E2-PA hydration and increment at 90%, which could suggest a disruptive effect at these N1-PA loads. The hydration loss is relatively low, as observed in other highly clustered co-assemblies.^{[1]}

FIGS. 10A-10D show structural and intermolecular analysis of linear netrin-1 PA (L-N1-PA) assemblies. (FIG. 10A) Transmission electron microscopy images of increasing concentrations of L-N1-PA co-assembled with E2-PA. (FIG. 10B) Small-angle X-ray scattering of L-N1-PA assemblies at varying molar ratios. The slopes were obtained in the Guinier region between q values of 0.005 and 0.01 in the log-log plot. Intensities were offset at y-axis for clarity. (FIG. 10C) Wide-angle X-ray scattering of L-N1-PA assemblies. (FIG. 10D) Fourier transform infrared (FT-IR) spectroscopy of L-N1-PA assemblies. The absorbances were offset at y-axis for clarity. A significant peak is indicated at 1613 cm⁻¹ by a dotted line.

FIG. 11 shows dynamic light scattering (DLS) of 100% N1-PA. Dynamic light scattering (DLS) of 100% N1-PA micelles in solution.

FIGS. 12A-12D show disulfide bond preservation studies in N1-PA. (FIG. 12A) Chemical structure of uncyclized N1-PA. (FIG. 12B) Schematic of Ellman's reagent reaction with thiols. In the presence of free thiols, Ellman's reagent (DTNB²⁻) undergoes a thiol-disulfide exchange reaction which results in the formation of the yellow TNB²⁻ compound. (FIG. 12C) Qualitative evaluation of color change after adding Ellman's reagent to 15% N1-PA, 100% N1-PA, and 100% uncyclized N1-PA (FIG. 12D) Absorption measurements 15 minutes after the addition of Ellman's reagent to PA solutions.

FIG. 13A-13B show disulfide bond preservation of N1-PA in the presence of glutathione. Liquid chromatograph—mass spectrometry (LC-MS) of 500 M solutions of 100% N1-PA prepared in 10 mM PBS (FIG. 13A) prior to the addition of glutathione (GSH) and (FIG. 13B) 48 hours after the addition of 5 mM GSH.

FIGS. 14A-14D show N1-PA nanofibers interacting with neuron and lactate dehydrogenase (LDH) assays of N1-PA treated neurons for biocompatibility. (FIG. 14A) (top) N1-PA interfacing with a neuron 24 h after treatment as observed in a confocal microscopy obtained image stained with MAP2 (red), SMI312 (green) and DAPI (blue). Confocal image was rendered with IMARIS software. White arrows indicate areas of PA fibers non-specifically stained with DAPI. (bottom) Scanning electron microscopy (SEM) image of N1-PA (indicated by white arrow) present in neuron culture 24 h after treatment. Lactate dehydrogenase (LDH) present in neuron culture media (FIG. 14B) 24 hours, (FIG. 14C) 72 hours, and (FIG. 14D) 1 week after treatment (negative and positive controls are also shown corresponding to untreated media (to evaluate base-level cell survival) and a lysis buffer (to evaluate a maximum value of LDH), respectively.

FIGS. 15A-15B show cell viability of treated primary cortical neurons. (FIG. 15A) Representative fluorescent images of calcein (green) and propidium iodide (red) stained primary neurons 72 h after treatment with 1 μM peptide or PA with assemblies containing 15% N1-PA or L-N1-PA. Calcein and propidium iodide label live and dead cells, respectively. Recombinant mouse netrin-1 protein (rN1) was applied at a concentration of 250 ng/mL. A lysis buffer treatment was used for the dead control. (FIG. 15B) Quantification of the ratio of the number of live (calcein labelled) cells compared to the sum of all live (calcein labelled) and dead (PI labelled) cells. For (B) one-way ANOVA with a Tukey's multiple comparisons test was performed with α=0.05: (*) P<0.05, (**) P<0.01, (***) P<0.001, (****) P<0.0001.

FIG. 16A-16B show DCC-receptor pathway activation of neurons treated with 1 μM PA for 24 h. (FIG. 16A) Representative Western blot of SMI312, PI3K, Rac1/2, p-PLCγ, pERK1/2, and ERK1/2 in neurons treated with 1 μM of PA for 24 h; positive and negative controls are also shown corresponding to recombinant mouse netrin-1 protein (rN1) (at concentrations of 250 ng/mL and 500 ng/mL) and to untreated media, respectively. (FIG. 16B) Densitometry analysis of SMI312, PI3K, Rac1/2, p-PLCγ, and pERK1/2 treated with 1 μM PA. For (B) one-way ANOVA with a Bonferroni's multiple comparisons test was performed with α=0.1 for SMI312. One-way ANOVA with a Tukey's multiple comparisons test was performed with α=0.05 for PI3K, Rac1/2, p-PLCγ, and pERK1/2 (* vs. Control, # vs. 250 ng/mL rN1, o vs. 500 ng/mL rN1, \hat{ } vs. peptide): (*) P<0.05, (**) P<0.01, (***) P<0.001, (****) P<0.0001.

FIG. 17A-17F show DCC-receptor pathway activation of neurons treated with 1 μM PA for 24 h. (FIG. 17A) Representative Western blot of SMI312, PI3K, Rac1/2, p-PLCγ, pERK1/2, and ERK1/2 in neurons treated for 24 h with 1 μM PA treatments; positive and negative controls are also shown corresponding to recombinant mouse netrin-1 protein (rN1) (at concentrations of 250 ng/mL and 500 ng/mL) and to untreated media, respectively. Densitometry analysis of (FIG. 17B) SMI312, (FIG. 17C) PI3K, (FIG. 17D) Rac1/2, (FIG. 17E) p-PLCγ, and (FIG. 17F) pERK1/2. For (B-F) one-way ANOVA with a Tukey's multiple comparisons test was performed with α=0.05 (* vs. Control, # vs. 250 ng/mL rN1, \hat{ } vs. 500 ng/mL rN1, \hat{ } vs. peptide): (*) P<0.05, (**) P<0.01, (***) P<0.001, (****) P<0.0001.

FIG. 18A-18E show comparison of DCC-receptor pathway activation between 15 and 30% N1-PA at various concentrations. (FIG. 18A) Representative Western blot of PI3K, p-PLCγ, pERK1/2, ERK1/2, and Rac1/2 in neurons treated for 24 h with a range of PA concentrations; positive and negative controls are also shown corresponding to recombinant mouse netrin-1 protein (rN1) (at concentrations of 250 ng/mL and 500 ng/mL) and to untreated media, respectively. Densitometry analysis of (FIG. 18B) PI3K, (FIG. 18C) p-PLCγ, (FIG. 18D) pERK1/2, and (FIG. 18E) Rac1/2. For (B-E) one-way ANOVA with a Tukey's multiple comparisons test was performed with α=0.05 (* vs. Control): (*) P<0.05, (**) P<0.01, (***) P<0.001, (****) P<0.0001.

FIG. 19 shows morphology of neurons treated with 1 μM N1-PA assemblies with and without anti-DCC receptor antibody for 24 h. Representative confocal images of neural cells treated with 1 μM PA treatments with and without the addition of anti-DCC receptor antibody. Neurons were stained with MAP2 (red), SMI312 (green), and DAPI (blue); a positive control is also shown corresponding to recombinant mouse netrin-1 protein (rN1) at a concentration of 500 ng/mL.

FIG. 20 shows morphology of neurons treated with 1 μM N1-PA assemblies with and without anti-DCC receptor antibody for 24 h. Representative confocal images of neural cells treated with 1 μM peptide or PA treatments with and without the addition of anti-DCC receptor antibody. Neurons were stained with MAP2 (red), SMI312 (green), and DAPI (blue).

FIG. 21 shows morphology of neurons treated with 1 μM N1-PA assemblies with and without anti-DCC receptor antibody for 24 h. Representative confocal images of neural cells treated with 1 μM peptide or PA treatments with and without the addition of anti-DCC receptor antibody. Neurons were stained with MAP2 (red), SMI312 (green), and DAPI (blue).

FIG. 22A-22E show DCC-receptor pathway activation of neurons treated with 1 μM PA for 24 h. (FIG. 22A) Representative Western blot of PI3K, p-PLCγ, pERK1/2, ERK1/2, and Rac1/2 in neurons treated for 24 h with 1 μM PA treatments; positive and negative controls are also shown corresponding to recombinant mouse netrin-1 protein (rN1) (at concentrations of 250 ng/mL and 500 ng/mL) and to untreated media, respectively. Densitometry analysis of (FIG. 22B) PI3K, (FIG. 22C) p-PLCγ, (FIG. 22D) pERK1/2, and (FIG. 22E) Rac1/2. For (B-E) one-way ANOVA with a Tukey's multiple comparisons test was performed with α=0.05 (* vs. Control, # vs. 250 ng/mL rN1, o vs. 500 ng/mL rN1, \hat{ } vs. peptide): (*) P<0.05, (**) P<0.01, (***) P<0.001, (****) P<0.0001.

FIGS. 23A-23B show axon network analysis of neurons treated with 1 μM PA for 72 h. (FIG. 23A) Representative confocal images of neural cells treated with 1 μM peptide or PA treatments with and without the addition of anti-DCC receptor antibody for 72 h. Neurons were stained with MAP2 (red), SMI312 (green), and DAPI (blue); positive and negative controls are also shown corresponding to recombinant mouse netrin-1 protein (rN1) (at concentrations of 250 ng/mL and 500 ng/mL) and to untreated media, respectively. (FIG. 23B) Quantification of the average area of SMI312 positive axons for cells treated with 1 μM treatments and 250 ng/mL recombinant netrin-1 protein (Netrin-1). For (B) two-way ANOVA with a Tukey's multiple comparisons test was performed with α=0.05: (*) P<0.05, (**) P<0.01, (***) P<0.001, (****) P<0.0001.

FIG. 24 shows Axon network of neurons treated with 7 μM PA for 72 h. Representative confocal images of neural cells treated with 7 μM peptide or PA treatments with and without the addition of anti-DCC receptor antibody. Neurons were stained with MAP2 (red), SMI312 (green), and DAPI (blue).

FIGS. 25A-25B show SMI312 positive neurite growth and analysis schematic of XonaChip® microfluidics devices. (FIG. 25A) Representative confocal images of SMI312 (green) stained axons exiting the Xonachip® microchannels into the treatment compartment after 1 week; positive and negative controls are also shown corresponding to recombinant mouse netrin-1 protein (rN1) at a concentration of 250 ng/mL and to untreated media, respectively. (FIG. 25B) Schematic of how confocal images of microfluidics devices were analyzed. Starting at the edge of the microchannels in the treatment chamber (0 μm), the normalized integrated density of fluorescent pixels of Tuj1 (red) was measured in rectangles 20 μm in width spanning the length of the treatment chamber.

FIG. 26 shows average area of MAP2 present in neuron cultures after 1 week of treatment. Quantification of the average area of MAP2 (green) present per field of vision of neurons treated with 1 μM peptide or PA after 1 week; positive and negative controls are also shown corresponding to recombinant mouse netrin-1 protein (rN1) (at a concentration of 250 ng/mL) and to untreated media, respectively. One-way ANOVA with a Tukey's multiple comparisons test was performed with α=0.05: (*) P<0.05, (**) P<0.01, (***) P<0.001, (****) P<0.0001.

FIGS. 27A-27C show neuron SMI312 and MAP2 expression after 2 weeks of treatment. (FIG. 27A) Representative Western blot of SMI312 and MAP2 in neurons treated with 1 μM PA in solution for 2 weeks; positive and negative controls are also shown corresponding to recombinant mouse netrin-1 protein (rN1) (at a concentration of 250 ng/mL) and to untreated media, respectively. Densitometry analysis of (B) SMI312 and (C) MAP2. For (FIG. 27B-FIG. 27C) one-way ANOVA with a Tukey's multiple comparisons test was performed with α=0.05 (* vs. Control, # vs. rN1, \hat{ } vs. Peptide): (*) P<0.05, (**) P<0.01, (***) P<0.001, (****) P<0.0001.

FIGS. 28A-28D show multielectrode array (MEA) plate experimental design and mean firing rate, burst frequency, and inter-burst interval measurements. (FIG. 28A) PA treatment and recording timeline for neurons cultured on multielectrode array (MEA) plates over 21 days. Analysis of recordings obtained 21 days from neurons treated with 1 μM PA for (FIG. 28B) mean firing rate, (FIG. 28C) burst frequency, and (FIG. 28D) inter-burst interval. For (B-F) one-way ANOVA with a Tukey's multiple comparisons test was performed relative to rN1 with α=0.05: (*) P<0.05, (**) P<0.01, (***) P<0.001, (****) P<0.0001.

FIG. 29A-29C show severe spinal cord contusion model and PA treatment effect on the inflammatory response. (FIG. 29A) Timeline of severe spinal cord contusion mouse model. CD1 mice were contused at the T10-11 spinal cord level using an impactor delivering 85 kDynes of force with a dwell time of 60 s. 24 hrs after contusion, PA was delivered via stereotactic injection into the lesion site. Mice were evaluated for hind limb recovery once a week for 3 months, after which spinal cord tissue was harvested for histological evaluation and analysis. (FIG. 29B) Representative confocal images of spinal cord tissue 3 months after injury treated with E₂-PA, N1-PA, or saline (sham). Tissue was stained with GFAP (red), Iba-1 (green), and DAPI (blue). (FIG. 29C) Number of microglia present per mm² in representative areas surrounding the lesion site. (FIG. 29D) Representative dot blot of Iba-1, F4/80, GFAP, and total protein (TP) in injured spinal cord tissue treated with saline (sham), E₂-PA, or N1-PA. Uninjured spinal cord tissue is also included as a positive control. For (C) a one-way ANOVA with multiple comparisons was performed with α=0.05: (*) P<0.05, (**) P<0.01, (***) P<0.001, (****) P<0.0001.

FIG. 30A-30C show lesion length and glial scarring affected by PA treatment following SCI. (FIG. 30A) Representative confocal images of transverse sections of spinal cord 3 months after injury treated with E₂-PA, N1-PA, or saline (sham). Tissue was stained with GFAP (red) and DAPI (blue). (FIG. 30B) Quantification of the lesion length 3 months after injury in mice treated with saline (sham), E₂-PA, or N1-PA. The lesion length is considered the distance between the proximal and distal border of injury site as indicated by the astrocytic border. For (B) a one-way ANOVA with a Tukey's multiple comparisons test was performed with α=0.05: (*) P<0.05, (**) P<0.01, (***) P<0.001, (****) P<0.0001. (FIG. 30C) Representative dot blot of Iba-1, F4/80, GFAP, and total protein (TP) in injured spinal cord tissue treated with saline (sham), E₂-PA, OC-E₂-PA, N1-PA, or OC-N1-PA. Uninjured spinal cord tissue is also included as a positive control.

FIG. 31A-31C show neurite regrowth into the lesion site. (FIG. 31A) Representative confocal images of transverse sections of spinal cord 3 months after injury treated with saline (sham), E₂-PA, or N1-PA. Tissue was stained with S100B (green), NF (red), and DAPI (blue). (FIG. 31B) Percent neurofilament (NF) area measured at distances across the lesion site, beginning at the proximal border and ending at the distal border. (FIG. 31C) Percent neurofilament (NF) measured within the entire lesion site. For (C) a one-way ANOVA with a Tukey's multiple comparisons test was performed with α=0.05: (*) P<0.05, (**) P<0.01, (***) P<0.001, (****) P<0.0001.

FIG. 32. N1-PA containing PA treatments reduce tissue degeneration distal to SCI. Representative confocal images of transverse sections of spinal cord 3 months after injury treated with E₂-PA or N1-PA. Tissue was stained with GFAP (red), Tuj1 (green), and DAPI (blue).

FIGS. 33A-33B show PA treatment reduces neuron degeneration caudal to lesion site. (FIG. 33A) Representative confocal images of transverse sections of spinal cord 3 months after injury treated with E₂-PA or N1-PA. Tissue was stained with laminin (red), Tuj1 (green), and DAPI (blue). (FIG. 33B) Total number of Tuj1-positive neurons present within each labelled region distal to the lesion site. Region 1 indicates the region closest to lesion site. Region 3 indicates the region furthest from the lesion site. For (B) a two-way ANOVA with a Tukey's multiple comparisons test was performed with α=0.05: (*) P<0.05, (**) P<0.01, (***) P<0.001, (****) P<0.0001.

FIG. 34. Functional recovery following severe spinal cord contusion. (FIG. 34A) Schematic illustrating Basso mouse scale (BMS) scoring of hind limb functional recovery. Blinded BMS scoring was performed before the contusion (Week 0), immediately after contusion (Week 1), and then once a week for the duration of the study. (FIG. 34B) Representative images of hind limb positioning of saline (sham), E₂-PA, or N1-PA treated mice 3 months post-SCI. Solid black lines indicate positioning and angle of hind limbs. (FIG. 34C) BMS scores for saline (sham), E₂-PA, or N1-PA treated mice each week. (FIG. 34D) Schematic of lesion site following severe spinal cord contusion. Following contusion, neurites (blue) covered in myelin (yellow) are damaged at the site of injury. After 24 hours, PA nanofibers (green) are injected into the lesion. Over the course of the 3-month study, neurites begin to grow into the PA. At the conclusion of 3 months, we hypothesize that neurites in PA treated conditions have significant regrowth into the injury core, supporting functional recovery. For (C) a two-way ANOVA with multiple comparisons was performed with α=0.1. (* in color corresponding to experimental group=experimental group vs. Sham, \hat{ }=E₂-PA vs. I-E₂-PA): (*) P<0.05, (**) P<0.01, (***) P<0.001, (****) P<0.0001.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO: Z (e.g., 100 amino acids) may have up to X substitutions (e.g., 10) relative to SEQ ID NO: Z, and may therefore also be expressed as “having X (e.g., 10) or fewer substitutions relative to SEQ ID NO: Z.”

As used herein, the term “nanofiber” refers to an elongated or threadlike filament (e.g., having a significantly greater length dimension that width or diameter) with a diameter typically less than 100 nanometers.

As used herein, the term “scaffold” refers to a material capable of supporting growth and differentiation of a cell.

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, macromolecules, etc.) and the multicomponent assemblies, complexes, systems, and/or fibers that form as a result.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components.

As used herein, the term “peptide amphiphile” refers to a molecule that, at a minimum, includes a non-peptide lipophilic (hydrophobic) segment, AND a structural peptide segment and/or charged peptide segment (often both). In some embodiments, a peptide amphiphile additionally comprises a bioactive segment, such as a netrin-1 mimetic sequence. The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptide segment (e.g., comprising an acyl group of six or more carbons), (2) a structural peptide segment; (3) a charged peptide segment, and (4) a bioactive segment (e.g., a netrin-1 mimetic sequence).

The term “peptide amphiphile” is inclusive of both “bioactive peptide amphiphiles” and “filler peptide amphiphiles” or “diluent peptide amphiphiles”. A “bioactive peptide amphiphile” refers to a peptide amphiphile comprising a bioactive segment, such as a netrin-1 mimetic sequence. A “netrin-1 mimetic peptide amphiphile” is an example of a “bioactive peptide amphiphile”. In contrast, a filler or diluent peptide amphiphile does not comprise a bioactive segment (e.g. does not comprise a netrin-1 mimetic sequence).

As used herein and in the appended claims, the term “lipophilic moiety” or “hydrophobic moiety” refers to the moiety (e.g., an acyl, ether, sulfonamide, or phosphodiester moiety) disposed on one terminus (e.g., C-terminus, N-terminus) of the peptide amphiphile, and may be herein and elsewhere referred to as the lipophilic or hydrophobic segment or component. The hydrophobic segment should be of a sufficient length to provide amphiphilic behavior and aggregate (or nanosphere or nanofiber) formation in water or another polar solvent system.

Accordingly, in the context of the embodiments described herein, the hydrophobic component preferably comprises a single, linear acyl chain of the formula: Cₙ₋₁H₂ₙ₋₁C(O)—where n=2-25. In some embodiments, a linear acyl chain is the lipophilic group (saturated or unsaturated carbons), palmitic acid. However, other lipophilic groups may be used in place of the acyl chain such as steroids, phospholipids and fluorocarbons.

As used interchangeably herein, the terms “structural peptide” or “structural peptide segment” refer to a portion of a peptide amphiphile, typically disposed between the hydrophobic segment and the charged peptide segment. The structural peptide is generally composed of three to ten amino acid residues with non-polar, uncharged side chains (e.g., His (H), Val (V), Ile (I), Leu (L), Ala (A), Phe (F)) selected for their propensity to form hydrogen bonds or other stabilizing interactions (e.g., hydrophobic interactions, van der Waals' interactions, etc.) with structural peptide segments of adjacent structural peptide segments. In some embodiments, nanofibers of peptide amphiphiles having structural peptide segments display linear or 2D structure when examined by microscopy and/or α-helix and/or j-sheet character when examined by circular dichroism (CD). In some embodiments, the structural peptide segment has a propensity for forming j-sheet conformations. Such a structural peptide segment is also referred to herein as a “beta (β)-sheet-forming peptide segment”. In some embodiments, the structural peptide comprises V₂A₂ (SEQ ID NO: 2), In other embodiments, the structural peptide comprises V₃A₃ (SEQ ID NO: 3).

As used herein, the term “beta (β)-sheet-forming peptide segment” refers to a structural peptide segment that has a propensity to display j-sheet-like character (e.g., when analyzed by CD). In some embodiments, amino acids in a beta (β)-sheet-forming peptide segment are selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety).

As used herein, the term “charged peptide segment” refers to a portion of a peptide amphiphile that is rich (e.g., >50%, >75%, etc.) in charged amino acid residues, or amino acid residue that have a net positive or negative charge under physiologic conditions. A charged peptide segment may be acidic (e.g., negatively charged), basic (e.g., positively charged), or zwitterionic (e.g., having both acidic and basic residues).

As used herein, the terms “carboxy-rich peptide segment,” “acidic peptide segment,” and “negatively-charged peptide segment” refer to a peptide sequence of a peptide amphiphile that comprises one or more amino acid residues that have side chains displaying carboxylic acid side chains (e.g., Glu (E), Asp (D), or non-natural amino acids). A carboxy-rich peptide segment may optionally contain one or more additional (e.g., non-acidic) amino acid residues. Non-natural amino acid residues, or peptidomimetics with acidic side chains could be used, as will be evident to one ordinarily skilled in the art. There may be from about 2 to about 7 amino acids, and or about 3 or 4 amino acids in this segment.

As used herein, the terms “amino-rich peptide segment”, “basic peptide segment,” and “positively-charged peptide segment” refer to a peptide sequence of a peptide amphiphile that comprises one or more amino acid residues that have side chains displaying positively-charged acid side chains (e.g., Arg (R), Lys (K), His (H), or non-natural amino acids, or peptidomimetics). A basic peptide segment may optionally contain one or more additional (e.g., non-basic) amino acid residues. Non-natural amino acid residues with basic side chains could be used, as will be evident to one ordinarily skilled in the art. There may be from about 2 to about 7 amino acids, and or about 3 or 4 amino acids in this segment.

As used herein, the term “bioactive peptide” refers to amino acid sequences that mediate the action of sequences, molecules, or supramolecular complexes associated therewith. Peptide amphiphiles and structures (e.g., nanofibers) bearing bioactive peptides (e.g., a netrin-1 mimetic sequence) exhibit the functionality of the bioactive peptide.

As used herein, the term “biocompatible” refers to materials and agents that are not toxic to cells or organisms. In some embodiments, a substance is considered to be “biocompatible” if its addition to cells in vitro results in less than or equal to approximately 10% cell death, usually less than 5%, more usually less than 1%.

As used herein, “biodegradable” as used to describe the polymers, hydrogels, and/or wound dressings herein refers to compositions degraded or otherwise “broken down” under exposure to physiological conditions. In some embodiments, a biodegradable substance is a broken down by cellular machinery, enzymatic degradation, chemical processes, hydrolysis, etc. In some embodiments, a wound dressing or coating comprises hydrolyzable ester linkages that provide the biodegradability.

As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

As used herein, the terms “treat,” “treatment,” and “treating” refer to reducing the amount or severity of a particular condition, disease state (e.g., CNS injury), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the terms “prevent,” “prevention,” and preventing” refer to reducing the likelihood of a particular condition or disease state (e.g., CNS injury) from occurring in a subject not presently experiencing or afflicted with the condition or disease state. The terms do not necessarily indicate complete or absolute prevention. For example “preventing CNS injury” refers to reducing the likelihood of CNS injury occurring in a subject not presently experiencing or diagnosed with a CNS injury. In order to “prevent CNS injury” a composition or method need only reduce the likelihood of CNS injury, not completely block any possibility thereof. “Prevention,” encompasses any administration or application of a therapeutic or technique to reduce the likelihood of a disease developing (e.g., in a mammal, including a human). Such a likelihood may be assessed for a population or for an individual.

As used herein, the terms “co-administration” and “co-administering” refer to the administration of at least two agent(s) or therapies to a subject (e.g., a PA nanofiber and one or more therapeutic agents). In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### DETAILED DESCRIPTION

Provided herein are peptide amphiphiles (PAs) comprising a netrin-1 mimetic sequence, nanofibers displaying the bioactive PAs, and methods of use thereof.

In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus (or C-terminus) of the peptide, in order to create the lipophilic segment (although in some embodiments, alignment of nanofibers is performed via techniques not previously disclosed or used in the art (e.g., extrusion through a mesh screen). Synthesis typically starts from the C-terminus, to which amino acids are sequentially added using either a Rink amide resin (resulting in an —NH2 group at the C-terminus of the peptide after cleavage from the resin), or a Wang resin (resulting in an —OH group at the C-terminus). Accordingly, some embodiments described herein encompass peptide amphiphiles having a C-terminal moiety that may be selected from the group consisting of —H, —OH, —COOH, —CONH2, and —NH2.

In some embodiments, peptide amphiphiles comprise a hydrophobic segment (i.e. a hydrophobic tail) linked to a peptide. In some embodiments, the peptide comprises a structural peptide segment. In some embodiments, the structural peptide segment is a hydrogen-bond-forming segment, or beta-sheet-forming segment. In some embodiments, the structural peptide segment has the propensity to form random coil structures (e.g. a total propensity for forming β-sheet conformations of 4 or less). In some embodiments, the peptide comprises a charged segment (e.g., acidic segment, basic segment, zwitterionic segment, etc.). In some embodiments, the peptide further comprises linker or spacer segments for adding solubility, flexibility, distance between segments, etc. In some embodiments, peptide amphiphiles comprise a spacer segment (e.g., peptide and/or non-peptide spacer) at the opposite terminus of the peptide from the hydrophobic segment. In some embodiments, the spacer segment comprises peptide and/or non-peptide elements. In some embodiments, the spacer segment comprises one or more bioactive groups (e.g., alkene, alkyne, azide, thiol, etc.). In some embodiments, various segments may be connected by linker segments (e.g., peptide (e.g., GG) or non-peptide (e.g., alkyl, OEG, PEG, etc.) linkers).

The lipophilic or hydrophobic segment is typically incorporated at the N- or C-terminus of the peptide after the last amino acid coupling, and is composed of a fatty acid or other acid that is linked to the N- or C-terminal amino acid through an acyl bond. In aqueous solutions, PA molecules self-assemble (e.g., into cylindrical micelles (a.k.a., nanofibers)) to bury the lipophilic segment in their core and display the bioactive peptide (e.g. netrin-1 mimetic sequence) on the surface. In some embodiments, the structural peptide undergoes intermolecular hydrogen bonding to form beta sheets that orient parallel to the long axis of the micelle. In some embodiments, the structural peptide displays weak intermolecular hydrogen bonding, resulting in a less rigid beta-sheet conformation within the nanofibers.

In some embodiments, compositions described herein comprise PA building blocks that in turn comprise a hydrophobic segment (also referred to as a hydrophobic tail) and a peptide segment (e.g. a structural peptide segment and/or a charged peptide segment). In certain embodiments, a hydrophobic (e.g., hydrocarbon and/or alkyl/alkenyl/alkynyl tail, or steroid such as cholesterol) segment of sufficient length (e.g., 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, 30 carbons or more, or any ranges there between.) is covalently coupled to peptide segment (e.g., a peptide comprising a segment having a preference for beta-strand conformations or other supramolecular interactions) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture.

In some embodiments, to induce self-assembly of an aqueous solution of peptide amphiphiles, the pH of the solution may be changed (raised or lowered) or multivalent ions, such as calcium, or charged polymers or other macromolecules may be added to the solution.

In some embodiments, the hydrophobic segment is a non-peptide segment (e.g., alkyl/alkenyl/alkynyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, heterocyclic rings, aromatic segments, pi-conjugated segments, cycloalkyls, oligothiophenes etc. In some embodiments, the hydrophobic segment comprises an acyl/ether chain (e.g., saturated) of 2-30 carbons (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30).

In some embodiments, PAs comprise one or more peptide segments. Peptide segment may comprise natural amino acids, modified amino acids, unnatural amino acids, amino acid analogs, peptidomimetics, or combinations thereof. In some embodiments, peptide segment comprise at least 50% sequence identity or similarity (e.g., conservative or semi-conservative) to one or more of the peptide sequences described herein.

In some embodiments, peptide amphiphiles comprise a charged peptide segment. The charged segment may be acidic, basic, or zwitterionic.

In some embodiments, peptide amphiphiles comprise an acidic peptide segment. For example, in some embodiments, the acidic peptide comprises one or more (e.g., 1, 2, 3, 4, 5, 6, 7, or more) acidic residues (D and/or E) in sequence. In some embodiments, the acidic peptide segment comprises up to 7 residues in length and comprises at least 50% acidic residues. In some embodiments, an acidic peptide segment comprises (Xa)₁₋₇, wherein each Xa is independently D or E. In some embodiments, an acidic peptide segment comprises E₂₋₄. For example, in some embodiments an acidic peptide segment comprises EE. In some embodiments, an acidic peptide segment comprises EEE. In other embodiments, an acidic peptide segment comprises EEEE (SEQ ID NO: 4).

In some embodiments, peptide amphiphiles comprise a basic peptide segment. For example, in some embodiments, the acidic peptide comprises one or more (e.g., 1, 2, 3, 4, 5, 6, 7, or more) basic residues (R, H, and/or K) in sequence. In some embodiments, the basic peptide segment comprises up to 7 residues in length and comprises at least 50% basic residues. In some embodiments, an acidic peptide segment comprises (Xb)₁₋₇, wherein each Xb is independently R, H, and/or K. For example, in some embodiments the basic peptide segment comprises KK, KKK, or KKKK (SEQ ID NO: 12).

In some embodiments, peptide amphiphiles comprises a structural peptide segment. In some embodiments, the structural peptide segment is a beta-sheet-forming segment. In some embodiments, the structural peptide segment displays weak hydrogen bonding and has the propensity to form random coil structures rather than rigid beta-sheet conformations. In some embodiments, the structural peptide segment is rich in one or more of H, I, L, F, V, G, and A residues. In some embodiments, the structural peptide segment comprises an alanine- and valine-rich peptide segment (e.g., VVAA (SEQ ID NO: 2), VVVAAA (SEQ ID NO: 3), AAVV (SEQ ID NO: 5), AAAVVV (SEQ ID NO: 6), VVAAA (SEQ ID NO: 7), VVVAA (SEQ ID NO: 8), or other combinations of V and A residues, etc.). In some embodiments, the structural peptide segment comprises 4 or more consecutive A and/or V residues, or conservative or semi-conservative substitutions thereto. In some embodiments, the structural peptide segment comprises V₂A₂(SEQ ID NO: 2).

In some embodiments, the structural peptide segment comprises an alanine and glycine-rich peptide segment (e.g. AAGG (SEQ ID NO: 16), AAAGGG (SEQ ID NO: 17), or other combinations of A and G residues, etc.). In some embodiments, the structural peptide segment comprises A₂G₂. In some embodiments, the structural peptide segment comprises GGGG (SEQ ID NO: 13).

In some embodiments, the structural peptide segment comprises 4 or more consecutive non-polar aliphatic residues (e.g., alanine (A), valine (V), leucine (L), isoleucine (I), methionine (M)). In some embodiments, the structural peptide segment comprises 2-16 amino acids in length and comprises 4 or more (e.g., 2, 3, 4, 5, 6, 7, 8, 9, 10, 11, 12, 13, 14, 15, 16, or ranges there between) non-polar aliphatic residues. In some embodiments, the structural peptide segment comprises VEVA₂ (SEQ ID NO: 18)

In some embodiments, the structural peptide segment has a total propensity for forming β-sheet conformations of 4 or less (e.g. less than 4, less than 3.9, less than 3.8, less than 3.7, less than 3.6, less than 3.5, less than 3.4, less than 3.3, less than 3.2, less than 3.1, less than 3.0, less than 2.9, less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, or less than 1.)

The total propensity for forming β-sheet conformations may be calculated as the sum of the propensity for forming β-sheet conformations of each amino acid in the structural peptide segment. The propensity of each amino acid for forming β-sheet conformations and methods for calculating the same are described in, for example, Fujiwara, K., Toda, H. & Ikeguchi, M. Dependence of α-helical and β-sheet amino acid propensities on the overall protein fold type. BMC Struct Biol 12, 18 (2012), the entire contents of which are incorporated herein by reference. Exemplary values are shown in Table 1, below. For the purposes of calculating the total propensity for forming β-sheet conformations of the structural peptide segment, the value shown in the “total residues” column from table 1 for each amino acid is added together. For example, for an A₂G₂ structural peptide segment, the total propensity for forming β-sheet conformations is 0.75+0.75+0.67+0.67=2.84. The structural peptide segment may comprise any suitable number and combination of amino acids to achieve a total propensity for forming β-sheet conformations of 4 or less.

TABLE 1
Amino acid Propensities for β-sheet conformations

| Amino Acid | Exposed Residues | Buried Residues | Total Residues |
|---|---|---|---|
| V | 2.31 | 1.57 | 2.00 |
| I | 2.02 | 1.39 | 1.79 |
| L | 1.18 | 0.93 | 1.15 |
| M | 1.01 | 0.84 | 1.01 |
| P | 0.49 | 0.42 | 0.40 |
| A | 0.48 | 0.72 | 0.75 |
| C | 1.24 | 1.07 | 1.36 |
| F | 1.50 | 1.10 | 1.4 |
| Y | 1.71 | 1.12 | 1.37 |
| W | 1.90 | 0.91 | 1.23 |
| Q | 0.96 | 0.82 | 0.72 |
| S | 0.86 | 0.85 | .081 |
| T | 1.58 | 1.08 | 1.21 |
| N | 0.71 | 0.76 | 0.63 |
| H | 1.15 | 0.98 | 0.99 |
| D | 0.61 | 0.76 | 0.55 |
| K | 1.14 | 0.98 | 0.76 |
| E | 0.89 | 0.86 | 0.65 |
| R | 1.27 | 0.82 | 0.85 |
| G | 0/41 | 0.81 | 0.67 |

In some embodiments, a structural peptide segment having a total propensity for forming β-sheet conformations of 4 or less indicates that the amino acids within the structural peptide segment have weaker interactions with neighboring molecules. For example, the structural peptide segment may display weak hydrogen-bonding abilities. Accordingly, such structural peptide segments and the peptide amphiphiles comprising the same may create more dynamic nanofiber structures. For example, an A₂G₂(SEQ ID NO: 16) structural peptide segment may display random coil structures rather than rigid beta-sheet conformations.

In some embodiments, peptide amphiphiles comprise a non-peptide spacer or linker segment. In some embodiments, the non-peptide spacer or linker segment is located at the opposite terminus of the peptide from the hydrophobic segment. In some embodiments, the spacer or linker segment provides the attachment site for a bioactive group (e.g. a netrin-1 mimetic sequence). In some embodiments, the spacer or linker segment provides a reactive group (e.g., alkene, alkyne, azide, thiol, maleimide etc.) for functionalization of the PA. In some embodiments, the spacer or linker is a substantially linear chain of CH₂, O, (CH₂)₂O, O(CH₂)₂, NH, and C═O groups (e.g., CH2(O(CH₂)₂)₂NH, CH2(O(CH₂)₂)₂NHCO(CH₂)₂CCH, etc.). In some embodiments, a spacer or linker further comprises additional bioactive groups, substituents, branches, etc. In some embodiments, the linker segment is a single glycine (G) residue. In some embodiments, the linker comprises GG. In some embodiments, the linker comprises GGG.

Suitable peptide amphiphiles for use in the materials herein, as well as methods of preparation of PAs and related materials, amino acid sequences for use in PAs, and materials that find use with PAs, are described in the following patents and applications: U.S. Pat. Nos. 9,044,514; 9,040,626; 9,011,914; 8,772,228; 8,748,569 8,580,923; 8,546,338; 8,512,693; 8,450,271; 8,236,800; 8,138,140; 8,124,583; 8,114,835; 8,114,834; 8,080,262; 8,076,295; 8,063,014; 7,851,445; 7,838,491; 7,745,708; 7,683,025; 7,554,021; 7,544,661; 7,534,761; 7,491,690; 7,452,679; 7,371,719; 7,030,167; and WO2020154631A1; all of which are herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural peptide segment, bioactive segment, etc.). For example, nanofibers, nanospheres, intermediate shapes, and other supramolecular structures are achieved by adjusting the identity of the PA component parts. In some embodiments, characteristics of supramolecular nanostructures of PAs are altered by post-assembly manipulation (e.g., heating/cooling, stretching, etc.).

In some embodiments, a peptide amphiphile comprises: (a) a hydrophobic tail comprising an alkyl chain of 8-24 carbons; (b) a structural peptide segment (e.g., comprising VVAA (SEQ ID NO: 2); and (c) a charged segment (e.g., comprising EE, EEE, EEEE (SEQ ID NO: 4), etc.). In some embodiments, any PAs within the scope described herein, comprising the components described herein, or within the skill of one in the field, may find use herein.

In some embodiments, peptide amphiphiles comprise a netrin-1 mimetic sequence. A netrin-1 mimetic sequence is considered a “bioactive moiety”. In particular embodiments, the netrin-1 mimetic sequence is the most C-terminal or N-terminal segment of the PA. In some embodiments, the netrin-1 mimetic sequence is attached to the end of the charged segment. In some embodiments, the netrin-1 mimetic sequence is exposed on the surface of an assembled PA structure (e.g., nanofiber).

In some embodiments, the netrin-1 mimetic sequence comprises IDP. In some embodiments, the netrin-1 mimetic sequence comprises IDP blanked by two cysteine residues (e.g. CIDPC) (SEQ ID NO: 1). In some embodiments, the netrin-1 mimetic sequence comprises CIDPC, and the flanking cysteine residues induce cyclization of the netrin-1 mimetic sequence. In some embodiments, cyclization of the netrin-1 mimetic sequence improves properties of the netrin-1 mimetic peptide amphiphile compared to a peptide amphiphile comprising a linear netrin-1 mimetic sequence, such as SIDPS (SEQ ID NO: 9).

In some embodiments, the netrin-1 mimetic sequence is cyclized. Use of two flanking cysteine residues, as above, is an exemplary method for cyclization of the netrin-1 mimetic peptide. Use of flanking cysteine residues promotes cyclization by formation of a disulfide bond (i.e. disulfide bridge) between the cysteine residues. Alternative methods for inducing cyclization include methods that promote the formation of lactam bridges, lactone and thiolactone bridges, thioether and ether bridges, and the like. For example, the netrin-1 mimetic sequence (e.g. IDP) can be flanked by suitable residues to form the desired bridge, thus inducing cyclization of the peptide. Any suitable strategy for forming a cyclic netrin-1 mimetic peptide can be used, including formation of lactam bridges (e.g. between flanking glutamic/aspartic acid and lysine residues), formation of lactone and thiolactone bridges (e.g. between flanking amino acids containing carboxyl, hydroxyl, or mercapto functional groups), and formation of thioether or ether bridges (e.g. between flanking amino acids containing hydroxyl or mercapto functional groups).

In some embodiments, a peptide amphiphile comprises (e.g., from C-terminus to N-terminus or from N-terminus to C-terminus): netrin-1 mimetic sequence (e.g. CIDPC (SEQ ID NO: 1))—charged segment (e.g., comprising EE, EEE, EEEE (SEQ ID NO: 4), etc.)—structural peptide segment (e.g., comprising V₂A₂(SEQ ID NO: 2)—hydrophobic tail (e.g., comprising an alkyl chain of 8-24 carbons).

In some embodiments, a peptide amphiphile comprises (e.g., from C-terminus to N-terminus or from N-terminus to C-terminus): netrin-1 mimetic sequence (e.g. CIDPC (SEQ ID NO: 1))—flexible linker (e.g. GG)—charged segment (e.g., comprising EE, EEE, EEEE (SEQ ID NO: 4), etc.)—structural peptide segment (e.g., comprising V₂A₂(SEQ ID NO: 2)-hydrophobic tail (e.g., comprising an alkyl chain of 8-24 carbons).

In some embodiments, a PA further comprises an attachment segment or residue (e.g., K) for attachment of the hydrophobic tail to the peptide portion of the PA. In some embodiments, the hydrophobic tail is attached to a lysine side chain.

In some embodiments, provided herein are nanofibers and nanostructures assembled from the peptide amphiphiles described herein. In some embodiments, a nanofiber is prepared by the self-assembly of the PAs described herein. In some embodiments, a nanofiber comprises or consists of netrin-1 mimetic PAs (e.g. PAS comprising the netrin-1 mimetic sequence). In some embodiments, the netrin-1 mimetic sequences are displayed on the surface of the nanofiber. In some embodiments, in addition to netrin-1 mimetic PAs, filler PAs are included in the nanofibers. In some embodiments, filler PAs are peptide amphiphiles, as described herein (e.g., structural peptide segment, charged segment, hydrophobic segment, etc.), but lacking a bioactive segment (e.g. lacking a netrin-1 mimetic sequence). In some embodiments, filler peptides are basic or acidic peptides lacking a bioactive segment. In some embodiments, the filler PAs and netrin-1 mimetic PAs self-assemble into a nanofiber comprising both types of PAs. In some embodiments, nanostructures (e.g., nanofibers) assembled from the peptide amphiphiles described herein are provided.

In some embodiments, filler peptides (e.g., basic peptide, acidic peptides, etc.) impart mechanical characteristics to a material comprising the PA nanofibers described herein. In some embodiments, a nanofiber assembled from 0-75% (mass %) netrin-1 mimetic PA and 25-100% (mass %) basic filler PA becomes a gel at basic pH conditions (e.g., pH 8.5-11). In some embodiments, a nanofiber assembled from 75-100% (mass %) netrin-1 mimetic PA and 0-25% (mass %) basic filler PA is a liquid at basic pH conditions (e.g., pH 8.5-11). In some embodiments, a nanofiber assembled from 0-20% (mass %) netrin-1 mimetic PA and 80-100% (mass %) acidic filler PA becomes a gel at acidic pH conditions (e.g., pH 1-5). In some embodiments, a nanofiber assembled from 20-80% (mass %) netrin-1 mimetic PA and 20-80% (mass %) acidic filler PA becomes a gel at neutral pH conditions (e.g., pH 5-8.5). In some embodiments, a nanofiber assembled from 80-100% (mass %) netrin-1 mimetic PA and 0-20% (mass %) acidic filler PA is a liquid at acidic pH conditions (e.g., pH 1-5).

In some embodiments, nanostructures are assembled from (1) netrin-1 mimetic PAs and (2) filler PAs (e.g., acidic or basic PAs not-labeled or not displaying a netrin-1 mimetic sequence) In some embodiments, nanostructures (e.g., nanofibers) comprise about 10% to about 40% netrin-1 mimetic peptide amphiphiles and about 60% to about 90% filler peptide amphiphiles. In some embodiments, nanostructures (e.g., nanofibers) comprise about 10% to about 40% netrin-1 mimetic peptide amphiphiles, about 10% to about 38% netrin-1 mimetic PAs, about 10% to about 36% netrin-1 mimetic PAs, about 10% to about 34% netrin-1 mimetic PAs, about 10% to about 32% netrin-1 mimetic PAs, about 10% to about 30% netrin-1 mimetic PAs, or about 15% to about 30% netrin-1 mimetic PAs. In some embodiments, the nanostructures (e.g. nanofibers) comprise about 60% to about 90% filler PAs, about 62% to about 90% filler PAs, 64% to about 90% filler PAs, about 66% to about 90% filler PAs, about 68% to about 90% filler PAs, about 70% to about 90% filler PAs, or about 70% to about 85% filler PAs. In some embodiments, the ratio of netrin-1 mimetic PA to acidic and/or basic PAs in a nanofiber determines the mechanical characteristics (e.g., liquid or gel) of the nanofiber material and under what conditions the material will adopt various characteristics (e.g., gelling upon exposure to physiologic conditions, liquifying upon exposure to physiologic conditions, etc.).

Peptide amphiphile (PA) nanofiber solutions may comprise any suitable combination of PAs. In some embodiments, at least 0.05 mg/mL (e.g., 0.10 mg/ml, 0.15 mg/ml, 0.20 mg/ml, 0.25 mg/ml, 0.30 mg/ml, 0.35 mg/ml, 0.40 mg/ml, 0.45 mg/ml, 0.50 mg/ml, 0.60 mg/ml, 0.70 mg/ml, 0.80 mg/ml, 0.90 mg/ml, 1.0 mg/ml, or more, or ranges therebetween), of the solution is a filler PA (e.g., without a bioactive segment). In some embodiments, at least 0.25 mg/mL of the solution is a filler PA. In some embodiments, a filler PA is a non-bioactive PA molecule having highly charged glutamic acid residues on the terminal end of the molecule (e.g., surface-displayed end). These negatively charged PAs allow for the gelation to take place between nanofibers via ionic crosslinks. In some embodiments, a filler PA is a non-bioactive PA molecule having highly charged lysine residues on the terminal end of the molecule (e.g., surface-displayed end). These positively charged PAs allow for the gelation to take place under basic conditions. The filler PAs provide the ability to incorporate other bio-active PAs molecules into the nanofiber matrix while still ensuring the ability of the nanofibers solution to gel. In some embodiments, the solutions are annealed for increased viscosity and stronger gel mechanics. These filler PAs have sequences are described in, for example, U.S. Pat. No. 8,772,228 (e.g., C₁₆-V₂A₂E₂) (SEQ ID NO: 10) which is herein incorporated by reference in its entirety.

In some embodiments, the PA nanofiber described herein exhibit a small cross-sectional diameter (e.g., <25 nm, <20 nm, <15 nm, about 10 nm, etc.). In some embodiments, the small cross-section of the nanofibers (˜10 nm diameter) allows the fibers to permeate the brain parenchyma.

In some embodiments, the PAs, systems, and nanofibers described herein may be incorporated into pharmaceutical compositions for use in methods of treating a disease, disorder, condition, or injury in a subject. In some embodiments, the PAs, systems, and nanofibers described herein are incorporated herein into a pharmaceutical composition for promoting neuronal growth, maturation, and/or signaling in a subject. In some embodiments, the subject has a disease or condition that causes neuronal injury and/or death, and the compositions provided herein improve one or more symptoms of the disease or condition by promoting neuronal growth, maturation, and/or signaling in the subject. For example, the PAs and nanofibers described herein may be used for methods of treatment or prevention of nervous system injury in a subject. For example, the PAs and nanofibers described herein may be used in methods for treatment of prevention of injury to the central nervous system (CNS), including the brain and the spinal cord, or the peripheral nervous system (PNS), including the nerves and ganglia outside of the brain and spinal cord. In some embodiments, the PAs and nanofibers described herein may be used for treatment or prevention of injury to the CNS or PNS in a subject. In some embodiments, the injury is a spinal cord injury. The spinal cord injury may be cervical, lumbar, thoracic, sacral, or any combination thereof. In some embodiments, the injury is a brain injury.

The injury may be a traumatic injury. A traumatic injury refers to an injury caused by trauma, for example trauma such as that caused by an automobile accident, a fall, violence, sports injury, surgical injury, and the like.) For example, the PAs and nanofibers described herein may be used for the treatment of traumatic central nervous system injury (e.g. traumatic spinal cord injury, traumatic brain injury (TBI). Alternatively, the injury may be a non-traumatic injury. For example, the injury may be a non-traumatic injury to the CNS (e.g., the brain and/or the spinal cord) or the PNS caused by, for example, cancer, multiple sclerosis, inflammation, arthritis, spinal stenosis, tumors, blood loss, stroke, and the like. In some embodiments, the compositions are used as therapy for diseases known affected by a lack of netrin-1 upregulation, including Alzheimer's disease, Parkinson's disease, and stroke.

In some embodiments, administration of a composition comprising a PA or nanofiber described herein increases neurite outgrowth, reduces inflammation, reduces glial scar formation, and/or improves functional recovery following a central nervous system injury (e.g. a spinal cord injury, a brain injury) in a subject. “Treating” a central nervous system injury is intended to encompass any one of more of the above outcomes (e.g. any one of more of increasing neurite outgrowth, reducing inflammation, reducing glial scar formation, and improving functional recovery following the injury). Improving functional recovery may refer to improving movements in the legs after a spinal cord injury, e.g. improving walking, balance, flexibility, etc.

A composition comprising PAs and/or nanofibers described herein may be provided to a subject at any suitable point following injury (e.g. CNS injury) to treat the injury. For example, the composition may be provided to the subject within 24 hours of the injury (e.g. within 24 hours, within 12 hours, within 10 hours, within 9 hours, within 8 hours, within 7 hours, within 6 hours, within 5 hours, within 4 hours, within 3 hours, within 2 hours, or within 1 hour from injury. In some embodiments, the composition may be provided to the subject after a duration longer than 24 hours has passed following injury or diagnosis of injury.

The composition may be administered in any suitable amount, depending on factors including the age of the subject, weight of the subject, severity of the injury, and the like. The composition may be administered in combination with other suitable treatments for injury or preventative measures to prevent the severity of the injury from worsening.

In some embodiments, the PA and nanofiber compositions herein are formulated for delivery to a subject. In some embodiments, the compositions are formulated for parenteral administration (e.g. by injection). Suitable routes of administration include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the PA compositions are administered parenterally. In some embodiments, parenteral administration is by intrathecal administration, intracerebroventricular administration, or intraparenchymal administration. The PA compositions herein can be administered as the sole active agent or in combination with other pharmaceutical agents such as other agents used in the treatment of nervous system injury in a subject.

In some embodiments, the PAs and nanofibers described herein find use in cell/organoid culture methods. For example, further disclosed herein are scaffolds (e.g. hydrogels) comprising the peptide amphiphiles described herein. The scaffolds may comprise a nanofiber of self-assembled peptide amphiphiles, at least a portion of the peptide amphiphiles comprising: a hydrophobic tail, a structural peptide segment, a charged peptide segment, and a netrin-1 mimetic sequence. The scaffold may further comprise one or more filler peptide amphiphiles. The scaffolds described herein are capable of supporting growth and differentiation of a cell. Accordingly, the scaffolds may be in methods for culturing cells or organoids. The methods for culturing cells or organoids comprise contacting the cells or organoids with a scaffold as described here. In some embodiments, the scaffold may be used as a coating for any desired cell culture tool (tissue culture plate, petri dish, glass slide, etc.).

Cells or organoids cultured on the scaffolds disclosed herein may demonstrate improved characteristics compared to cells or organoids cultured in the absence of the disclosed scaffolds. For example, cells or organoids may demonstrate improved differentiation, improved maturation and/or improved long term viability compared to cells or organoids cultured in the absence of the disclosed scaffolds. In some embodiments, the scaffolds may be used in methods of culturing neuronal cells (e.g. neurons). In some embodiments, neurons cultured on the scaffolds provided herein display enhanced synaptogenesis and functional maturation (e.g. neurite outgrowth, axonal projections, improved electrical activity (e.g. cell signaling) etc.) compared to neurons cultured on a scaffold lacking the netrin-1 mimetic PA. In some embodiments, the scaffolds may be used in methods of culturing neural organoids, neurospheroids, and the like.

### EXPERIMENTAL

### Example 1

Netrin-1, a chemotropic factor present throughout the nervous system, initiates axon guidance, outgrowth, and branching, as well as synaptogenesis, through the activation of deleted in colorectal cancer (DCC) receptors. However, there are concerns about the stability and overall efficacy of netrin-1 in vivo. Herein, it was evaluated whether netrin-1 mimetic nanostructures offer potential regenerative functions for the CNS. To accomplish this, IDP netrin-1 mimetic peptide-functionalized supramolecular nanostructures were developed and their effect on neuron growth and maturation was assessed. Efficacy in promoting recovery in a mouse model of spinal cord injury was also assessed.

The results presented herein provide a nanofiber-shaped supramolecular mimetic of netrin-1 with monomers that incorporate a cyclic peptide sequence (CIDPC, SEQ ID NO: 1) as the bioactive component. The mimetic structure referred to as Netrin-1 PA was found to activate the DCC receptor in primary cortical neurons using low molar ratios of the bioactive comonomer. The supramolecular nanofibers enhanced neurite outgrowth and upregulated maturation as well as pre- and postsynaptic markers over time, resulting in differences in electrical activity similar to neurons treated with the recombinant netrin-1 protein. The results suggest the possibility of using the supramolecular structure as a therapeutic to promote regenerative bioactivity in CNS injuries. Moreover, functional recovery after spinal cord injury was increased in mise treated with the Netrin-1 PA compared to Pas lacking the Netrin-1 mimetic sequence (E2 PA) and sham, accompanied by reduction in glial scar formation and reduced inflammation at the site of injury. Taken together, these results establish the efficacy of Netrin-1 PA in treating CNS injury (e.g. spinal cord injury) and promoting neural regeneration.

### Results

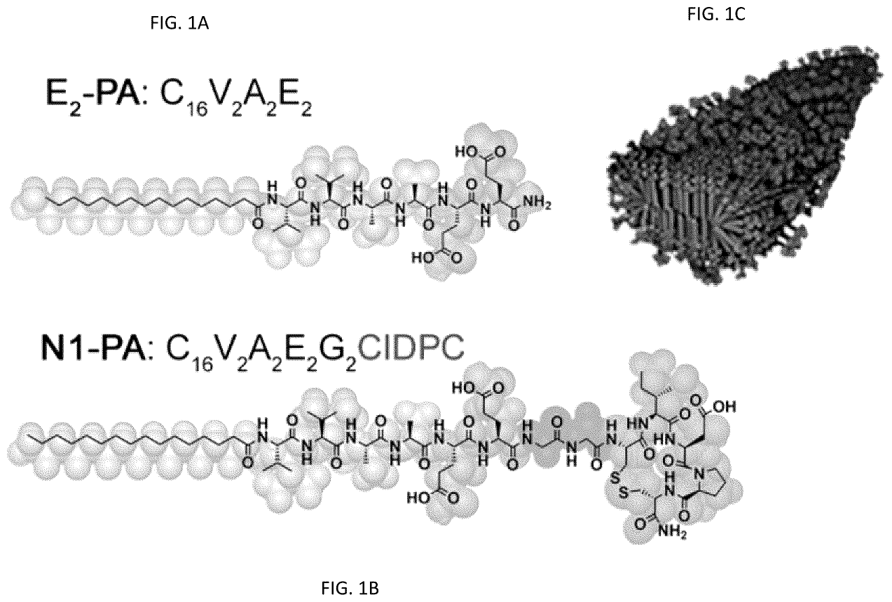

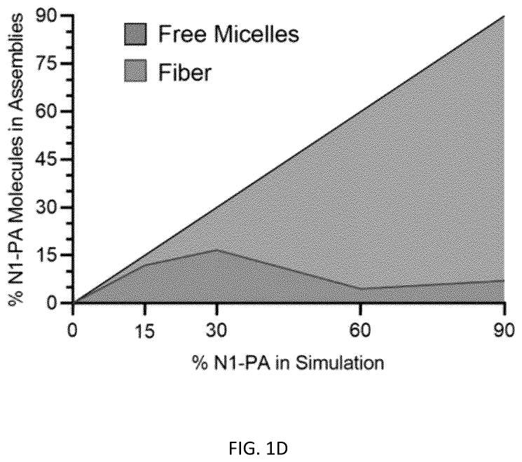

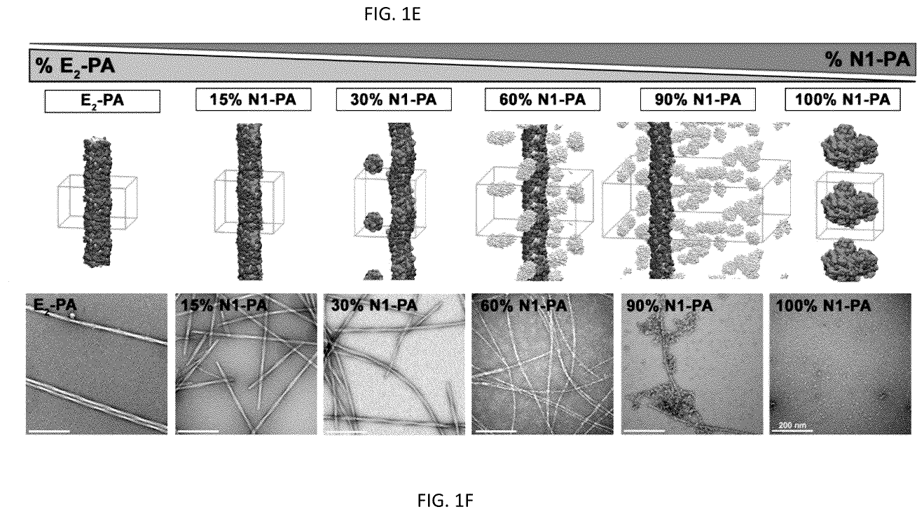

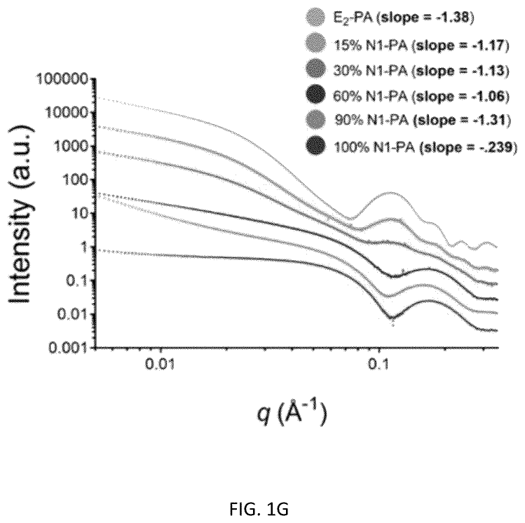

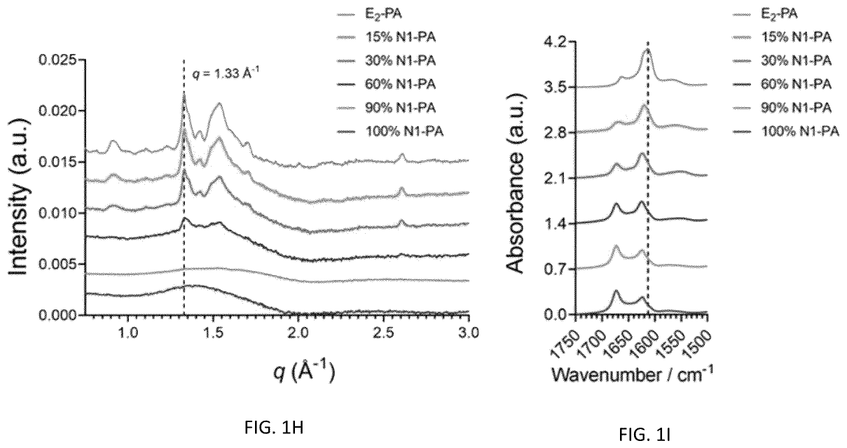

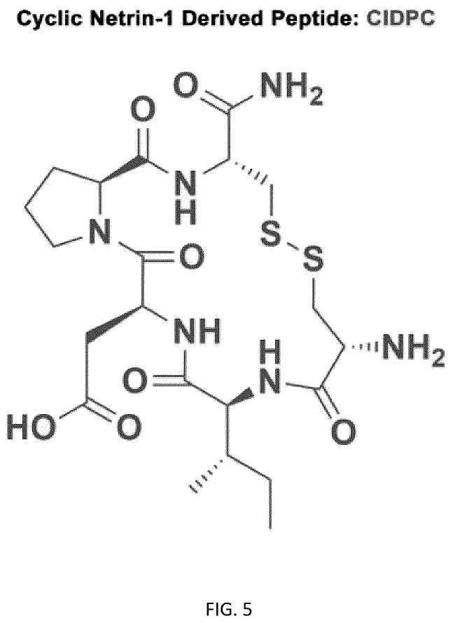

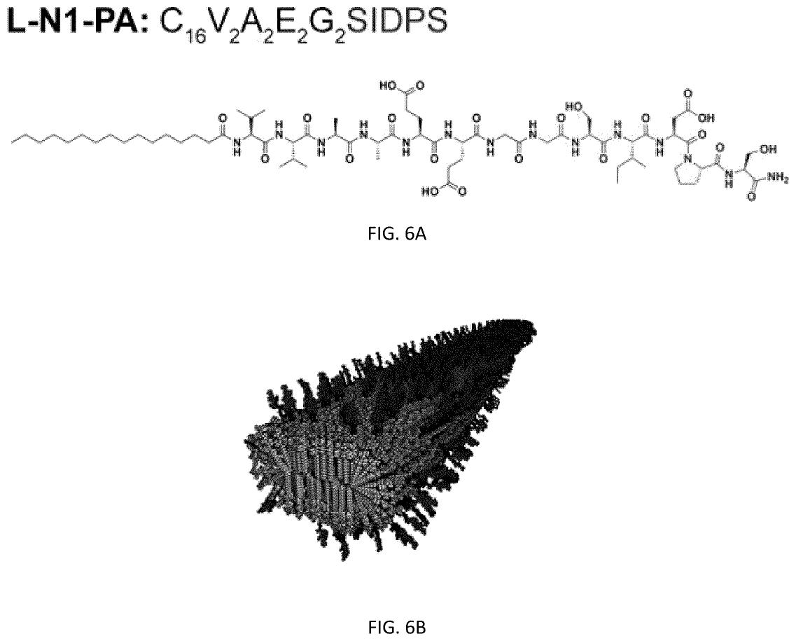

PA Design and Characterization. The PA molecule C₁₆-V₂A₂E₂ (SEQ ID NO: 10) (E₂-PA) (FIG. 1A) was used as a “backbone” due its ability to assemble into high-aspect-ratio nanoribbons in solution. E₂-PA also forms robust hydrogels under physiological conditions, with a storage modulus of approximately 4 kPa, similar to that of native neural tissue. The N1-PA (FIG. 1B) was designed by adding two glycine residues that link the netrin-1 mimetic cyclic peptide sequence, CIDPC (SEQ ID NO: 1) (FIG. 5), to the C-terminus of the E₂-PA molecule. The inclusion of two glycine residues was intended to enhance access of the bioactive sequence to cell receptors. In addition, a linear N1-PA (L-N1-PA) molecule was synthesized as a control to probe how the more constrained conformational space of looplike bioactive domains affects their interaction with cell receptors (FIG. 6A). The L-N1-PA design still utilized the IDP bioactive peptide sequence but replaced the flanking cysteines with serine residues (cysteines were used for cyclization through disulfide bonding), and thus had the sequence SIDPS (SEQ ID NO: 9). To create supramolecular structures displaying the bioactive peptides at various densities, the cyclic and linear bioactive PA molecules were mixed and annealed with the nonbioactive E₂-PA (FIG. 1C, FIG. 6B).

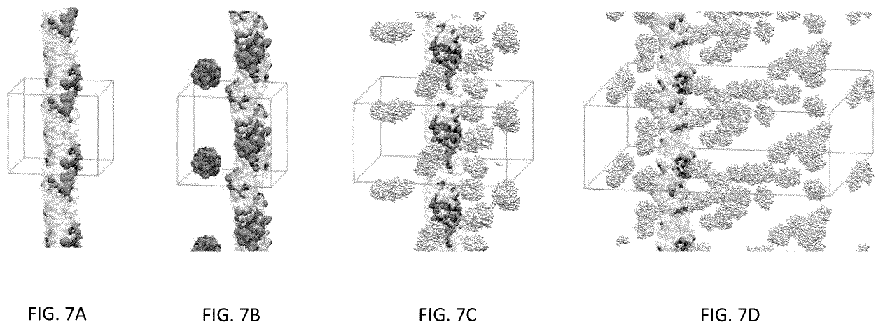

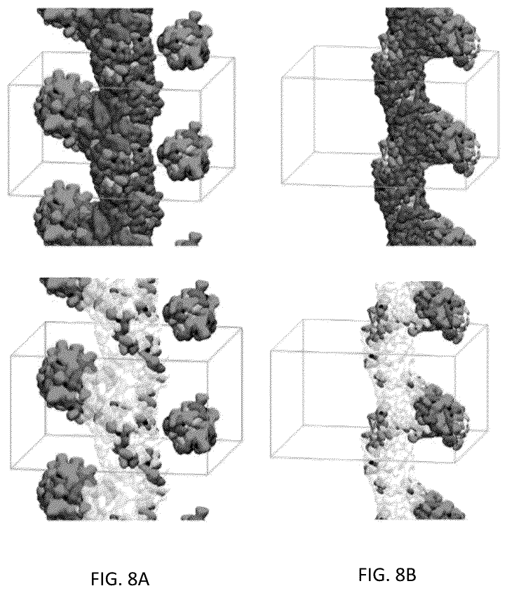

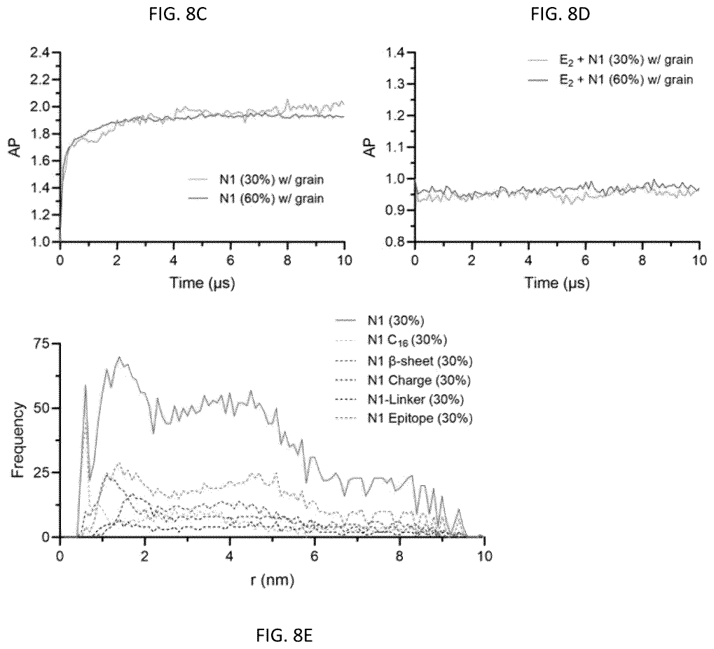

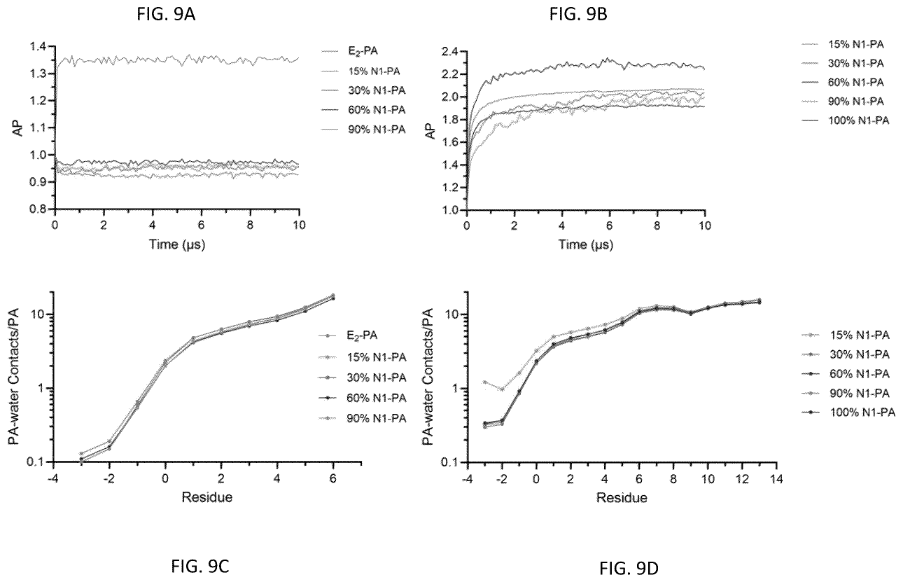

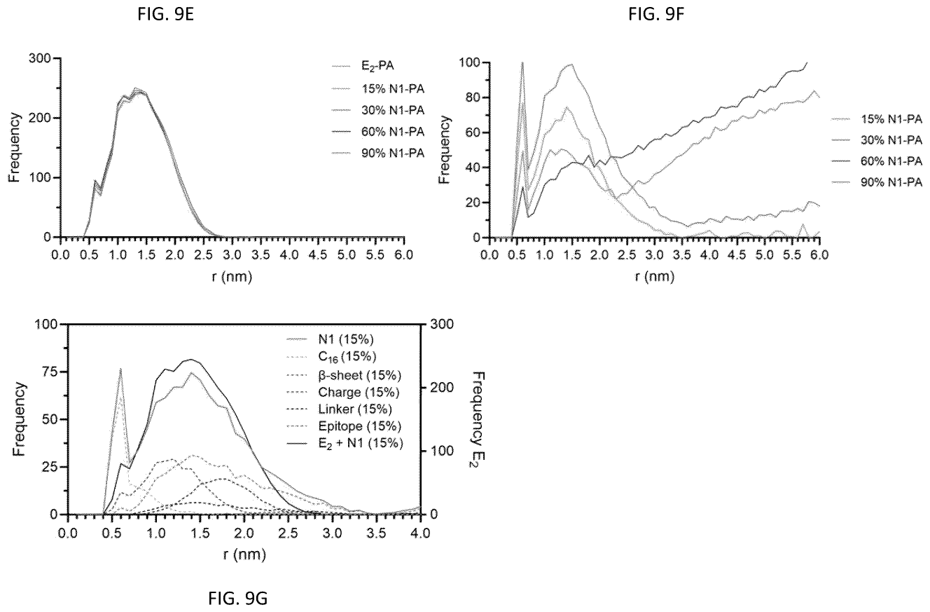

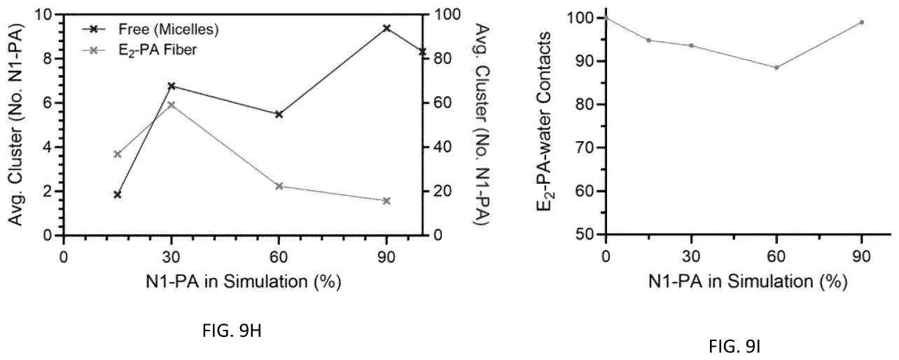

To evaluate the co-assembly of the N1-PA and E₂-PA into supramolecular nanofibers, coarse-grained molecular dynamics (CG-MD) simulations were performed using different molar ratios. In these simulations, variable amounts of N1-PA were added to an E₂-PA nanofiber consisting of 300 molecules to achieve molar ratios of 15, 30, 60, and 90%. Interestingly, simulations show that the integration of N1-PA into the E₂-PA assembly only increases from 11.9% to 16.7% when the N1-PA is doubled from 15% to 30% and drastically decreases to 4.5% and 7.1% when the N1-PA in the system is further increased to 60% and 90%, respectively (FIG. 1D). Simulation snapshots showed that at low molar ratios N1-PA is successfully incorporated into the E₂-PA assembly (FIGS. 1E and A and B). As the molar ratio of N1-PA is increased, the proportion of unassembled N1-PA increases, instead forming micellar aggregates (FIGS. 1E and 7C and D). At higher percentages, these micelles interacted strongly with the fibers and adhered to their surface without being properly incorporated into the supra-molecular nanofiber (FIGS. 8 and 9E-G). Furthermore, analysis of clusters suggests the existence of a critical N1-PA micelle size that strongly competes with the incorporation of N1-PA into the E₂-PA fibers (FIG. 9H). Therefore, based on the evaluation of the CG-MD simulations, there appears to be a counterproductive effect on the incorporation of N1-PA into the E₂-PA fibers above a 30% molar ratio of the bioactive cyclic monomer.

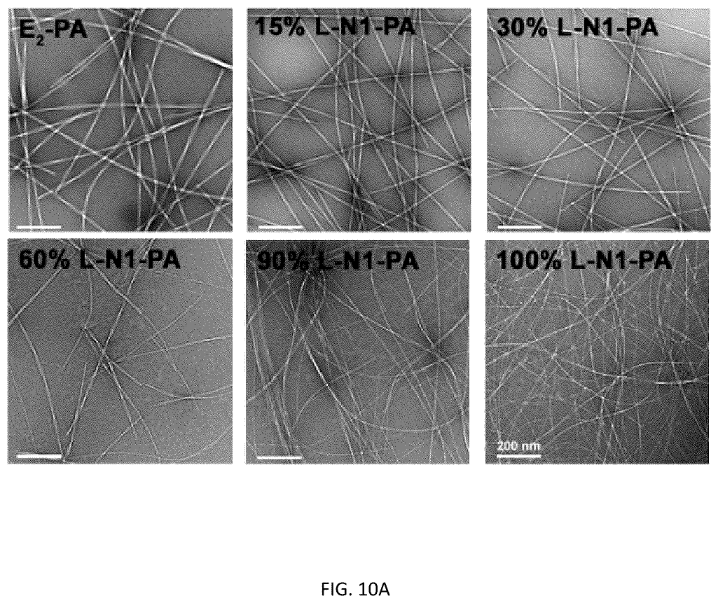

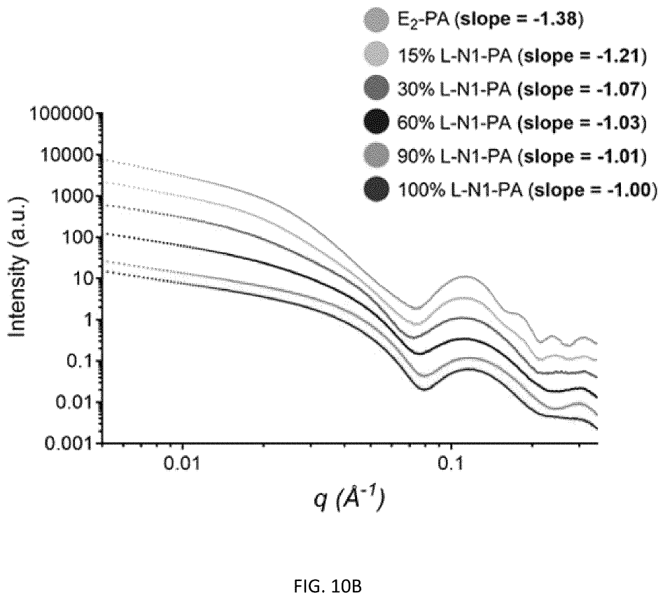

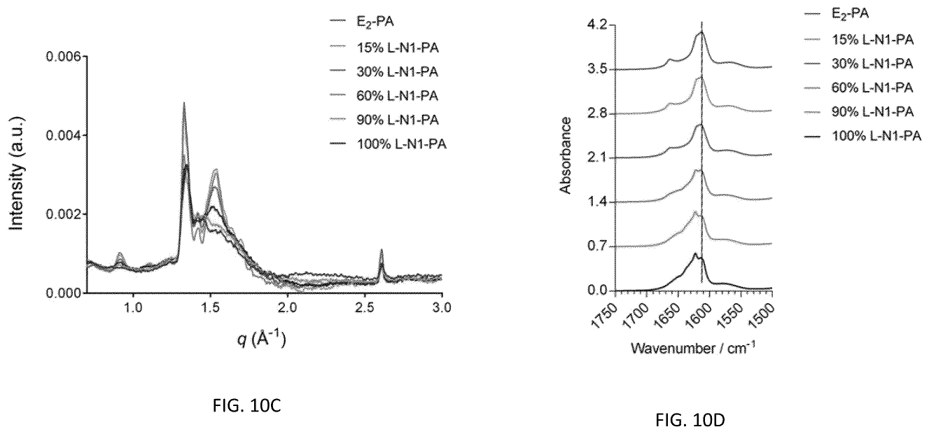

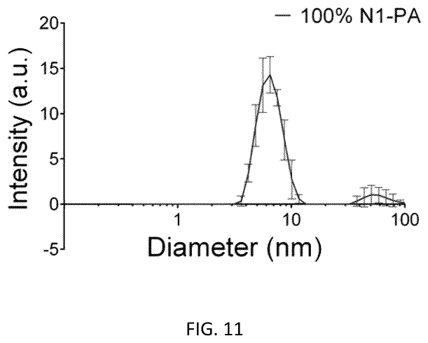

Next, transmission electron microscopy (TEM) (FIGS. 1F and 10A) and small-angle X-ray scattering (SAXS) measurements were used to characterize the morphologies of both the supramolecular nanostructures (FIGS. 1G and 10B). Using TEM, we found that E₂-PA alone and E₂-PA mixed with low molar ratios (15% and 30%) of N1-PA formed twisted ribbon-like structures. However, when E₂-PA was mixed with high molar ratios (60% and 90%) of N1-PA, the nanostructures acquired a cylindrical morphology of smaller diameter than E₂-PA alone, with visible aggregation into micelles that appeared to be dominant at 100% N1-PA. The experimental observations by TEM of micelle-decorated fibers at 60% and 90% N1-PA are in agreement with the simulations (FIGS. 1E and 8). Consistent with the CG-MD and TEM results, the SAXS scans revealed slopes of −1.1 to −1.4 in the Guinier region, confirming the existence of one-dimensional nano-structures of E₂-PA alone and the 15%, 30%, 60%, and 90% N1-PA assemblies. In addition, as the percentage of N1-PA in the assemblies increased, the minima present at 0.074 Å⁻¹ in the E₂-PA SAXS profile shifted to higher q values, corresponding to a decrease in the diameter of nanofibers. The 100% N1-PA had a slope of nearly 0, indicating a spherical micelle morphology. SAXS and TEM revealed the gradual transition from fibers to micelles as the molar ratio of N1-PA increased in the assemblies. The micellar structures were further characterized by dynamic light scattering (DLS), which confirmed the presence of a relatively uniform population of spherical micelles in 100% N1-PA samples (FIG. 11). In contrast, co-assemblies of L-N1-PA with molar ratios ranging from 15% to 100% retained their high-aspect-ratio morphology, as observed by TEM. SAXS profiles showed that as the molar ratio of L-N1-PA increased, the slope became less negative and approached −1.0, suggesting a transformation from ribbon-like structures to cylindrical fibers consistent with our observations by TEM (FIG. 10A).

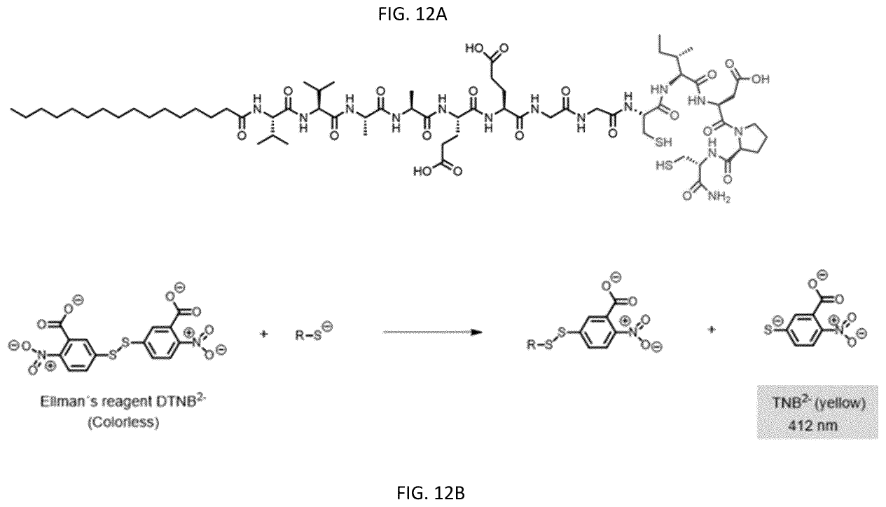

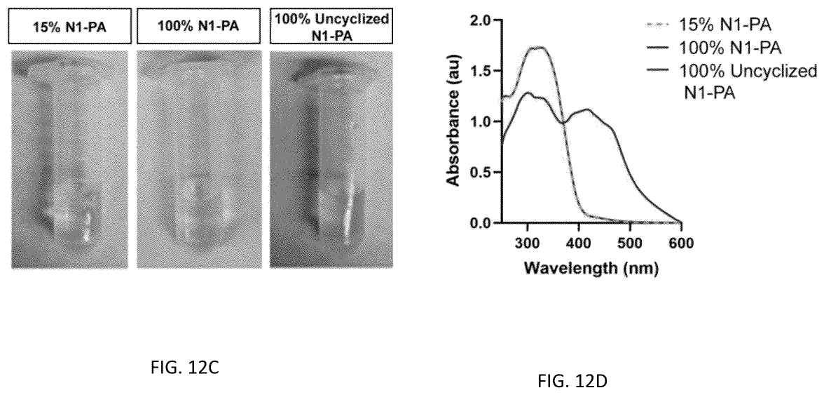

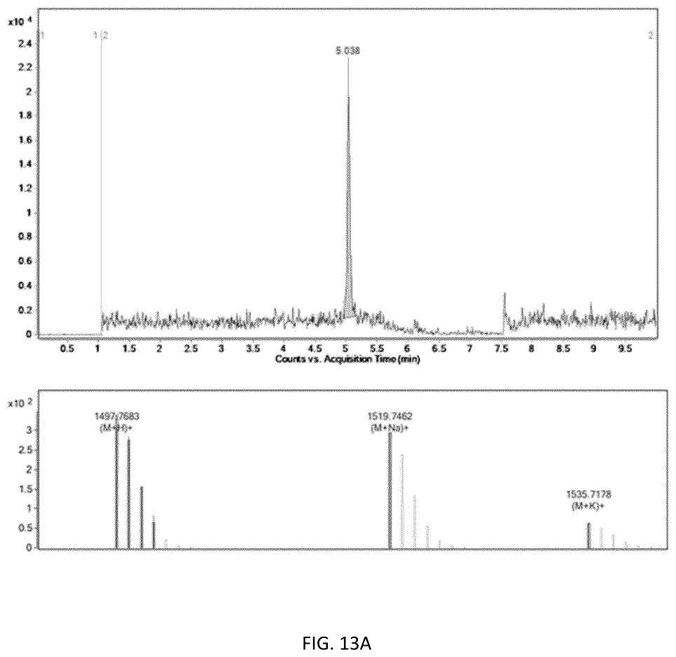

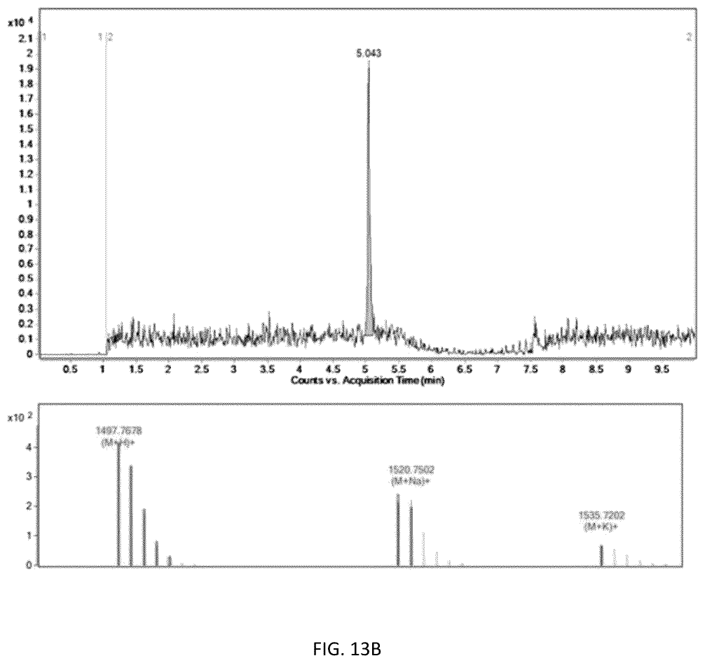

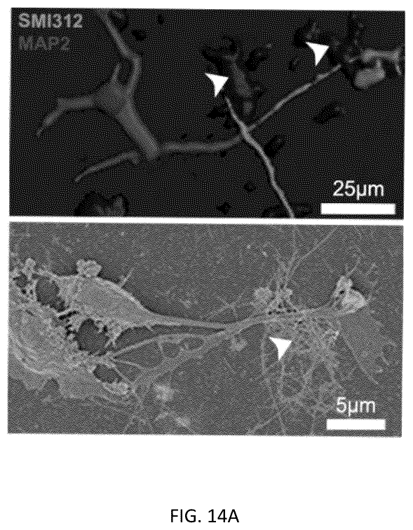

As evidence for the preservation of CIDPC cyclization in N1-PA monomers, Ellman's reagent, a molecule that undergoes a chemical reaction in the presence of free thiols to shift from colorless to yellow (412 nm), was added to solutions of 15% N1-PA, 100% N1-PA, and an uncyclized 100% N1-PA control containing free cysteines (FIGS. 12A and B). Solutions containing uncyclized N1-PA emitted a yellow color, and cyclized N1-PA remained colorless, based on both qualitative observation and absorbance measurements (FIGS. 12C and D). In addition, it was investigated whether the cyclized N1-PA would maintain its disulfide bonds in the presence of glutathione (GSH), a reducing agent present in cell culture. Using liquid chromatograph-mass spectroscopy (LC-MS), it was found that the cyclic N1-PA molecular structure was maintained 48 h after the addition of GSH in solution (FIGS. 13A and B), suggesting that the disulfide bond that cyclizes the CIDPC netrin-1 mimetic sequence would be maintained in vitro. These results confirm that the cyclic conformation of the N1-PA monomer is conserved within supramolecular assemblies and rationalizes further comparison to L-N1-PA.

Having identified the morphological variation at increasing molar ratios of the epitope-conjugated PAs by SAXS and TEM, the intermolecular hydrogen bonding interactions of N1-PA and L-N1-PA with diluent E₂-PA were investigated. These interactions among molecules within the nanofibers can reduce supramolecular motion and consequently reduce the interaction of signals with receptors. Both wide-angle X-ray scattering (WAXS) and transmission Fourier transform infrared spectroscopy (FTIR) measurements were performed in an effort to characterize the internal order of the molecules within N1-PA and L-N1-PA nanofibers. WAXS scans revealed that increasing the concentration of either N1-PA or L-N1-PA leads to a decrease in the peak intensity at q=1.33 Å⁻¹ corresponding to the intermolecular spacing of 4.72 Å, which is characteristic of f-sheets with their high density of hydrogen bonds. At N1-PA molar ratios of 90% and 100%, a β-sheet peak is not observed (FIG. 1H), whereas L-N1-PA assemblies at molar ratios of 15% to 100% retained their internal β-sheet structure with some variation in peak intensity (FIG. 10C). These observations using WAXS were further validated by FTIR (FIGS. 1I and 10D). As the ratio of N1-PA increased, the band in the amide I region present in 100% E₂-PA nanofibers at approximately 1613 cm⁻¹ gradually shifted toward higher wavenumbers approaching 1625 cm¹, indicating a less restricted hydrogen bonding network in the β-sheet structures (FIG. 1I). In the assemblies of 15% and 30% N1-PA, the nanostructures are predominantly fibers, and a significant population of spherical micelles was not observed by SAXS and TEM. Thus, the decreased internal order and weakened hydrogen bonding network revealed by WAXS and FTIR are likely caused by the incorporation of N1-PA molecules into the nanofibers, which is consistent with the molecular co-assembly predicted by CG-MD. Without wishing to be bound by theory, it was hypothesized that the weakened internal order induced by N1-PA monomers enhances intrafiber supramolecular motion, which should be beneficial to biological signaling of receptors. L-N1-PA assemblies that showed a similar shift of the amide-I peak, with the band present at 100% E₂-PA gradually shifting toward 1623 cm⁻¹, were also characterized by FTIR (FIG. 10D). In contrast to N1-PA, high concentrations of L-N1-PA within the co-assembly did not result in a significant reduction of the amide-I peak, indicating the preservation of the β-sheet structure, as suggested by WAXS experiments. Both WAXS and FTIR results suggest that N1-PA supramolecular nanostructures possess a less restricted hydrogen bonding network in the β-sheet-forming region, indicating the potential for an enhanced biological effect in comparison to L-N1-PA.

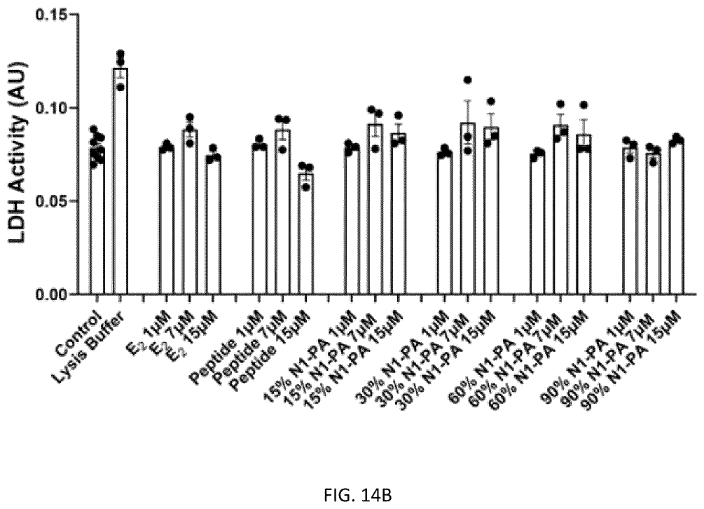

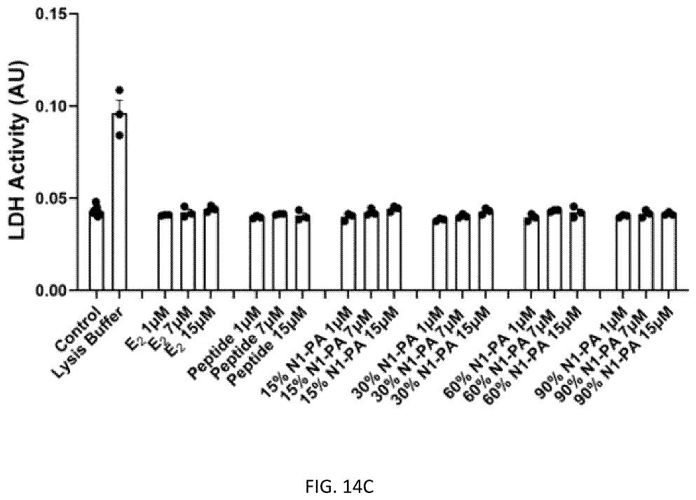

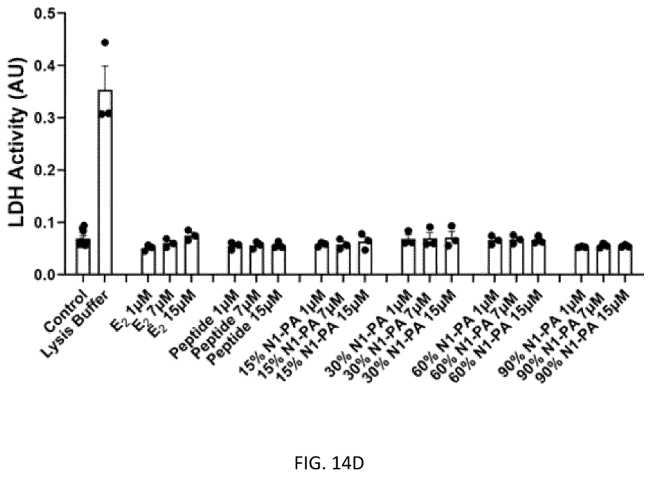

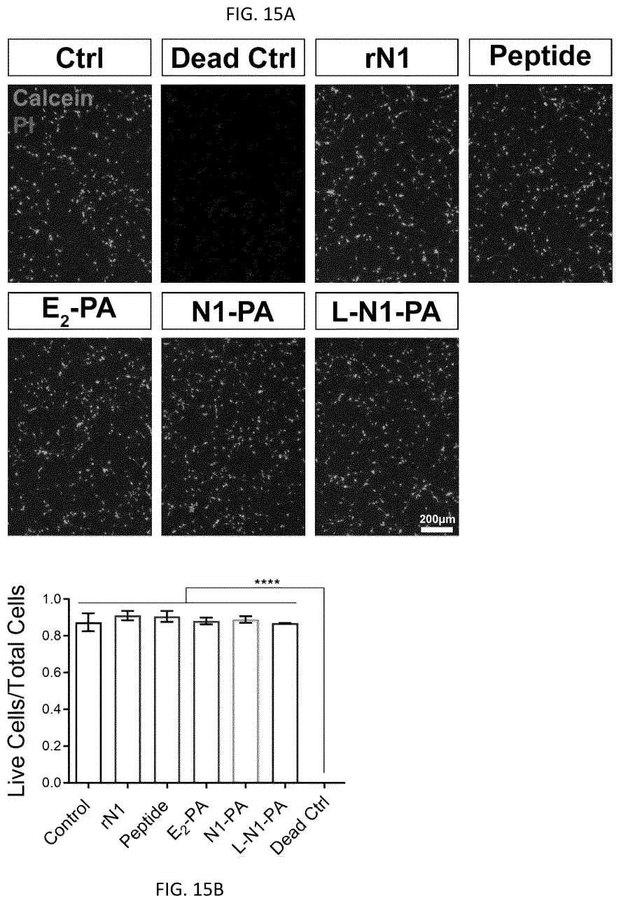

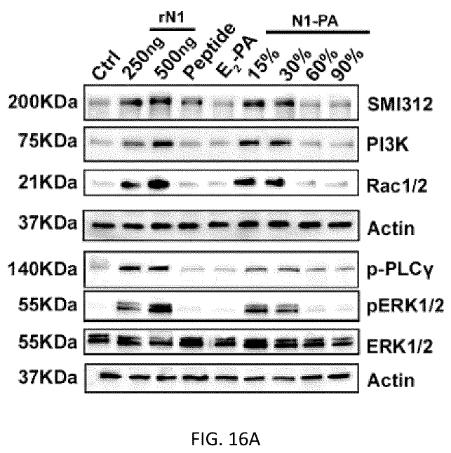

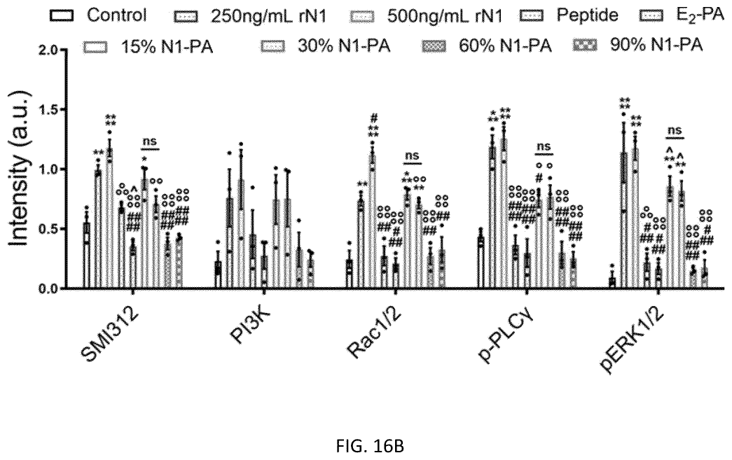

N1-PA Activates DCC Receptor-Related Pathways. The ability of N1-PA supramolecular nanofibers in solution to mimic the recombinant netrin-1 protein (rN1) was next investigated using primary mouse cortical neurons in vitro. Fiber-forming N1-PA assemblies were assessed in a concentration sweep consisting of 1, 7, and 15 μM. Confocal microscopy and scanning electron microscopy (SEM) revealed the presence of PA nanofibers in contact with neurons 24 h after initial treatment (see material indicated by white arrows in FIG. 14A). While some minor aggregation of PA was observed, the surfaces of individual nanofibers were still visible, suggesting that the netrin-1 mimetic peptide was still accessible to cell receptors. Neuron cultures treated with N1-PA co-assemblies at 1, 7, and 15 μM for 24 h, 72 h, and 1 week displayed lower levels of cell death similar to untreated neurons (FIG. 14B-D). This was further supported by live/dead staining of neurons with calcein and propidium iodide (PI) 72 h after treatment (FIGS. 15A and B). All PA-treated conditions had a similar ratio of live cells to total cells compared to nontreated controls and a significantly higher ratio than a lysis-buffer treated dead control (FIGS. 15A and B).

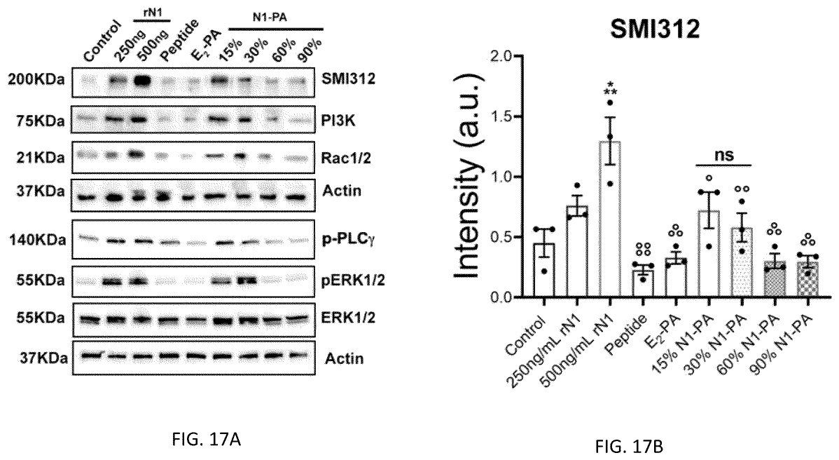

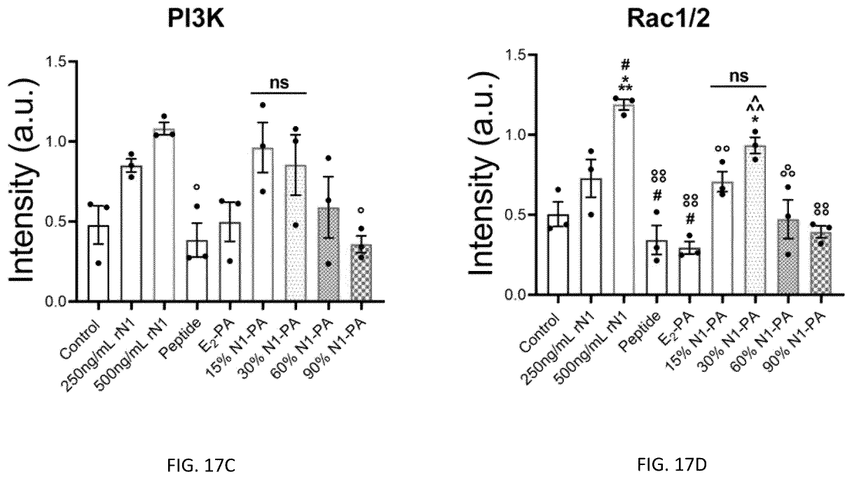

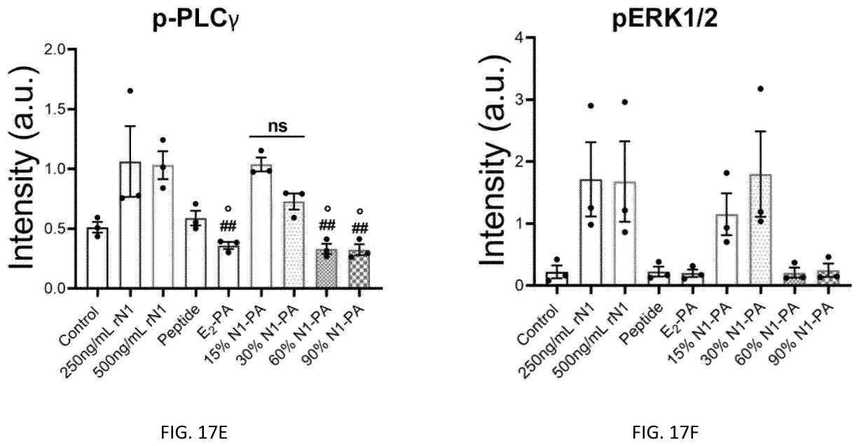

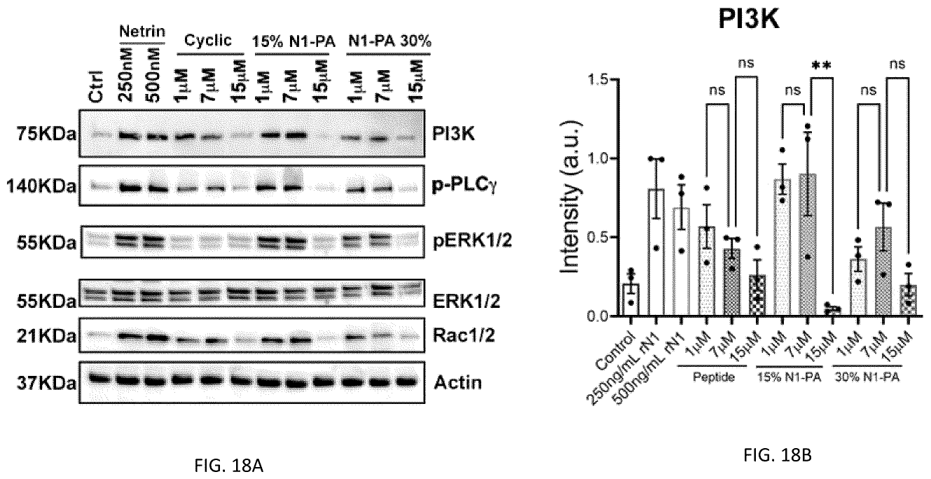

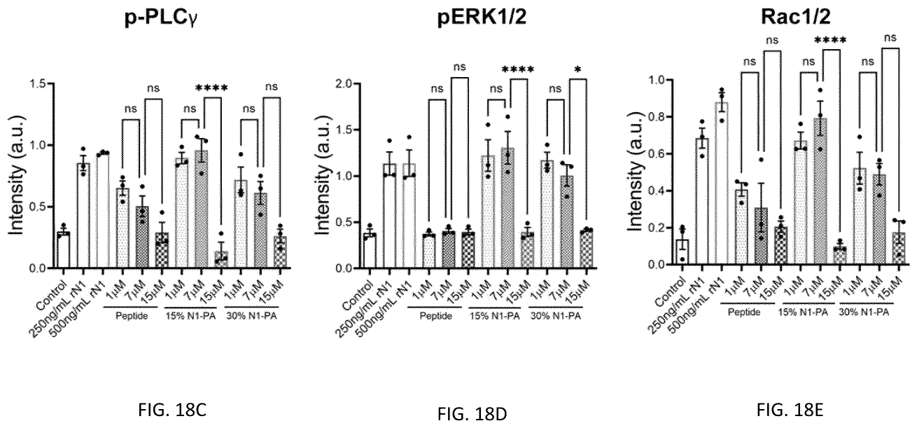

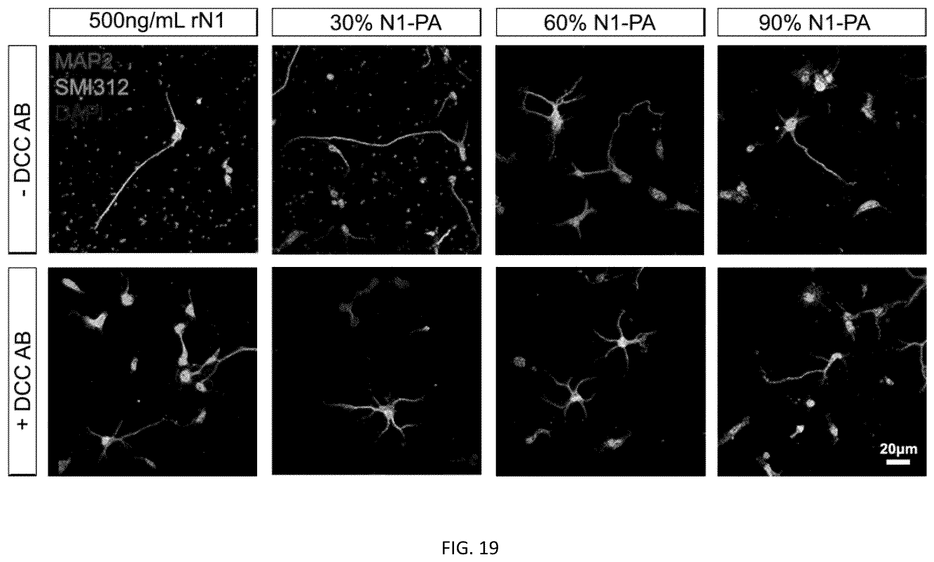

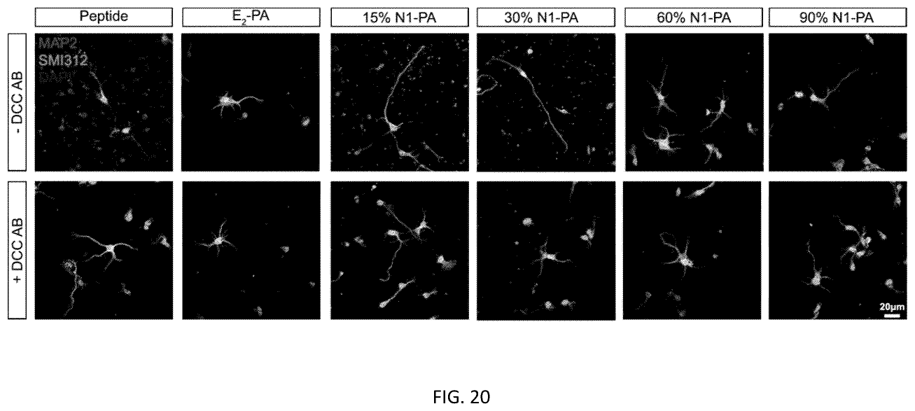

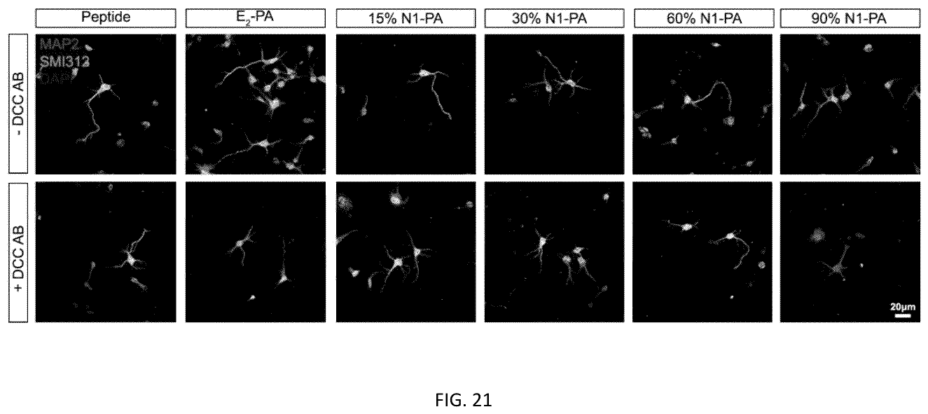

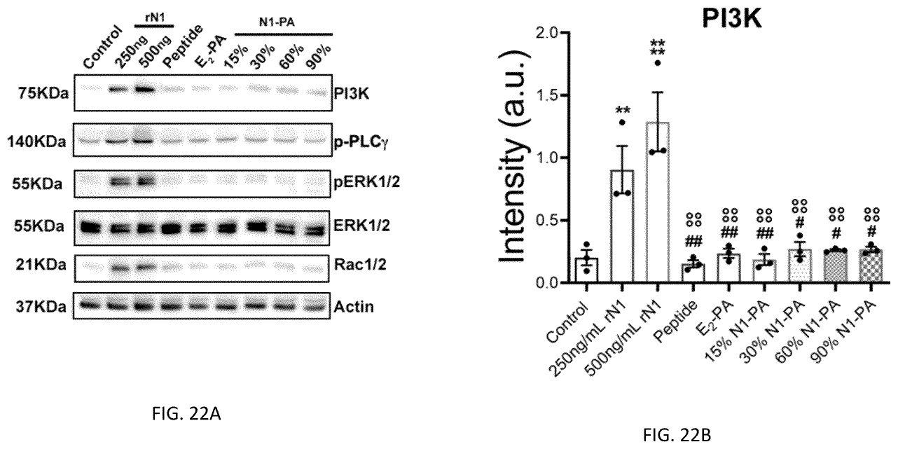

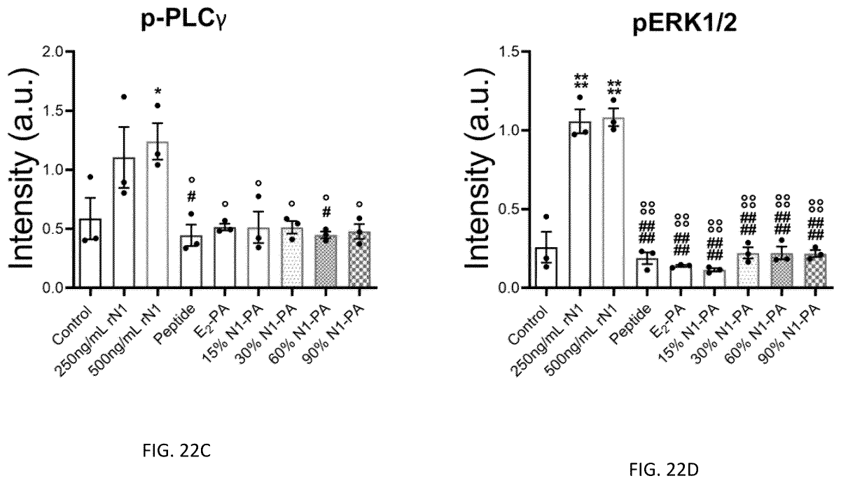

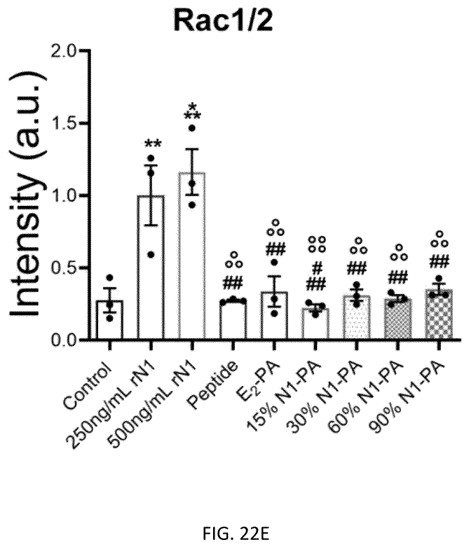

It was next examined whether any N1-PA assemblies could successfully activate the DCC pathway. Using Western blot, the phosphorylation of several downstream intracellular substrates associated with DCC receptor activation, such as phosphoinositide 3-kinase (PI3K), phosphorylated phosphoinositide-specific phospholipase-γ (pPLCγ), phosphorylated extracellular signal-regulated kinase 1/2 (pERK1/2), and ras-related C3 botulinum toxin substrate 1/2 (Rac1/2), was investigated. PI3K and pPLCγ have both been implicated in axon guidance while ERK1/2 has been shown to specifically promote mitogen-activated protein kinase (MAPK) signaling upon DCC receptor activation, thus contributing to axon growth. Rac1/2 is required for DCC-dependent neurite outgrowth and specifically affects actin reorganization. Upregulation of the axonal marker SMI312 was also assessed, as differences in axon guidance and growth will correlate with higher SMI312 expression. The N1-PA co-assemblies (15%, 30%, 60%, and 90%) were added to the media (containing 1,7, and 15 μM PA molecules with the netrin-1 mimetic sequence) for 24 h and compared to the addition of rN1 at 250 (3.67 nM) and 500 ng/mL (7.33 nM) concentrations. The range of PA treatment concentrations in which an approximate 2000-fold increase of bioactive PA to native protein was applied. Neurons treated with 15% or 30% N1-PA at 1 μM (FIGS. 16A and B) and 7 μM (FIG. 17A-F) showed some upregulation of the intracellular markers PI3K, pPLCγ, pERK1/2, Rac1/2, and the axonal marker SMI312 compared to the control and comparable to levels induced by rN1. Comparing between 1 and 7 μM treatments of 15% or 30% N1-PA, we did not find any significant differences in the activation levels of PI3K, pPLCγ, pERK1/2, or Rac1/2. This was further corroborated by observations of SMI312-labeled primary axons from neurons treated for 24 h (FIGS. 18-20). However, 60% and 90% N1-PA at the same 1 and 7 μM concentrations did not show any significant upregulation of these markers. These results are consistent with the differences observed by CG-MD simulations and TEM/SAXS (FIG. 1D-G), suggesting that the incorporation of N1-PA molecules in the supramolecular nanostructures is critical for bioactivity. Interestingly, at 15 μM, no N1-PA co-assemblies showed any upregulation of DCC receptor pathway markers (FIGS. 21 and 22), indicating that signaling is not effective at high N1-PA monomer concentrations. Considering the CG-MD simulation, TEM/SAXS, Western blot, and axon length analyses, the 15% N1-PA at 1 μM treatment was ultimately selected for further assessment.

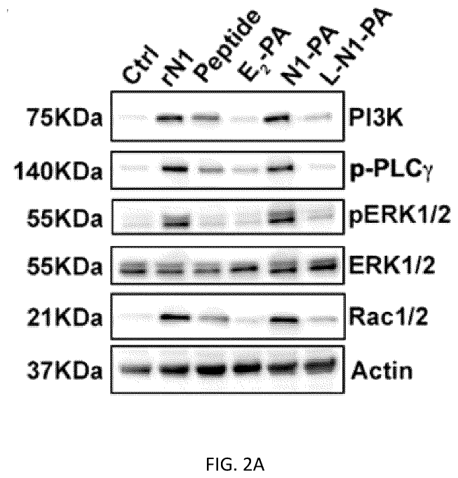

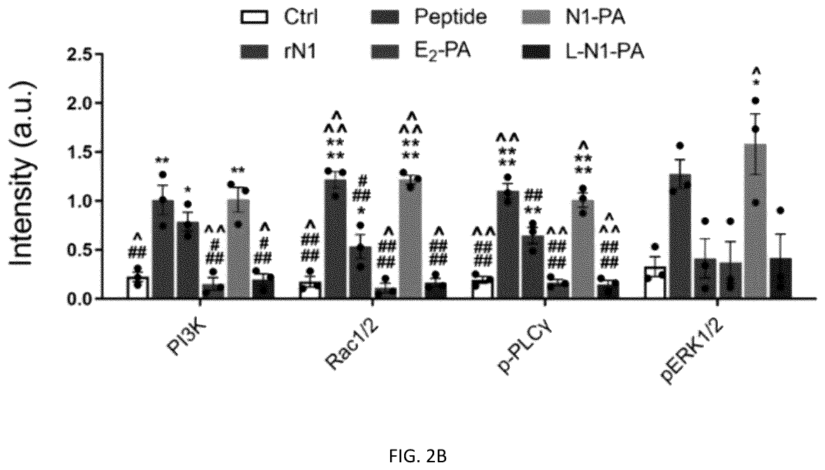

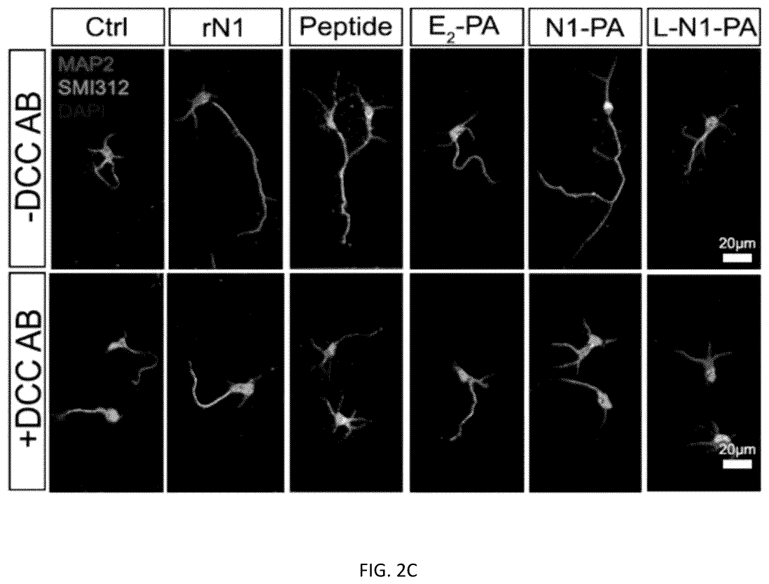

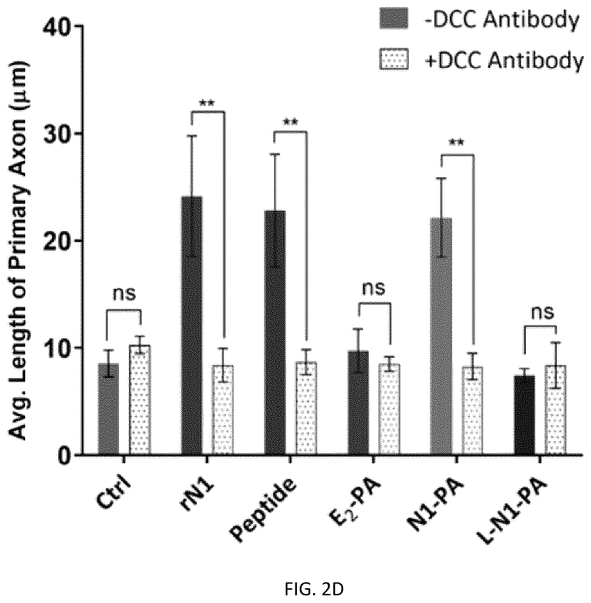

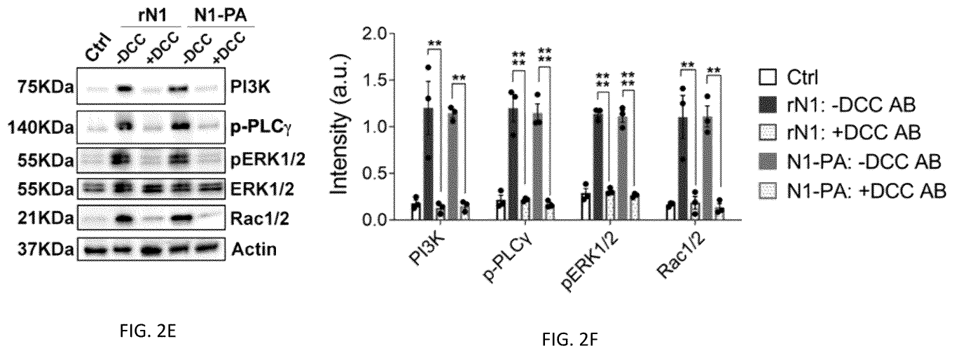

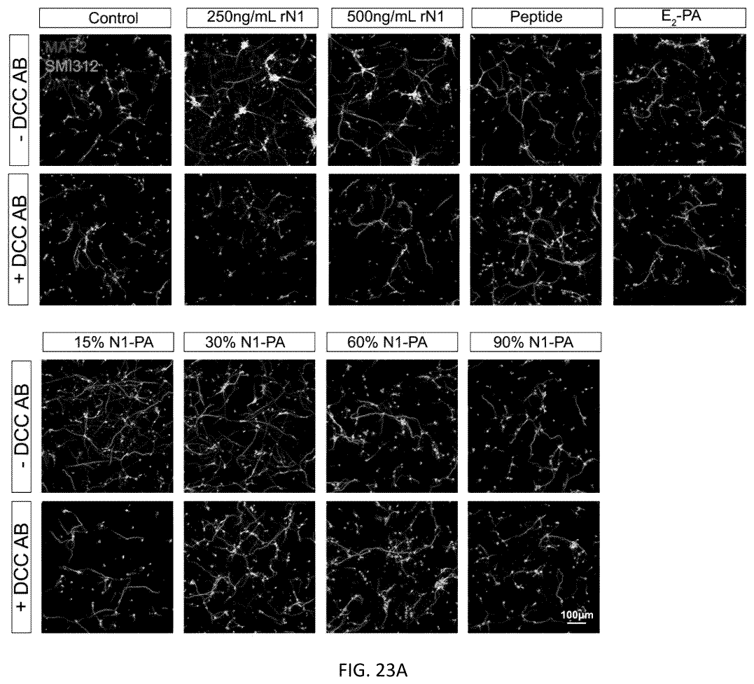

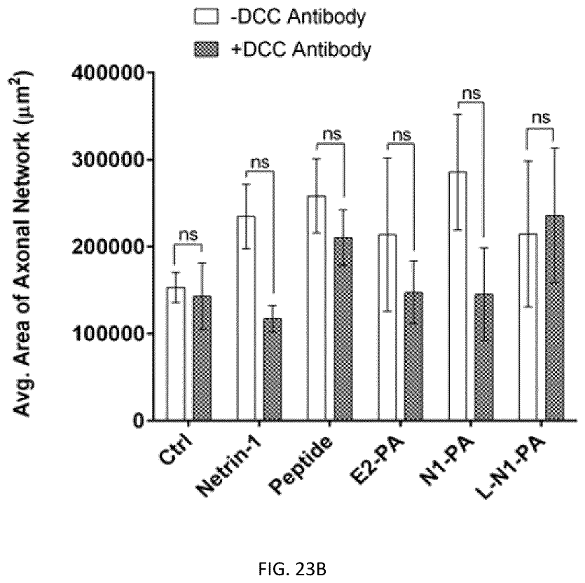

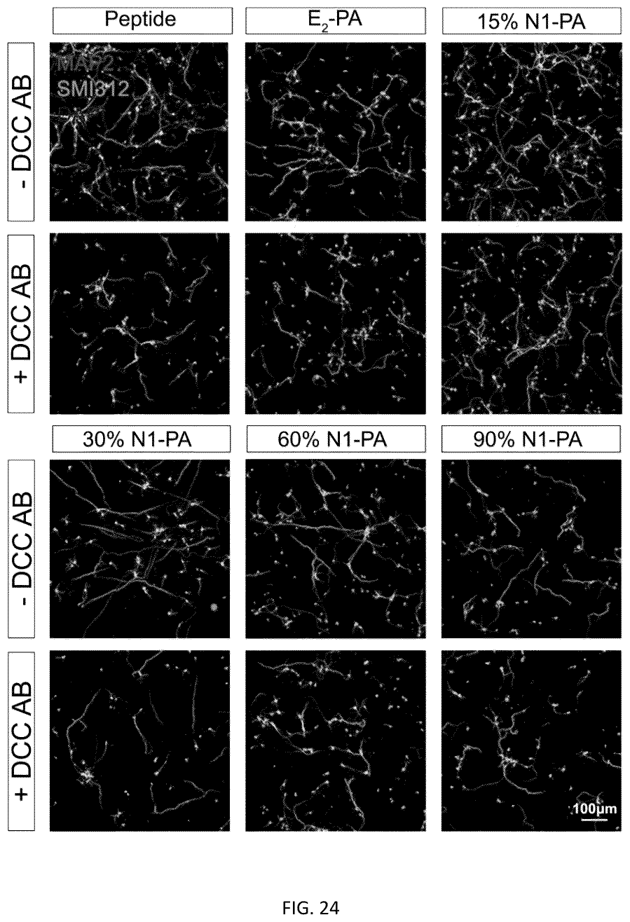

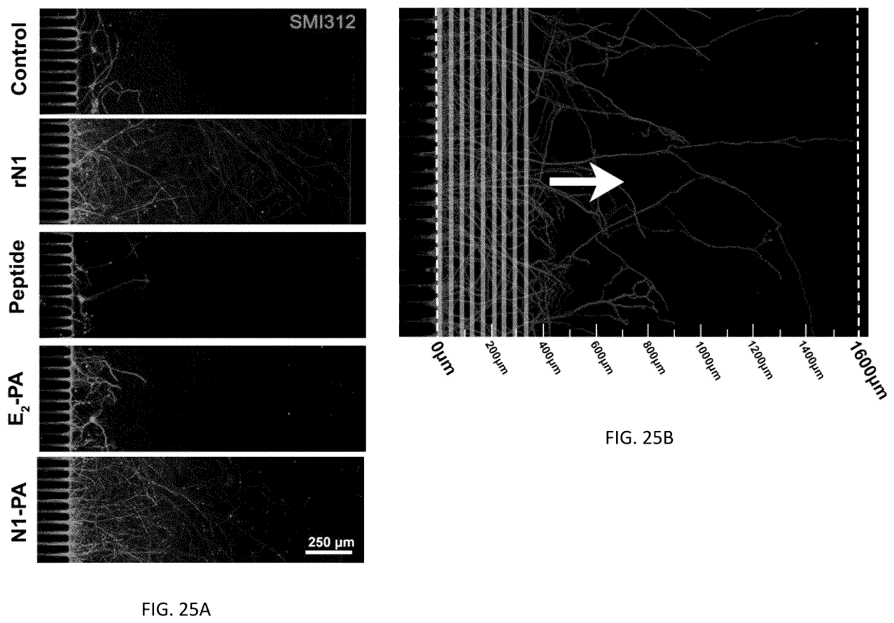

With the optimal N1-PA nanofiber treatment, it was confirmed that identical treatments with nonbioactive E₂-PA and L-N1-PA would not induce similar levels of activation. Neurons treated with E₂-PA for 24 h did not reveal any receptor activation, supporting the hypothesis that the cyclic CIDPC sequence, rather than the PA structure alone, induces the activation of the DCC receptor pathway (FIGS. 2A and B). Furthermore, L-N1-PA also failed to induce activation, highlighting the importance of the cyclic conformation of the IDP sequence. While the CIDPC peptide sequence alone caused some significant upregulation of PI3K, Rac1/2, and p-PLCγ in comparison to a control without any treatment, the effect was more limited than that of the N1-PA, suggesting that incorporation of molecules containing the CIDPC sequence to the PA fiber enhances bioactivity. To determine whether the differences in pathway activation were DCC receptor specific, the extracellular DCC receptor was blocked in the cell membranes of neurons by treating them with anti-DCC receptor antibody. It was hypothesized that receptor activation induced by rN1, the CIDPC cyclic peptide, and N1-PA nanofiber treatments would be significantly reduced in the presence of the anti-DCC-receptor antibody. The primary axon length of neurons treated for 24 h, both with and without the presence of the anti-DCC-receptor antibody in the media (FIGS. 2C and D), was first examined. In the presence of the anti-DCC receptor antibody, the primary axon length was significantly decreased in the rN1, cyclic peptide, and N1-PA conditions, indicating DCC receptor-specific intracellular pathway activation (FIGS. 2C and D). This experiment was also performed after 72 h, where similar trends were observed (FIGS. 23 and 24). It was then tested whether the application of the anti-DCC receptor antibody would block the upregulation of intracellular markers PI3K, p-PLCγ, ERK1/2, and Rac1/2, markers that were previously upregulated by rN1 and N1-PA (FIGS. 2E and F). As hypothesized, the anti-DCC receptor antibody significantly downregulated each intracellular substrate examined for both rN1 and the N1-PA nanostructures. This evidence suggested that both N1-PA supramolecular nanostructures and rN1 induced changes in both DCC receptor pathway activation and neuronal morphology through specific interactions with the DCC receptor.

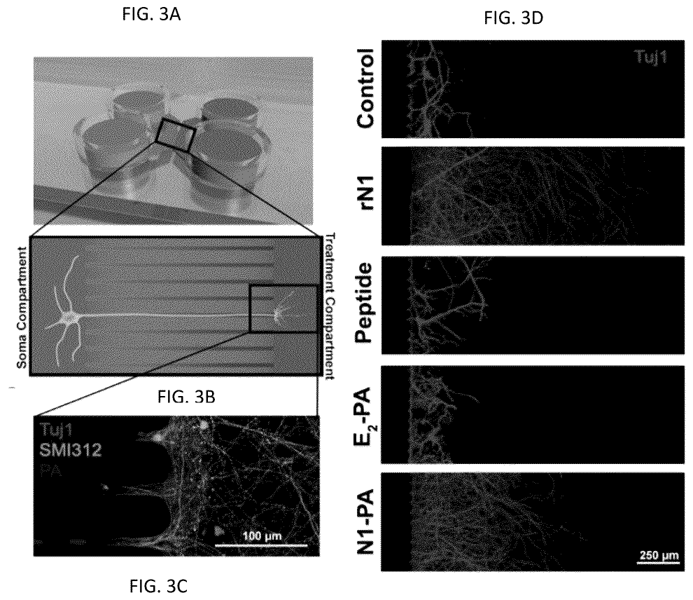

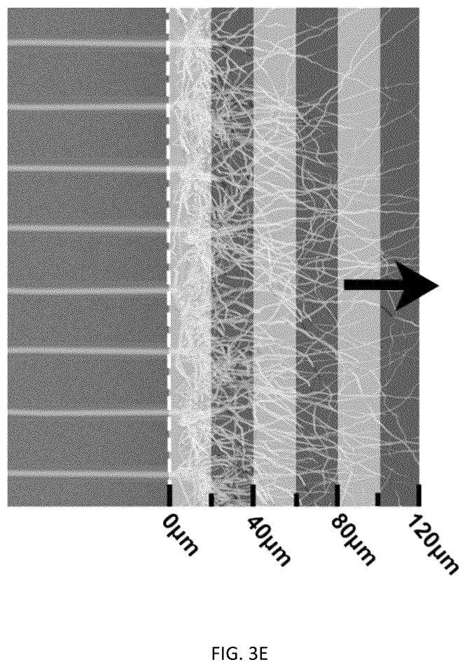

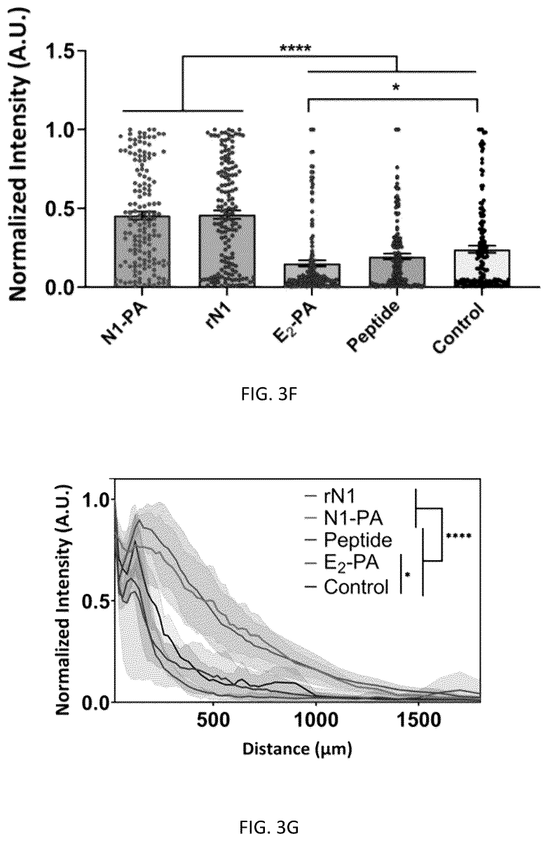

Measuring Neurite Outgrowth. Netrin-1 is typically presented to neurons during development in a gradient that attracts neurites to different locations in the CNS. To model this in vitro, a microfluidic device system utilizing two main compartments separated by a series of microchannels, a soma compartment (shown in blue in FIGS. 3A and B) and a treatment compartment (shown in green in FIGS. 3A and B), was used. The width of the microchannels is large enough to allow neurites to grow through but narrow enough to prevent neuronal somas from migrating into the treatment compartment (FIG. 3B). Primary neurons were seeded into the device and treated them with rN1, E₂-PA, N1-PA, or cyclic peptide for 1 week. For imaging by confocal microscopy, neurites were labeled with class III β-tubulin (Tuj1) and axons with the marker SMI312, and the PA materials were nonspecifically stained with DAPI (FIGS. 3C and 25A). It was confirmed in the microfluidic device experiments that neurites crossed into the treatment chamber under all conditions after 1 week (confocal microscopy confirmed the presence of PA in the treatment compartment). Images revealed qualitatively that compartments containing rN1 and N1-PA nanofibers exhibited the greatest infiltration of neurites relative to other conditions (FIG. 3D). To quantify and confirm these observations, the normalized Tuj1 intensity was calculated at regular intervals along the length of the treatment compartment (FIGS. 3E and F and 25B). rN1 and N1-PA both showed significant levels of normalized intensity as well as the longest distance compared to the E₂-PA, the cyclic peptide, and a control without any treatment (FIG. 3G). Neurons treated with cyclic peptide performed similarly to the control, while E₂-PA performed slightly worse. As discussed above, the CIDPC cyclic peptide has only a modest effect on neurons 24 h after treatment, less than that of rN1 and N1-PA, as confirmed by both Western blot and morphometric analysis. It is possible that the half-life of the CIDPC cyclic peptide over the course of 1 week, despite multiple treatments during the course of the experiment, limits its overall efficacy. E₂-PA, which does not contain a bioactive component, similarly had no discernible effect on neurite outgrowth.

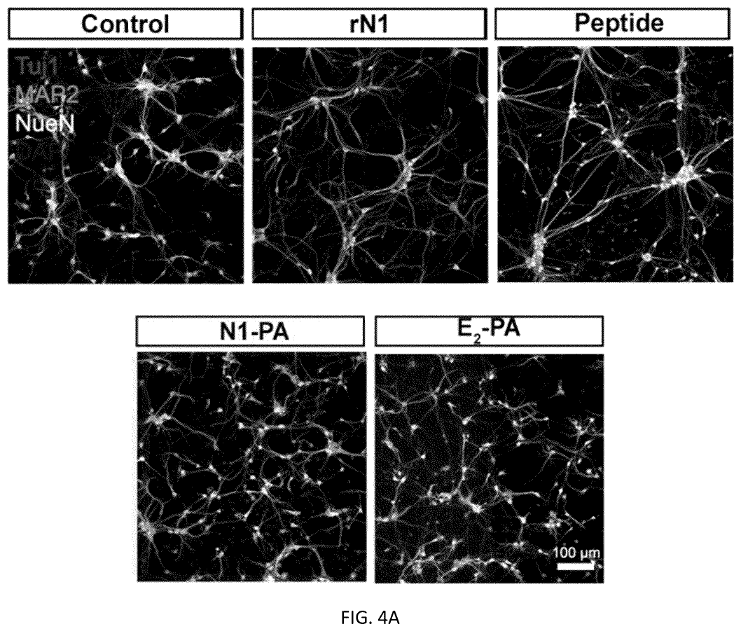

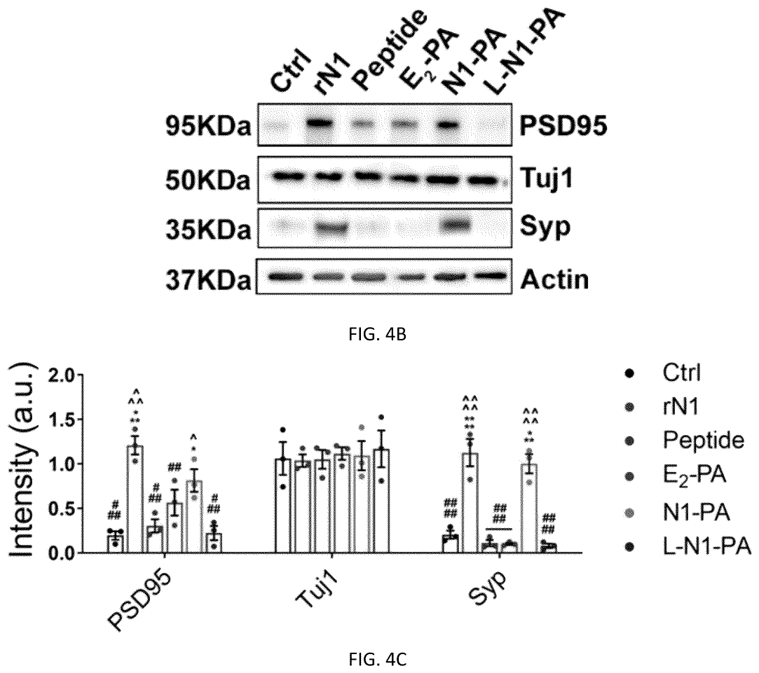

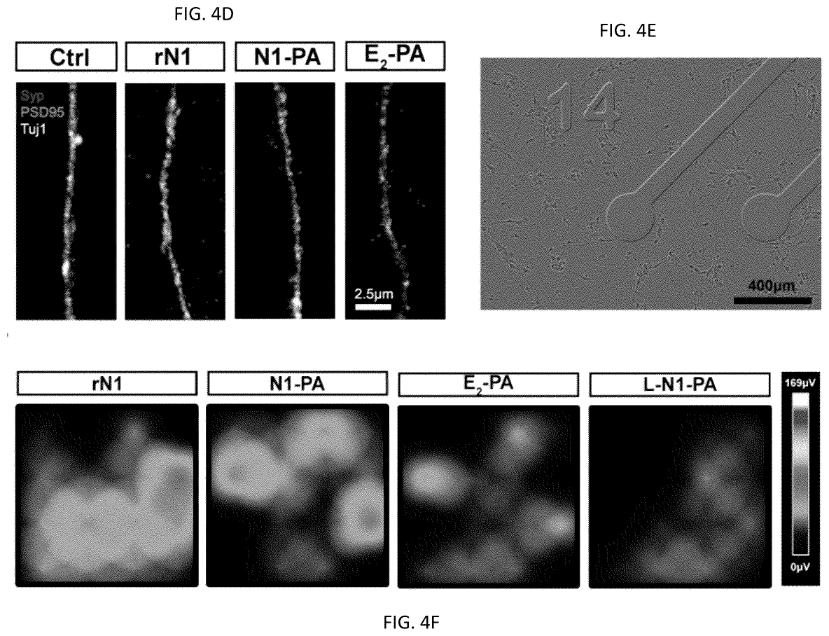

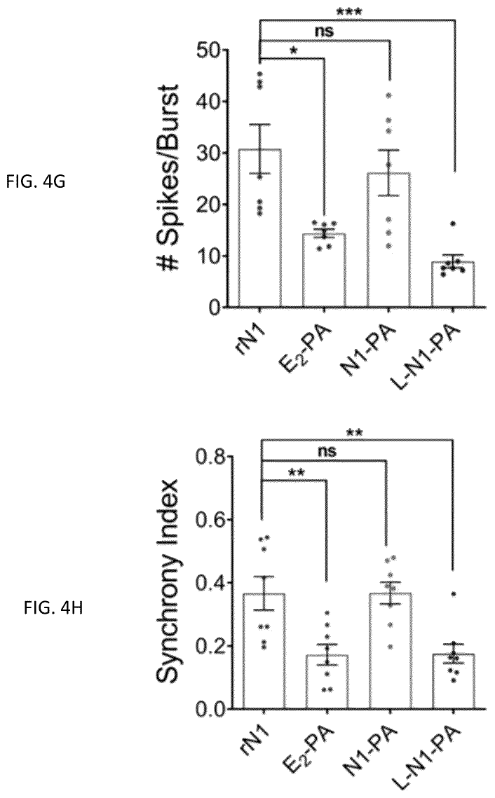

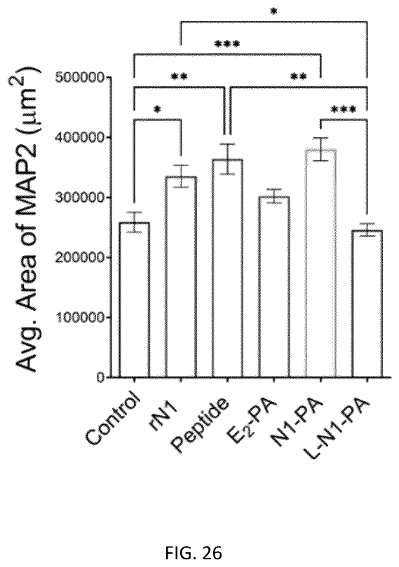

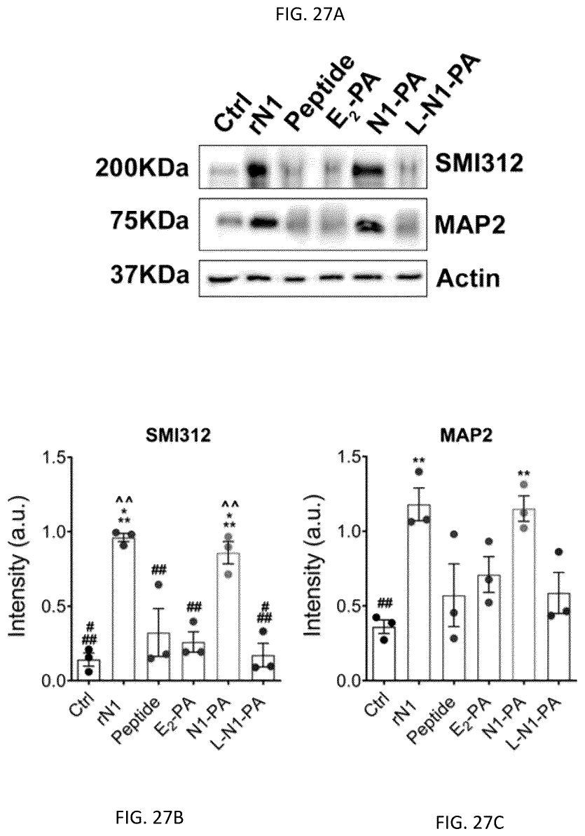

N1-PA Influences Neuron Synaptogenesis and Functional Electrical Activity. In addition to influencing neurite behavior, netrin-1 also induces functional maturation through morphological changes and increased synaptogenesis, which ultimately affect the functional activity of neurons. To evaluate whether N1-PA supramolecular nanofibers might have a similar effect biochemical and imaging experiments were conducted with primary neurons treated with the nanostructures over the course of two weeks in vitro (FIG. 4A). Interestingly, neurons treated with either the N1-PA or E₂-PA nanofibers were significantly more uniformly distributed and less clumped than neurons treated with either rN1 or a cyclic peptide or not exposed to any treatment (control). Differences in the expression of the dendritic marker microtubule-associated protein 2 (MAP2), associated with mature neuronal cultures, were observed by quantifying the average area of MAP2 for each condition. Cyclic peptide- and N1-PA nanofiber-treated neurons resulted in similar areas of MAP2 coverage as rN1-treated neurons. These three conditions had significantly larger areas of MAP2 than the control- and L-N1-PA nanofiber-treated neurons, indicating a more mature neuronal phenotype (FIG. 26). The expression of MAP2 as well as the axonal marker SMI312 was confirmed by Western blot analysis of neurons treated for 2 weeks in vitro (FIG. 27A-C). These results suggested that N1-PA affects the maturation of developing neurons.

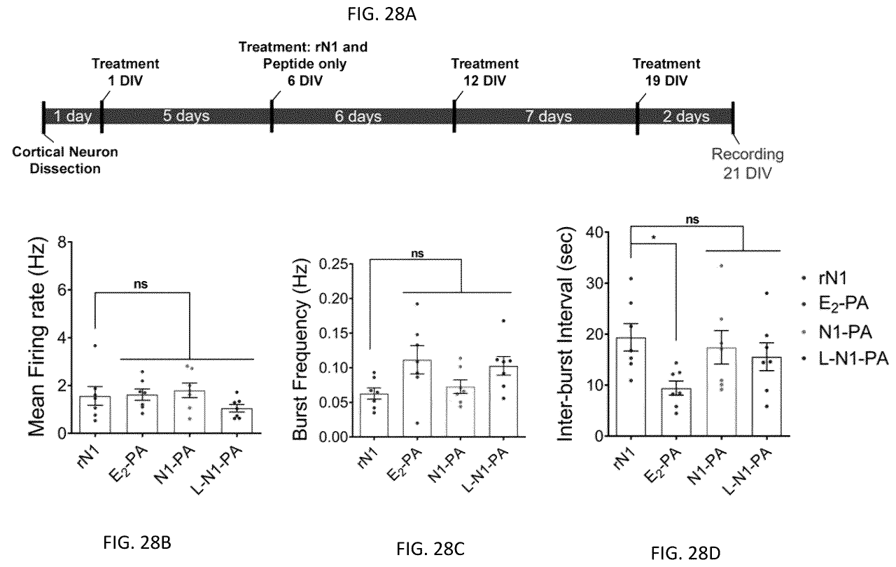

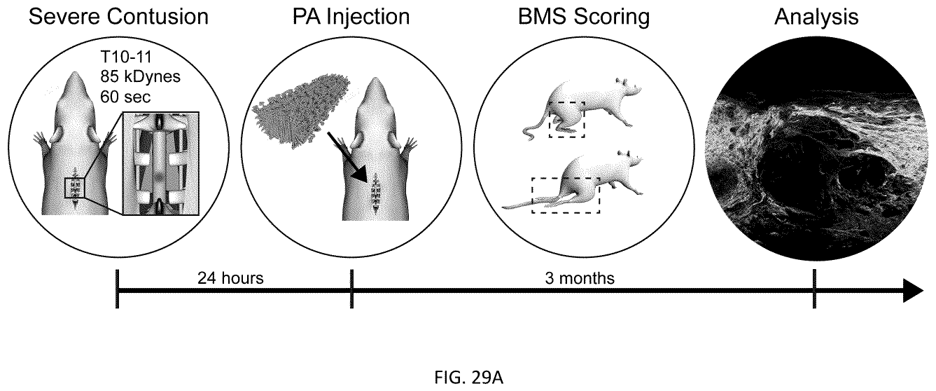

Next, both synaptic protein expression and functional electrical activity of neurons treated with N1-PA supramolecular nanofibers was evaluated to determine whether fiber-bound netrin-1 mimetic could influence synaptogenesis similarly to the native netrin-1 protein. Using isolated whole-cell extracts, the amount of the presynaptic marker synaptophysin (Syp) and the postsynaptic protein PSD95 were quantified (FIG. 4B-D). Neurons treated with rN1 and N1-PA nanofibers expressed significantly higher levels of Syp and PSD95 compared to the control, cyclic peptide, and E₂-PA and L-N1-PA nanostructures (FIGS. 4B and C). Confocal images of pre- and postsynaptic vesicles showed similar trends for neurons treated with rN1 and N1-PA nanofibers, demonstrating that higher levels of these proteins were properly distributed along the neurites (FIG. 4D). To determine whether the increased synaptic protein expression correlated with more functional electrical activity of neuronal networks, neurons treated with PA nanostructures were evaluated in solution using a multielectrode array (MEA) plate. Primary neurons were seeded on an Axion Biosystems 48-well plate and treated over the course of 21 days (FIGS. 4E and 28A). Each well contained 16 PEDOT electrodes that collected spontaneous network activity during 5 min measurements (FIG. 4F). After 21 days, parameters that indicate the functional maturity of neurons such as mean firing rate, synchrony index, number of spikes per burst, burst frequency, and interburst interval were evaluated (FIGS. 4G and H and 28B-D). rN1 and N1-PA nanofiber conditions outperformed L-N1-PA and E₂-PA nanofibers in the number of spikes per burst and the synchrony index, a metric that describes the ability of a single neuron to electrically and coordinately stimulate neighboring neurons (FIGS. 4G and H). An increase in these parameters indicates neural network complexity and maturation. However, no significant differences in the mean firing rate, burst frequency, or interburst interval measurements were observed among conditions (FIGS. 28B and C). Taken together, these data demonstrate that N1-PA nanofibers enhance synaptogenesis and functional maturation in neuronal cultures to a similar extent as rN1 protein.

In primary cortical neurons the N1-PA supramolecular nanofibers significantly upregulated the expression of intracellular proteins associated with DCC receptor activation and increased neurite growth similar to the native netrin-1 protein. Additional in vitro experiments utilizing microfluidic devices further highlighted the ability of N1-PA nanofibers to enhance neurite growth. Furthermore, N1-PA nanofibers were able to enhance maturation, as indicated by increased MAP2 and synaptic protein expression as well as changes in the functional electrical activity. Notably, the N1-PA monomer was most bioactive when incorporated at low concentrations in filamentous assemblies, outperforming both linear N1-PA controls and the free cyclic peptide. This emphasizes the importance of cyclic netrin-1 mimetic presentation in a nanostructure for cell signaling.

### PA Treatments Modulate the Inflammatory Response Following SCI

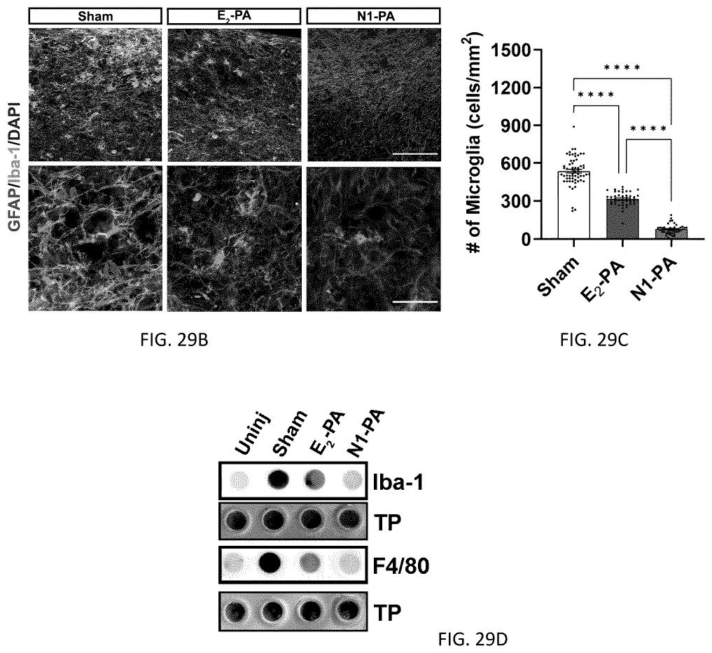

To determine whether netrin-1 mimetic PA could influence cell behavior and improve functional recovery in vivo, E₂-PA and N1-PA were evaluated in a severe contusion mouse SCI model. 8-10-week-old CD1 mice were severely contused at the T10-T11 level of the spinal cord with a force of 85 kDynes and a dwell time of 60 secs (FIG. 29A). After 12 weeks, spinal cord tissue was harvested and the effect of PA treatment on the inflammatory response as well as neuron growth and survival was evaluated. Both microglial and macrophagic markers surrounding the lesion site were evaluated 12 weeks after contusive injury. First, the number of Iba-1 (ionized calcium-binding adaptor molecule 1) positive microglia and macrophages 12 weeks after injury was evaluated by microscopy (FIG. 29B). Qualitatively, a noticeable decrease in the number of Iba-1 positive cells was observed in E2-PA and N1-PA treated conditions compared to sham control, with the most dramatic downregulation in N1-PA-containing PA treated tissue (FIG. 29B). Quantification confirmed that N1-PA treated mice had the lowest average number of Iba-1 positive cells, significantly lower than both the sham and E2-PA treated condition (FIG. 29C). A dot blot analysis of injured spinal cord tissue corroborated these results (FIG. 29D). Both Iba-1 and F4/F80, a membrane-bound glycoprotein expressed on both macrophages and microglia, were downregulated in all PA treated conditions but most notably in N1-PA treated mice.

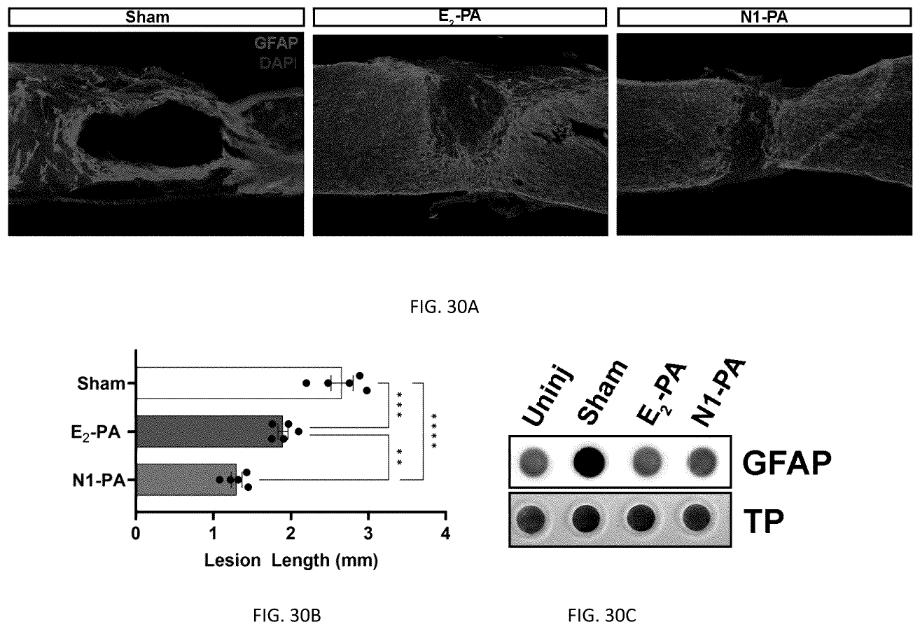

Following SCI, the glial scar, formed from reactive astrocytes and chemorepellent protein factors, prevents the propagation of injury to healthy tissue. However, it also prevents neuron regrowth back into the lesion site. One strategy to improve recovery following SCI is to reduce the amount of glial scarring As such, it was next evaluated whether netrin-1 mimetic PA systems could also modulate glial scar formation in vivo. 12 weeks following treatment, differences in the amount GFAP, an astrocytic marker, surrounding the lesion site as well as the total length of the lesion, defined by the glial scar border, of SCIs treated with saline (sham), E₂-PA, and N1-PA was assessed (FIG. 30A-B). Qualitative observations for the sham condition revealed a thick GFAP positive astrocytic border surrounding the lesion site, while PA treated spinal cords showed a more disperse astrocytic lesion border (FIG. 30A). A dot blot analysis of GFAP protein expression supported this observation, with sham treated mice expressing a higher level of GFAP than the uninjured and PA treated groups (FIG. 30C). Interestingly, PA treatments also reduced the length of the lesion site compared to the sham control, with N1-PA most significantly reducing the lesion length (FIG. 30B). Without wishing to be bound by theory, as astrocytes can express the DCC receptor, the receptor that the N1-PA interacts with in neurons, the N1-PA may influence astrocytic behavior via a similar mechanism. While not as significantly effective as netrin-1 mimetic containing PA treatments, the E₂-PA also reduced GFAP expression and the size of the lesion. This bioactivity may be attributed to the interaction of astrocytes with non-specific proteins and ions electrostatically bound to the negative exteriors of the E₂-PA nanofibers.

PA Supramolecular Structures Stimulate Neurite Regrowth into Lesion Site

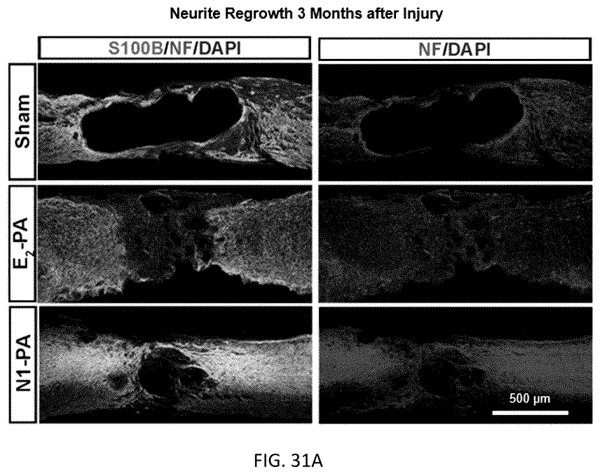

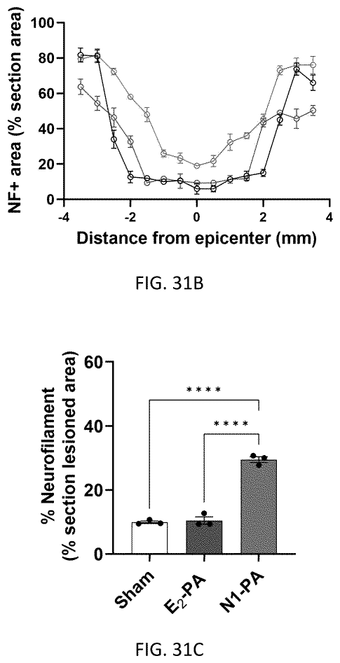

Longitudinal sections of the contused spinal cord tissue were evaluated for differences in the amount of neurofilament (NF), a neuronal cytoskeletal marker, present in the lesion site 12 weeks after injury (FIG. 31A). N1-PA treated spinal cords had increased amounts of neurite infiltration into the lesion site in comparison to both E₂-PA and saline. To quantify the extent of this growth, the percentage of NF-positive area at various points throughout the length of the lesion was measured (FIG. 31B). N1-PA had elevated levels of NF-positive area throughout the entirety of the injury site, from the proximal to distal border, when compared to both the non-bioactive E₂-PA and sham conditions. Similar trends were observed when considering the total percentage of NF present in the entirety of the lesion, which showed significant differences between conditions (FIG. 31C). These results suggest that netrin-1 mimetic containing PA assemblies stimulate neurite regrowth in vivo following injury.

### Reduction in Tissue Degeneration Distal to Injury Site in N1-PA Treated Spinal Cords

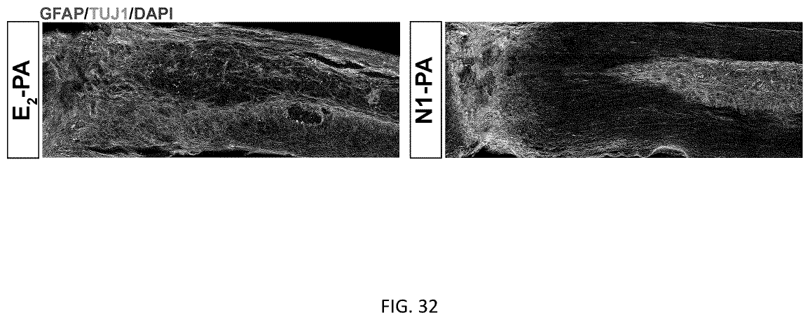

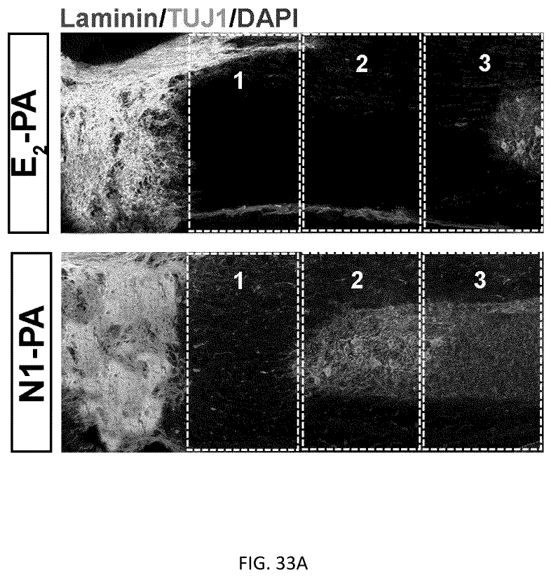

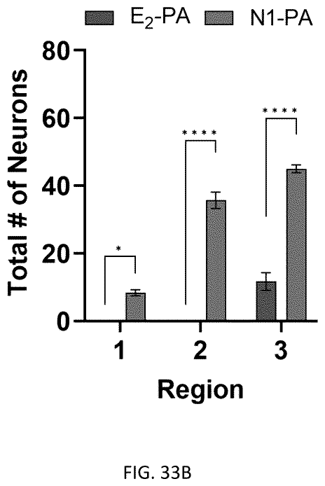

Next it was evaluated whether N1-PA would reduce tissue and neuron degeneration distal to the lesion site. First, the integrity of tissue labeled with GFAP (an astrocytic marker), Tuj1 (a neural cytoskeletal maker), and DAPI (a nuclear marker) was evaluated (FIG. 32). Large patches of tissue cavitation were observed in the E₂-PA treated condition, apparent from the lack of GFAP and DAPI staining, in addition to minimal Tuj1 expression, indicating a lack of neuronal cell bodies. In contrast, the N1-PA condition showed no significant signs of tissue cavitation. Interestingly, the N1-PA condition had a higher number of Tuj1 positive neurons in regions closer to the lesion site (FIG. 33A). To quantify this observation, the spinal cord was subdivided into three distinct sections distal to the injury site and the total number of Tuj1-positive neuronal cell bodies present in each region was counted (FIG. 33B). N1-PA treated spinal cords had significantly more neurons closer to the original lesion site, indicated by a great number of Tuj1 positive cells in regions 2 and 3.

These results demonstrate that the netrin-1 mimetic sequence presented on the surface of N1-PA supramolecular nanofibers prevents neuronal cell death following SCI. More specifically, the netrin-1 mimetic peptide on the N1-PA likely interacts with DCC receptors expressed on neuronal bodies present in the spinal cord, effectively blocking apoptotic intracellular activation pathways.

### PA Supramolecular Structures Improve Functional Recovery Following Severe Spinal Contusion

It was next evaluated whether N1-PA would promote functional recovery (e.g. motor improvements) after spinal cord injury. To determine whether netrin-1 mimetic PA supramolecular assemblies could improve functional recovery and neurite regrowth in vivo, E₂-PA and N1-PA were evaluated in a severe contusion mouse SCI model. As described above, 8-10-week-old CD1 mice were severely contused at the T10-T11 level of the spinal cord with a force of 85 kDynes and a dwell time of 60 secs. 24 hours post-contusion, mice were screened for hind limb movement and were removed from the study if they received Basso mouse scale for locomotion (BMS) scores above 0. Mice with BMS scores of 0, which corresponds to no hind limb movement, were then injected with either saline, E₂-PA or N1-PA via stereotactic injection at the lesion site and evaluated weekly for hind limb movement over the course of 12 weeks.

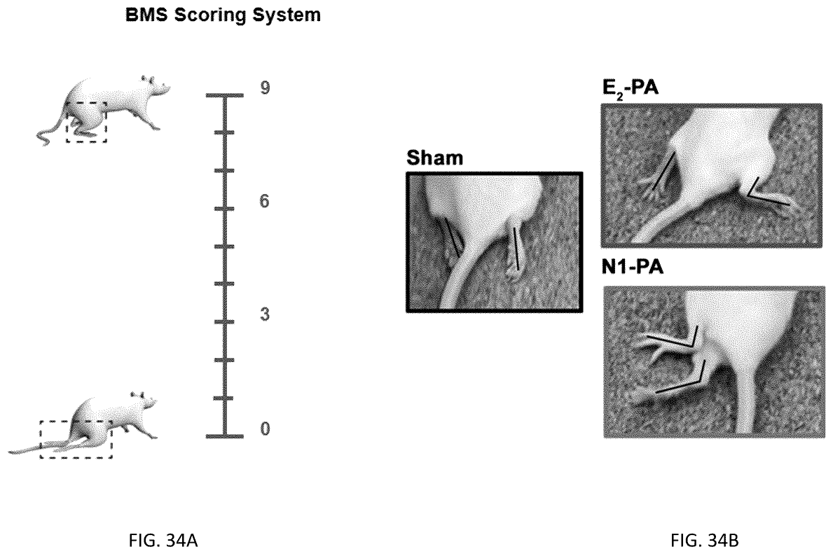

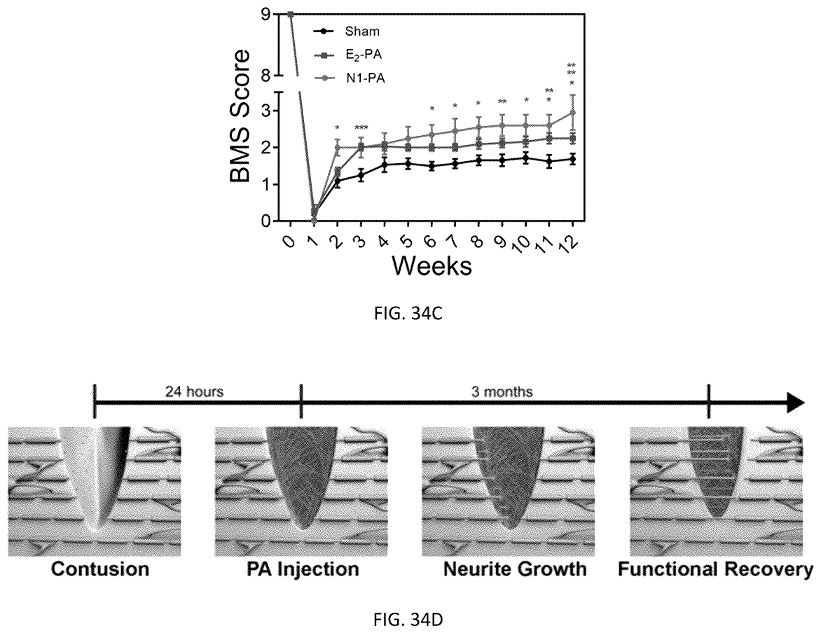

A schematic of the BMS Scoring system is shown in FIG. 34A. Results are shown in FIG. 34C. At 2 weeks, mice treated with N1-PA had significant improvements in BMS scores compared to sham control, with significantly improved BMS scores observed at each of the 6, 7, 7, 9, 10, 11, and 12 week time points. After 12 weeks, N1-PA (n=5) achieved a BMS scores of 2.75±1.15, significantly higher than the sham control (n=16), which achieved a BMS score of 1.69±0.60. E₂-PA treated mice (n=16) also showed some significant improvement in comparison to the sham with a BMS score of 2.25±0.58, but to a lesser degree than the N1-PA conditions. Representative images of hind limbs for each group are shown in FIG. 34B.

Provided herein are supramolecular nanostructures that mimic the bioactivity of neurotrophic factor netrin-1, a contributor in development and regenerative functions following injury in the adult central nervous system. The bioactive element in the supramolecular nanoscale assemblies was a peptide amphiphile molecule containing at one terminus a cyclic peptide structure with the capacity to activate the DCC receptor of the native protein netrin-1. Imaging and computer simulations showed that the bioactive netrin-1 mimetic molecules can only be incorporated in low concentrations into filamentous assemblies and, interestingly, bioactivity judged by receptor activation is diminished at high concentrations. This demonstrates the importance of the one-dimensional nanostructure in achieving efficient cell signaling relative to free monomer or small aggregates of the cyclic peptide amphiphile. Using primary cortical neurons, only fibrous netrin-1 mimetic supramolecular assemblies increased neurite growth and functional maturation, similar to the native netrin-1 protein. Neurons cultured in a microfluidic device displayed significant neurite outgrowth through a netrin-1 mimetic nanofiber-treated compartment. In addition, neurons treated with the fibrous bioactive supramolecular assemblies had increased synaptic protein expression, which correlated with changes in functional electrical activity parameters such as the number of spikes per burst and the synchrony index. Furthermore, in-vivo experiments demonstrated that treatment with N1-PA following spinal cord injury reduced inflammation, reduced glial scar formation, stimulated neurite regrowth into the lesion site, and improved functional recovery from SCI.

Given the challenges in using native proteins as therapies such as short half-lives and therefore costly high dosages, the biomimetic nanoscale supramolecular assemblies have potential as future therapies to treat injuries or disease in the central nervous system. Netrin-1 mimetic nanostructures could be incorporated into supramolecular hydrogel systems to promote recovery following spinal cord injury by stimulating neuron growth and synaptogenesis, as demonstrated in vitro. These nanostructures can also be considered as a therapy for diseases known to be affected by the lack of netrin-1 upregulation, including Alzheimer's disease, Parkinson's disease, and stroke.

### Methods

PA Synthesis. PA molecules were synthesized using standard Fmoc solid-phase peptide chemistry on a Rink amide MBHA resin (EMD). Amino acid couplings were performed in a CEM Liberty Blue microwave-assisted peptide synthesizer (CEM, Matthews, NC, USA). Each amino acid was coupled using 4 mol equiv of protected amino acid, 8 mol equiv of ethyl cyanohydroxyiminoacetate (Oxyma), and 4 mol equiv of N,N′-diisopropylcarbodiimide (DIC) in N,N-dimethylformamide (DMF) for 2-4 min at 90° C. Palmitic acid (C₁₆) was conjugated to the N-terminus of the peptide using this same procedure. Fmoc groups were cleaved using 20% 4-methylpiperidine and 0. μM hydroxybenzotriazole (HOBt) in N,N-dimethylformamide (DMF) at 90° C. for 30 s. To cleave PA molecules off the resin and to deprotect amino acid side chains, a solution of 95:2.5:2.5 trifluoro-acetic acid (TFA)/triisopropylsilane (TIPS)/water was added to the reaction vessel for 2-3 h. For PA molecules containing cysteine, 5% DODT (2,2′-(ethylenedioxy)diethanethiol) was also included in the cleavage mix of 95:2.5:2.5 trifluoroacetic acid (TFA)/triisopropylsilane (TIPS)/water. Volatile solvents were concentrated with rotary evaporation before the PA molecules were precipitated with a cold diethyl ether. Crude PA material was dried on a fritted filter. PAs were purified by preparative-scale reverse-phase high-performance liquid chromatography (Shimadzu Prominence or Waters Prep 150) using a Phenomenex Gemini column (C-18 stationary phase, 5 μm, 100 Å pore size, either 30×150 mm or 50×250 mm). A mobile phase of acetonitrile and water was used, containing 0.1% NH₄OH if the sample was acidic or 0.1% TFA if the sample was basic. Pure fractions were identified using electrospray ionization mass spectrometry (ESI-MS) in the positive mode on an Agilent model 6520 Quadrupole Time-of-Flight (Q-ToF) using direct injection. MassHunter Work-station Data Acquisition software was used for instrument operation, and MassHunter Qualitative Analysis software was used for data analysis and processing. Excess acetonitrile was removed by rotary evaporation following purification. The samples were then freeze-dried, and the resulting PA powders were stored at −20 to −30° C. The purity of the PA molecules was confirmed using liquid chromatography-mass spectrometry (LC-MS).

Cyclization of CIDPC. Cyclization of the CIDPC sequence (via disulfide bond formation between the cysteines) for cyclized N1-PA and the cyclized CIDPC peptide was performed on purified and lyophilized C₁₆V₂A₂E₂G₂CIDPC₍ₗᵢₙₑₐᵣ₎ and CIDPC₍ₗᵢₙₑₐᵣ₎, respectively. Lyophilized C₁₆V₂A₂E₂G₂CIDPC₍ₗᵢₙₑₐᵣ₎ and CIDPC₍ₗᵢₙₑₐᵣ₎ were each dissolved at a concentration of 2 mg/mL in basic water containing 0.1% NH₄OH. To initiate the cyclization, dilute hydrogen peroxide (H₂O₂) (100 μL, 1% H₂O₂ for a 50 mg scale) was added. Upon completion of the cyclization reaction, N1-PA was purified by HPLC using a mobile phase of acetonitrile and water, both containing 0.1% NH₄OH. Cyclic CIDPC peptide was purified by HPLC using a mobile phase of acetonitrile and water, both containing 0.1% TFA. Pure fractions were identified using electrospray ionization (ESI) mass spectrometry and combined. Excess acetonitrile was removed by rotary evaporation. The samples were then freeze-dried, and the resulting PA powders were stored at −20 to −30° C. Synthesis schemes for N1-PA, uncyclized N1-PA, cyclic CIDPC peptide, and L-N1-PA are included in the Supporting Information under “Additional Synthesis Information” (Schemes S1-3).

PA Preparation. Lyophilized PA powder was dissolved in a 150 mM NaCl and 3 mM KCl solution at a concentration of 1 mg/100 μL (1 wt %). 1 μM NaOH (Millipore Sigma, cat. no. 109137) was added in 1-2 μL increments to pH adjust the sample to approximately 7.4-7.6. The pH was monitored using Fisherbrand pH test paper (cat. no. 13-640-502). The PA solution was then horn sonicated at a 10% amplitude for approximately 10 s. Following sonication, the pH was again adjusted to approximately 7.4-7.6. For PA co-assemblies, volumes of each PA solution (calculated based on the final molar ratio of the desired molecules) were mixed and horn sonicated at a 10% amplitude for approximately 10 s. Final PA solutions were annealed at 80° C. for 30 min, followed by a slow cool of 1° C. per minute to 25° C. using a Bio-Rad T100 Thermal Cycler to remain consistent with prior studies.

### Coarse-Grained Molecular Dynamics (CG-MD) Simulations.

The atomistic coordinates of the PAs were created in Avogadro⁷⁰ and transformed to the MARTINI⁷¹⁻⁷⁴ resolution using a version of martinize.py⁷⁵ adapted to include the palmitoyl aliphatic tail.⁷⁶ Following previously published procedures, we used coiled coil as secondary structure and neutralized the first E residue (from the aliphatic tail) in both the E₂-PA and N1-PA.^{26,77,78} Thus, the final charge per PA is −1 for E₂-PA and −2 for N1-PA (due to the additional acid group in the epitope). The protocol consists of two steps. In the first step, an E₂-PA fiber formed by 300 molecules was placed in a cubic box (17.1×17.1×17.1 nm³) solvated with CG water and enough ions to neutralize the system. The final PA concentration was 100 mM, which is within the 10- to 25-fold increment commonly used in simulations to accelerate the self-assembly process.⁷⁹⁻⁸¹ E₂-PA fibers were equilibrated for 10 μs, running three independent simulations. In the second step, N1-PA molecules were added randomly around the equilibrated fiber to reach the final composition (Table S1) with a minimum allowance of 3 Å. The box size was kept constant in the fiber direction (12.2 nm) but extended in the x- and y-dimensions to keep the concentration constant at 100 mM (Table S1). The 100% N1-PA system consisted of 300 molecules randomly distributed in a 17.1 nm side box. The results shown are the average of three independent simulations. However, results with no proper incorporation (FIG. 8) were discarded, and new simulations were run to keep N=3.

All CG-MD simulations and analysis were performed in GROMACS 2020.4,⁸² and Visual Molecular Dynamics (VMD) was employed for visualizations.⁸³ Å cutoff of 1.1 nm was employed for intermolecular interactions using potential shift for Lennard-Jones and a reaction field with a dielectric constant of 15 for electrostatics.⁸⁴ All systems were minimized before equilibration until the forces in atoms converged below 2000 pN, with a maximum of 5000 steps that was never reached. All simulations used a 25 fs time step and were run for 100,000,000 steps. This corresponds to 10 μs effective time after applying the reported 4× scaling factor due to CG speed up.^{71,72} Simulations were run under an NPT ensemble with semi-isotropic conditions for all the simulations except for the 100% N1-PA, which used isotropic conditions. A V-rescale algorithm was employed for the temperature (303 K, τT=1 ps)⁸⁵ and Berendsen was used for the pressure (1 bar, τP=3 ps).⁸⁶ Aggregation propensity (AP) measurements of E₂-PA and N1-PA show that the systems reached equilibration within the simulations time (FIGS. 9A and B). Clustering was measured by considering a cutoff distance of 0.6 nm for the aliphatic tails of PAs in the same cluster (FIG. 9H), and the incorporated N1-PAs were measured by counting the number of aliphatic tails added to the fiber core. The water contacts of the PAs were calculated using the integration of the radial distribution function for the first solvation sphere of each backbone and aliphatic tail bead (FIGS. 9C and D) and applying the 4×x factor to convert CG water to water molecules. The hydration analysis measures the total loss of water from the N1-PA free system using the information on the water contacts (FIG. 9I). The radial distribution analyses were made using the SC3 bead of the C₁₆ tail of the E₂-PA as reference (FIG. 9E-G).

Transmission Electron Microscopy. PA solutions were prepared at a concentration of 1 wt % (1 mg/100 μL). Immediately prior to grid preparation, the PA solution was diluted to 0.1 wt %. Samples were drop cast onto glow discharged carbon coated (5-6 nm thick) copper grids (Electron Microscopy Sciences, cat no. 50-248-92) and then negatively stained with a 2 wt % solution of uranyl acetate (SPI Supplies, cat no. 02624-AB). Samples were imaged on a JEOL 1230 TEM located in the NUANCE/BioCryo facility at Northwestern University.

Dynamic Light Scattering. PA solutions were prepared at a concentration of 1 wt % (1 mg/100 μL). Immediately prior to DLS measurements, the PA solution was diluted to 0.1 wt % in Milli-Q water and vortexed. A Malvern Zetasizer Nano ZSP light scattering spectrometer (located in the ANTEC facility at Northwestern University) and a ZEN2112 quartz cuvette were used for all DLS measurements. After an initial equilibration time of 30 s, PA samples were measured 10 times for a duration of 10 s each time at a temperature of 25° C. This process was repeated in triplicate. The measurement angle for the measurements was a 1730 backscatter.

Small-angle and Wide-angle X-ray Scattering. PA samples were prepared at a concentration of 2 wt % (2 mg/100 μL). Samples were not diluted prior to data collection. X-ray scattering experiments were performed at the Argonne National Lab at Sector 5 IDD. Samples were measured in a flow-cell set up. All data were background subtracted prior to analysis. Slope measurements and curve fitting were performed with either Igor or SasView software. Slope measurements were obtained in the Guinier region between q values of 0.005 and 0.01 in the log-log plot for fibers and between q values of 0.015 and 0.03 for systems that formed irregular aggregates. FTIR. PA samples were prepared as previously described but in a 150 mM NaCl and 3 mM KCl D₂O solution at a concentration of 1 wt % (1 mg/100 μL). For pH adjustments, 1 μM NaOD was used. Samples were loaded between two CaF₂ windows prior to data collection. Data were collected in the transmission mode. Data were background subtracted prior to analysis. FT-IR was performed at the Integrated Molecular Structure Education and Research Center (IMSERC) at Northwestern University.

Ellman's Reagent Assay. PA solutions were prepared as previously described at a concentration of 1 wt % (1 mg/100 μL). Ellman's reagent (Sigma-Aldrich) was dissolved to a concentration of 200 μM with a 100 mM PBS buffer solution. Prepared PA solutions were mixed with Ellman's reagent solution to achieve a final PA concentration of 0.04 wt %. After a 15 min of incubation at room temperature, UV-vis measurements were collected at room temperature using the Ocean Optics QEPro USB4000 UV-vis spectrophotometer equipped with a DH-2000-Bal lamp with a 1 cm path length cuvette.

Glutathione Assay. A 500 μM solution of 100% N1-PA was prepared in 10 mM PBS and analyzed with liquid chromatography-mass spectrometry (LC-MS). 5 mM glutathione (5 mM) was then added to 100% N1-PA, and the sample was left to incubate for 48 h before analysis with LCMS. Samples were injected on a 1290 Infinity II UHPLC System (Agilent Technologies Inc., Santa Clara, California, USA) onto a Poroshell 120 EC-C18 column (1.9 μm, 50×2.1 mm) (Agilent Technologies Inc., Santa Clara, California, USA) for reverse-phase separation, which was maintained at 30° C. with a constant flow rate at 0.500 mL/min using a gradient of mobile phase A (water, 0.1% formic acid (v/v)) and mobile phase B (acetonitrile, 0.1% formic acid (v/v)). The gradient program was as follows: 0-1 min, 5% B; 1-8 min, 5-100% B; 8-10 min, hold at 100% B; 10-10.10 min, 100-5% B; 10.10-14 min, hold at 5% B. “MS-Only” positive ion mode acquisitions were utilized on an Agilent 6545 quadrupole time-of-flight mass spectrometer equipped with a JetStream ionization source (Agilent Technologies Inc., Santa Clara, California, USA). Representative compound chromatograms and mass spectra were generated using the “Find by Formula” algorithm. The adducts [M+H]⁺, [M+Na]⁺, [M+K]⁺, and [M+NH₄]⁺ in the positive ion mode were identified for each chemical formula and summed together to generate a compound chromatogram for each injection.

IACUC Standard Statement. All animal housing and procedures were performed in accordance with the Public Health Service Policy on the Humane Care and Use of Laboratory Animals. All procedures were approved by the Northwestern University Institutional Animal Care and Use Committee.

Media Preparation. Preplating media and media 1 were prepared the day of the dissection. Preplating media was prepared by adding 250 μL of penicillin/streptomycin (Sigma-Aldrich, cat no. P4333), 70 μL of L-glutamine (Gibco, cat. no. 25030081), 155 μL of sodium bicarbonate (Gibco, cat. no. 25080084), and 1.25 mL of NHS (Gibco, cat. no. 16-050-122) to 25 mL of neurobasal media (Gibco, cat. no. 21103049). Media 1 was prepared by adding 500 μL of penicillin/streptomycin, 125 μL of L-glutamine, 290 μL of sodium bicarbonate, 500 μL of NHS, and 500 μL of glutamate (370 μg/mL Milli-Q, Millipore Sigma, cat. no. 49621) to 50 mL of neurobasal media. Both media were then sterile filtered through a 0.22 μm membrane (Millipore, cat. no. S2GPU05RE, cat. no. SCGP00525) and then incubated at 5% CO₂ and 37° C. for at least 1 h. Immediately prior to addition to neuron culture, 1 mL of B27 supplement (Gibco, cat. no. 17504044) was added to media 1. Media 2 was prepared by adding 500 μL of penicillin/streptomycin, 125 μL of L-glutamine, and 290 μL of sodium bicarbonate to 50 mL of neurobasal media. The media was sterile-filtered and then incubated at 5% CO₂ and 37° C. for at least 1 h. Immediately prior to addition to the neuron culture, the B27 supplement was added.

Primary Mouse Cortical Neuron Dissection and Culture. Neurons were obtained from embryonic mouse brains. Briefly, a timed pregnant CD1 mouse (Charles River) was sacrificed by cervical dislocation, and the embryos were extracted at embryonic day 16 (E16). During the dissection, all tissue was maintained in ice-cold HBSS (Gibco, cat. no. 14-170-112) supplemented with 1% penicillin/streptomycin. Dissected cortices were transferred into 5 mL of a 0.25% Trypsin/EDTA (Gibco, cat. no. 25200072) solution with 250 μL of 4000 U/mL DNase I (Worthington Biochemical, cat. no. LS002006) for 10 min. After this, cortices were transferred to a 50 mL Falcon tube filled with 5 mL of preplating media and 250 μL of 4000 U/mL DNase I. Cortices were mechanically dissociated using a 1000 μL pipet. Thirty seconds after disassociation, the supernatant was removed and placed into another 50 mL Falcon tube. The cells were then centrifuged at 1000 rpm for 5 min. The supernatant was removed, and cells were then resuspended in 2 mL of fresh media. This was transferred into a cell culture treated Petri-dish filled with 8 mL of preplating media. This was incubated at 37° C. for 40 min to allow glial cells to attach to the surface of the plate to improve culture purity. Following the preplating, the suspended neurons were filtered through a 100 μm cell strainer (Corning, cat. no. CLS431752) into a 50 mL Falcon tube. An additional 2 mL of preplating media was filtered to retain as many cells as possible. The cells were centrifuged again at 1000 rpm for 5 min. The supernatant was removed, and the pellet was resuspended in 2 mL of media 1. A further dilution of 1:50 (cells: media) was prepared to count the number of cells extracted from the dissection. Cells were counted with a hemocytometer before being seeded for experiments. Cells were maintained in a cell incubator at 5% CO₂ and 37° C. Twenty-four h after seeding, all of media 1 was replaced with media 2. Then, half of the media was exchanged with fresh media 2 every 4-5 days so that the neurons remained in culture.

In Vitro Assays. Neurons were seeded onto poly-D-lysine (PDL) coated surfaces and treated with PA in solution for all in vitro assays. Glass coverslips or plastic tissue culture-treated well plates were incubated in a solution of 0.01 mg/mL poly-D-lysine hydrobromide (MW 70,000-150,000; Sigma-Aldrich, cat. no. P6407) prepared in Milli-Q water at 37° C. for at least 2-3 h. Surfaces were then washed twice with sterile Milli-Q water and left to dry overnight. PA solutions were prepared as previously described at a concentration of 1 wt % (1 mg/100 μL). Immediately prior to treatment, PA solutions were diluted to the desired treatment concentration in cell media and vortexed for 10 s. Concentrations of PA were calculated based on the molarity of the amount of N1-PA present within the co-assembly, rather than the total number of PA molecules. For DCC-receptor blocking experiments, anti-DCC receptor antibody (Santa Cruz Biotechnology, sc-515834) was applied at a concentration of 1:1000 concurrently with PA treatments.

Cell Viability. Prior to imaging, neurons were washed with 1×HBSS (Gibco, cat. no. 14-170-112) and then stained with a solution of calcein (1.43 μg/mL) and propidium iodide (2 μg/mL) prepared in 1×HBSS for 20-30 min at 37° C. Following staining, cells were washed twice with 1×HBSS. Imaging was performed using an Essen Bioscience IncuCyte S3 instrument located in the ANTEC facility at Northwestern University. In addition, media aliquots obtained after 24 h, 72 h, and 1 week of culture were analyzed using a CyQuant LDH Cytotoxicity Assay kit (Invitrogen, cat. no. C20301). Absorption measurements were performed with a BioTek Cytation3 Plate Reader and Imager located at the ANTEC facility at Northwestern University.

Scanning Electron Microscopy. Neurons were treated with PA in solution for 24 h. Samples were fixed in 2.5% glutaraldehyde (Electron Microscopy Sciences, Fisher Scientific, cat. no. 50-262-18)/2% paraformaldehyde (Electron Microscopy Sciences, Fisher Scientific, cat. no. 043368.9M) solution prepared in 1×PBS for at least 20 min and were then subjected to an ethanol exchange in preparation for critical point drying (CPD). Every 10 min, the samples were placed in a higher percentage ethanol solution in the following order: 30%, 40%, 50%, 60%, 70%, 80%, 85%, 90%, 95%, and 100%. Samples were left in 100% ethanol for at least 30 min and then until they could be critical point dried. CPD was performed on a Tousimis Samdri-795. Samples were purged for 20 min. Dried samples were preserved under a vacuum until the day of SEM imaging. Immediately prior to imaging, samples were coated with osmium using an SPF osmium coater located at the NUANCE facility at Northwestern University. Samples were coated twice at a thickness of 8 nm, resulting in a final osmium coating thickness of 16 nm. SEM imaging was performed on a Hitachi SU8030 instrument located in the NUANCE facility at Northwestern University, operating at an accelerating voltage of 2.0 kV.

Microfluidics Device Assay. XonaChip microfluidic devices (Xona Microfluidics, no. XC450) were obtained from Xona Microfluidics. Prior to neuron seeding, devices were prepared according to the manufacturer's recommendations.⁸⁸ In summary, first, devices were coated in XonaChip Pre-Coat solution and then washed twice with 1×PBS (Gibco, cat. no. 10010). Next, 0.01 mg/mL poly-D-lysine hydrobromide (MW 70,000-150,000; Sigma-Aldrich, cat. no. P6407) prepared in Milli-Q water was added to each well, and samples were incubated overnight. The following day, wells were washed twice with 1×PBS. A neural medium was then added to wells and then removed immediately prior to neuron seeding. Neurons were carefully added into the upper left well at a density of 90,000 cells/20 μL. After 5 min, media were added to all wells. The day following the dissection, all media were removed. Untreated media were added to the left side of the device (soma compartments), and treated media were added to the right side of the device in a higher volume to promote diffusion toward the soma compartments. Additional media were added to the device every 1-2 days to account for media loss caused by evaporation. Neurons were left in vitro for 1 week prior to fixation. Neurons were fixed in 4% paraformaldehyde and stained following a modified version of the manufacturer's protocol.⁸⁸

Immunocytochemistry. Neurons were fixed in 4% paraformaldehyde (Fisher Scientific, cat. no. 043368.9M) for 10 min, after which they were washed with 1×PBS (Gibco, cat. no. 10010), blocking buffer prepared with 5% NHS (Gibco, cat. no. 16-050-122) and 0.1% Triton X-100 (Fisher Bioreagents, cat. no. BP151-500) in 1×PBS. Fresh blocking buffer was added for a 2.5-3 h incubation. After this, the blocking buffer was replaced with the desired primary antibody solution (prepared in blocking buffer), which was incubated at 4° C. overnight. The next day, samples were washed with blocking buffer three times for 15 min each. 350 μL of the secondary antibody solution was added. Samples were incubated with a secondary antibody solution prepared in blocking buffer for a minimum of 2 h, protected from light. Following one wash in blocking buffer, the nuclear stain DAPI (1:1000, Thermofisher, D1306) was applied for 10 min. Samples were then washed three times with blocking buffer for 20 min each, followed by three washes with 1×PBS for 20 min each. Coverslips were immediately mounted onto glass slides with Immuno-Mount solution (ThermoScientific, cat. no. 9990402) and stored at 4° C. until imaging. Primary antibodies: 1:2000 MAP2 (Rb, Biolegend, 840601), 1:1000 SMI312 (Ms, Biolegend, NC1239357), 1:2000 Tuj1 (Rb, Biolegend, 802001), 1:1000 Tuj1 (Chk, Abcam, ab41489), 1:1000 anti-FOX3 (NueN) (Ms, Biolegend, 834501), 1:500 Syp (Rb, Abeam, ab32127), and 1:500 PSD95 (Ms, NIH NeuroMab/Antibodies Inc., 75-028). Secondary antibodies: 1:1000 Alexa Fluor 488 (Ms, Invitrogen, A-21202), 1:1000 Alexa Fluor 488 (Rb, Invitrogen, A11008), 1:1000 Alexa Fluor 555 (Rb, Invitrogen, A-31572), 1:1000 Alexa Fluor 647 (Ms, Invitrogen, A31571), 1:1000 Alexa Fluor 647 (Chk, Invitrogen, A-21449), 1:1000 Alexa Fluor 546 (Chk, Invitrogen, A11040), and 1:1000 Alexa Fluor 555 (Chk, Invitrogen, A21437).

Imaging and Image Analysis. All confocal images were taken on a Nikon A1R (B) GaAsP instrument at the Center for Advanced Microscopy/Nikon Imaging Center (CAM) at Northwestern University. Image analysis was performed with either ImageJ's NeuronJ software for axon length measurements or Imaris software for the network area. For microfluidic device neurite analysis, individual images were taken of the treatment compartment of each device, encapsulating the entire surface area. Quantification of each image was performed with ImageJ software. The integrated density of fluorescent pixels was measured in 20 μm-wide boxes encapsulating the entire height of the image across the length of the treatment chamber up to 800 μm. After this, the integrated density of fluorescent pixels was measured in 20 μm wide boxes every 100 μm. The integrated density of fluorescent pixels was then normalized to the highest obtained value in each image.

Western Blot. Neurons were treated in solution. At the desired time point, cells were mechanically disrupted using a cell scraper, and protein was extracted in Pierce RIPA buffer (ThermoScientific, cat. no. 89901) with a Halt protease and phosphatase inhibitor cocktail (ThermoScientific, cat. no. 1861281). Extracted protein was horn sonicated prior to quantification using a Pierce BCA Protein Assay Kit (Thermo Scientific, cat. no. 23225). Samples were run through 10 or 15 well Mini-PROTEAN TGX Stain-Free 4-20% agarose gels (Bio Rad, cat. no. 4568094, cat. no. 4568096) at 120 V. Gels were then transferred to nitrocellulose membranes (Bio Rad, cat. no. 1620094) at 100 V for 1 h. Ponceau solution was used to confirm the protein transfer to membranes. Prior to staining, membranes were blocked with nonfat milk (Bio Rad, cat. no. 1706404) prepared in 1×TBST (Fisher Bioreagents, cat. no. BP2471-500) for 30 min and then washed with a 1××TBST solution. Primary antibodies were applied, and samples were incubated for 30 min at room temperature and overnight at 4° C. The next day, the primary antibody was removed, and membranes were washed three times with 1×TBST. The secondary antibody was then applied in milk for 1 h. Membranes were then washed three times with 1×TBST buffer prior to imaging. Immediately prior to imaging, Radiance Q (Azure Biosystems, cat. no. AC2101) or Radiance Plus (Azure Biosystems, cat. no. AC2103) was applied to the membranes. An Azure300 instrument located in ANTEC at Northwestern University was used to develop Western blot images. ImageJ software was used to perform densitometry analysis, and signals were normalized to either total receptor, when relevant, or actin. All Western blots were performed in triplicate from at least two separate dissections. Primary antibodies: 1:1000 anti-PI3K (Rb, Cell Signaling, 4292), 1:500 anti-GAP43 (Rb, Cell Signaling, 5307S), 1:1000 anti-pPLCγ (Rb, Cell Signaling, 2821), 1:1000 anti-pERK1/2 (Cell Signaling, cat. no. 8544), 1:1000 anti-ERK1/2 (Cell Signaling, cat. no. 9102), 1:500 anti-Rac1/2 (Ms, Abcam, ab155938), 1:1000 anti-MAP2 (Rb, Biolegend, cat. no. 840601), 1:1000 anti-SMI312 (Ms, Biolegend, NC1239357), 1:4000 anti-Tuj1 (Rb, Biolegend, 802001), 1:2000 anti-NueN (Ms, Biolegend, 834501), 1:1000 anti-Syp (Rb, Abcam, ab32127), 1:20000 anti-PSD95 (Ms, NIH NeuroMab/Antibodies Inc., 75-028), and 1:2000 anti-actin (Rb, Sigma-Aldrich, A2066). Secondary antibodies: Goat antirabbit IgG (H+L), HRP (Thermofisher, 31460), Goat antimouse IgG (H+L), and HRP (Thermofisher, 31430).

Electrophysiology. A 48-well microelectrode array (MEA) plate with 16 PEDOT electrodes per well was coated with PEI and laminin according to Axion Biosystems protocols.⁸⁹ E16 primary mouse cortical neurons were seeded at a density of 50,000 cells/well and cultured for 21 days. Every 4-5 days, half of the medium was removed from each well and replaced with fresh media. On days 1, 6, 12, and 19, cells were treated with recombinant mouse netrin-1 (Fisher Scientific, cat. no. 1109N1025) or cyclic peptide. On days 1, 12, and 19, cells were treated with E₂-PA, N1-PA, or L-N1-PA. Spontaneous network and synchronized activity were recorded using Axion Biosystems Maestro 768 channel amplifier and Axion Integrated Studios (AxIS) v2.4 software. The amplifier recorded from all channels simultaneously using a gain of 1200× and a sampling rate of 12.5 kHz/channel. After passing the signal through a Butterworth band-pass filter (300-5000 Hz), on-line spike detection (threshold of 6× the root-mean-square of noise on each channel) was done with the AxIS adaptive spike detector. Recordings were performed on day 21. All recordings were conducted at 37° C. with 5% CO₂/95% O₂. Spontaneous network activity was recorded for 5 min. Active electrodes were defined as having >5 spikes/min, and only wells with over 10 active electrodes during the baseline-recording period were used in the analysis. Synchronized activity was defined as spike and burst activity that occurred on 25% of the electrodes or more in a well within 100 ms of each other. All data reflects well-wide averages.

Statistical Analysis. Data analysis was performed with GraphPad Prism (version 9.5.0). Comparisons among three or more groups were conducted using one-way ANOVA with either a Tukey's or Kruskal-Wallis multiple comparisons test. Comparisons between neurons treated either with or without the presence of anti-DCC antibody were conducted using two-way ANOVA with a Tukey's multiple comparisons test. The statistical tests and parameters used for each experiment are reported in the corresponding figure legends. For confocal data, error bars represent at least 30 images from two to three separate dissections. For Western blot experiments, error bars represent densitometry results calculated across three distinct blots from two to three separate dissections. For XonaChip experiments, error bars represent three separate devices. For MEA plate experiments, error bars represent eight distinct wells from one dissection. All error bars shown in the graphs represent the standard error mean unless otherwise indicated.

### In Vivo Studies

IACUC Standard Statement: All animal housing and procedures were performed in accordance with the Public Health Service Policy on Humane Care and Use of Laboratory Animals. All procedures were approved by the Northwestern University Institutional Animal Care and Use Committee.

PA Treatments: PA was sterilely prepared at a concentration of 10 mM in 0.9% w/v saline as previously described. To maintain sterility, all sonication steps were performed using bath sonication for 30 minutes.

Severe Spinal Cord Contusion Model: A total of 25 8-10 week old female CD1 mice (Charles River) were used for this study (Sham: N=16, E₂-PA: N=16, OC-E₂-PA: N=5, N1-PA: N=5, OC-N1-PA: N=5). All surgical tools were autoclaved and sterilized. Animals were anesthetized with 2.5% isoflurane in 100% O₂ (VetEquip) and maintained at 2.5% isoflurane in 100% O₂ for the duration of the procedure. In preparation for surgery, anesthetized mice were shaved and sterilized with washes of betadine and 70% ethanol at the surgical site. Baytril was administered subcutaneously on the left-hand side of the animal. A laminectomy was then performed at the T10-T12 site on the spinal cord. In preparation for the contusion, the spinal cord was immobilized with stabilizing forceps. An IH-0400 Impactor (Precision Systems and Instrumentation, LLC) equipped with a 1.25 mm impactor tip was used to deliver a severe contusion. Contusion parameters were set as follows: force=85 K dynes, dwell time=60 sec. Following the contusion, the wound was closed with AUTOCLIP 9 mm wound clips (BD Biosciences). Buprenex was administered subcutaneously along with Meloxicam on the right-hand side of the animal. Animals recovered on an electrical heating pad while being closely monitored. Mice were then moved to cages filled with Alpha-dry bedding with food placed on the floor. 24 hours after injury, mice were evaluated for hind limb movement. Animals with BMS (Basso mouse scale for locomotion)^{[21]} scores higher than 0 were removed from the study. Mice receiving treatment were anesthetized with 2.5% isoflurane in 100% O₂ and maintained at 2.5% isoflurane in 100% O₂ for the duration of the procedure. The surgical site was sterilized with betadine and wound clips were removed. The mouse was immobilized on a stereotactic stage. A Hamilton syringe equipped with a female Leur adaptor (World Precision Instruments) was loaded with a pulled glass capillary micropipette tip (Sutter Instruments) with an outer diameter of 100 μm. Treatments were injected just dorsal to the contusion site in the middle of the spinal cord at a depth of 750 μm. 4-6 μL of treatment was delivered. A stereotaxic Kopf apparatus was used to position the micropipette tip. Following treatment, the wound was again closed with wound clips. Buprenex was administered subcutaneously along with Meloxicam on the right-hand side of the animal. Animals recovered on an electrical heating pad while being closely monitored. Mice were then moved to cages filled with Alpha-dry bedding with food placed on the floor for the duration of the study. Following the surgeries, animals were closely monitored for signs of distress and infection—Baytril, Meloxicam, and Buprenex were administered following veterinary recommendations. The bladders of the mice were manually expressed daily for the duration of the study. BMS scoring was performed weekly at the same time and in the same location for consistency.

BMS (Basso Mouse Scale for Locomotion) Scoring: Two BMS scorers were double blinded for the duration of the study. Once a week at the same time and place, scorers would evaluate each mouse for hind limb movement and rate the mouse using BMS (Basso mouse scale for locomotion)^{[21]} scoring. Mice were evaluated while ambulating within a cork-bottomed container for approximately 3-5 minutes.

Animal Euthanasia and Tissue Processing: At the end of the 3-month study, animals were transcardially perfused with 4% PFA (Sigma-Aldrich) prepared in a 7.4 pH isotonic solution of 0. μM phosphate buffer. After removing the fixed spinal cords from the animal, the spinal cords were placed in fresh 4% PFA solution for 6 hours and then transferred to a 30% sucrose solution prepared in 1×PBS overnight at 4° C. Once the spinal cords were completely transfused with sucrose solution, indicated by the spinal cords sinking to the bottom of the sucrose solution they were stored in, they were frozen in a solution of 30% sucrose (Sigma-Aldrich) and 15% gelatin (Sigma-Aldrich) prepared in 1×PBS. 40 m thick tissue slices were sectioned on a Leica CM1850 cryostat in preparation for immunostaining.

Immunostaining: Free-floating spinal cord tissue slices were washed once with and then incubated in a 1% NHS (Gibco, Cat. No. 16-050-122), 0.02% Triton-X (Fisher Bioreagents, Cat. No. BP151-500) blocking buffer solution prepared in 1×PBS (Gibco, Cat. No. 10010) for 2.5-3 hours. The primary antibody solution, prepared in blocking buffer, was then applied to tissue samples overnight at 4° C. The next day, samples were washed three times with blocking buffer solution before being incubated with the secondary antibody solution, prepared in blocking buffer, for 2 hours at room temperature. Next, to stain nuclei, DAPI (1:500, Thermofisher, D1306) was applied for 10 minutes. Samples were then washed three times with blocking buffer solution and three times with 1×PBS for 15 minutes each. Samples were immediately mounted onto glass coverslips with Immuno-Mount solution (ThermoScientific, Cat. No. 9990402) followed by imaging.

Primary antibodies: 1:1000 GFAP (Rb, Dako, Z0334), 1:1000 anti-laminin (Rb, Sigma-Aldrich, L9393), 1:1000 anti-5HT-H209 (Mouse, Novus Biologicals, NB120-16007), 1:100 anti-CD31 (Rb, BD Pharmigen, 550274), 1:2000 anti-Neurofilament (Ms, Millipore, MAB1592), 1:2000 anti-GAP43 (Rb, Cell Signaling, 8945), 1:1000 anti-Nestin (Rb, Millipore, ABD69), anti-NueN (Ms, Abeam, ab104224), anti-Tau (Chk, Abeam, ab75714).

Secondary antibodies: 1:1000 Alexa Fluor 488 (Ms, Invitrogen, A-21202), 1:1000 Alexa Fluor 488 (Rb, Invitrogen, A1008), 1:1000 Alexa Fluor 555 (Rb, Invitrogen, A-31572), 1:1000 Alexa Fluor 647 (Ms, Invitrogen, A31571), 1:1000 Alexa Fluor 647 (Chk, Invitrogen, A-21449), 1:1000 Alexa Fluor 546 (Chk, Invitrogen, A11040), 1:1000 Alexa Fluor 555 (Chk, Invitrogen, A21437).

Dot Blot: Protein was extracted from spinal cord tissue samples with Pierce RIPA buffer (ThermoScientific, Cat. No. 89901) supplemented with Halt Protease and Phosphatase Inhibitors (ThermoScientific, Cat. No. 1861281) and then horn sonicated (Branson). The protein content of each sample was assessed using a Pierce™ BCA Protein Assay Kit (Thermo Scientific, Cat. No. 23225). Electrophoresis was performed to separate proteins through an agarose gel. Protein was then transferred to nitrocellulose membranes (Bio Rad, Cat. No. 1620094) at 100V for 1 hour. Prior to application of the first primary antibody, membranes were incubated in a 5% nonfat milk (Bio Rad, Cat. No. 1706404) solution prepared in 1×TBST (Fisher Bioreagents, Cat. No. BP2471-500). The primary antibody was then applied overnight, incubating at 4° C. The next day, membranes were washed in a series of 1×TBS and 1×TBST solutions prior to incubating with secondary antibody solution for 1 hour at room temperature. Samples were then washed again in a series of 1×TBS and 1×TBST solutions. Immediately prior to imaging, membranes were incubated in Radiance Q (Azure Biosystems, Cat. No. AC2101) or Radiance Plus (Azure Biosystems, Cat. No. AC2103) solutions. Imaging was performed with an Azure300. Densitometry analysis was performed with Fiji software and normalized to either total receptor, when relevant, or actin.

Statistical Analysis for In Vivo Studies. Data analysis was performed with GraphPad Prism software (Version 9.5.1). Comparisons among three or more groups were conducted using one-way ANOVA with a Tukey's multiple comparisons test unless otherwise indicated. The statistical tests and parameters used for each experiment are reported in the corresponding figure legends. All error bars shown in graphs represent the standard error mean unless otherwise indicated.

1. A netrin-1 mimetic peptide amphiphile comprising a hydrophobic tail, a structural peptide segment, a charged peptide segment, and a netrin-1 mimetic sequence.

2. The netrin-1 mimetic peptide amphiphile of claim 1, wherein:
a) the hydrophobic tail comprises an 8-24 carbon alkyl chain (C8-24);
b) the structural peptide segment has a propensity for forming j-sheet conformations;
c) the charged peptide segment comprises an acidic, basic, or zwitterionic peptide segment; and/or
d) the netrin-1 mimetic sequence is attached to the charged peptide segment by a linker.

3. The netrin-1 mimetic peptide amphiphile of claim 1, wherein the structural peptide segment comprises 2 to 8 non-polar residues, and/or the charged peptide segment comprises EE, EEE, or EEEE (SEQ ID NO: 4) or KK, KKK, or KKKK (SEQ ID NO: 12).

4. The netrin-1 mimetic peptide amphiphile of claim 3, wherein the structural peptide segment comprises V₂A₂(SEQ ID NO: 2) or A₂V₂(SEQ ID NO: 5).

5. The netrin-1 mimetic peptide amphiphile of claim 1, wherein the netrin-1 mimetic sequence comprises IDP.

6. The netrin-1 mimetic peptide amphiphile of claim 5, wherein the netrin-1 mimetic peptide sequence comprises CIDPC (SEQ ID NO: 1).

7. The netrin-1 mimetic peptide amphiphile of claim 6, wherein the sequence CIDPC (SEQ ID NO: 1) is cyclized via disulfide bonding between the terminal cysteine residues.

8. The netrin-1 mimetic peptide amphiphile of claim 1, wherein the linker comprises GG.

9. The netrin-1 mimetic peptide amphiphile of claim 1, wherein the peptide amphiphile comprises C₈₋₂₄-V₂A₂E₂G₂CIDPC (SEQ ID NO: 11).

10. A nanofiber comprising the netrin-1 mimetic peptide amphiphile of claim 1.

11. The nanofiber of claim 10, further comprising one or more filler peptide amphiphiles, wherein the filler peptide amphiphiles comprise a hydrophobic tail, a structural peptide segment, and a charged peptide segment, and wherein the filler peptide amphiphiles do not comprise the netrin-1 mimetic sequence.

12. The nanofiber of claim 11, wherein the filler peptide amphiphiles do not comprise a bioactive segment.

13. The nanofiber of claim 11, comprising 10-40% netrin-1 mimetic peptide amphiphiles and 60-90% filler peptide amphiphiles

14. The nanofiber of claim 11, comprising about 30% netrin-1 mimetic peptide amphiphiles and about 70% filler peptide amphiphiles.

15. A pharmaceutical composition comprising the peptide amphiphile of claim 1.

16. A method of treating a nervous system injury or disease in a subject in need thereof comprising administering a therapeutically effective amount of the pharmaceutical composition of claim 15 to the subject.

17. A scaffold comprising the nanofiber of claim 10.

18. A cell or an organoid cultured on the scaffold of claim 17.

19. A method of culturing cells or an organoid, comprising contacting the cells or the organoid with the scaffold of claim 17.

20. A system comprising the scaffold of claim 17 and a cell or an organoid cultured on the scaffold.


SEQUENCE LISTING (18 sequences)

SEQ ID NO: 1 - protein, 5 aa, synthetic construct
CIDPC

SEQ ID NO: 2 - protein, 4 aa, synthetic construct
VVAA

SEQ ID NO: 3 - protein, 6 aa, synthetic construct
VVVAAA

SEQ ID NO: 4 - protein, 4 aa, synthetic construct
EEEE

SEQ ID NO: 5 - protein, 4 aa, synthetic construct
AAVV

SEQ ID NO: 6 - protein, 6 aa, synthetic construct
AAAVVV

SEQ ID NO: 7 - protein, 5 aa, synthetic construct
VVAAA

SEQ ID NO: 8 - protein, 5 aa, synthetic construct
VVVAA

SEQ ID NO: 9 - protein, 5 aa, synthetic construct
SIDPS

SEQ ID NO: 10 - protein, 6 aa, synthetic construct
VVAAEE

SEQ ID NO: 11 - protein, 13 aa, synthetic construct
VVAAEEGGCIDPC

SEQ ID NO: 12 - protein, 4 aa, synthetic construct
KKKK

SEQ ID NO: 13 - protein, 4 aa, synthetic construct
GGGG

SEQ ID NO: 14 - protein, 5 aa, synthetic construct
EIDPK

SEQ ID NO: 15 - protein, 5 aa, synthetic construct
DIDPK

SEQ ID NO: 16 - protein, 4 aa, synthetic construct
AAGG

SEQ ID NO: 17 - protein, 6 aa, synthetic construct
AAAGGG

SEQ ID NO: 18 - protein, 5 aa, synthetic construct
VEVAA